(19) United States
(12) Patent Application Publication
(10) Pub. No.: US 2012/0195858 A1
(43) Pub. Date: Aug. 2, 2012

(54) RESPONSIVENESS TO ANGIOGENESIS INHIBITORS

(76) Inventors: Dorothee Foernzler, Lenzburg (CH); Paul Delmar, Basel (CH); Stefan Scherer, Basel (CH); Diether Lambrechts, Kessel-lo (Leuven) (BE); Peter Carmeliet, Blanden (BE)

(21) Appl. No.: 13/388,840

(22) PCT Filed: Aug. 3, 2010
(86) PCT No.: PCT/EP10/04761

§ 371 Date: Apr. 19, 2012

(30) Foreign Application Priority Data
Aug. 4, 2009 (EP) 09167184.2

(51) Int. Cl. A61K 38/21; A61P 35/00; C07H 21/04; A61K 39/395; C12Q 1/68

(52) U.S. Cl. 424/85.7; 424/133.1; 435/6.11; 536/24.31; 536/24.33

(57) ABSTRACT

The present invention relates to methods for improving the overall survival of a patient suffering from a malignant disease or a disease involving physiological and pathological angiogenesis by treatment with an angiogenesis inhibitor, such as bevacizumab, by determining the presence of one or more variant alleles of the vascular endothelial growth factor receptor 1 (VEGFR-1) gene. The present invention further provides methods for improving the progression-free survival of a patient suffering from a malignant disease or a disease involving physiological and pathological angiogenesis by treatment with an angiogenesis inhibitor, such as bevacizumab, by determining the presence of one or more variant alleles of the VEGFR-1 gene. The present invention also provides for methods for assessing the responsiveness of a patient to an angiogenesis inhibitor by determining the presence of one or more variant alleles of the VEGFR-1 gene.

The present invention relates to a method for improving overall survival of a patient suffering from a malignant disease or a disease involving physiological and pathological angiogenesis by treatment with an angiogenesis inhibitor, such as bevacizumab, by determining the presence of one or more variant alleles of the vascular endothelial growth factor receptor 1 (VEGFR-1) gene. The present invention also relates to a method for improving progression-free survival of a patient suffering from a malignant disease or a disease involving physiological and pathological angiogenesis by treatment with an angiogenesis inhibitor, such as bevacizumab, by determining the presence of one or more variant alleles of the vascular endothelial growth factor receptor 1 (VEGFR-1) gene. The present invention further provides for methods for assessing the responsiveness of a patient to an angiogenesis inhibitor by determining the presence of one or more variant alleles of the vascular endothelial growth factor receptor 1 (VEGFR-1) gene.

Angiogenesis contributes to benign and malignant diseases such as cancer development and, especially in cancer, is necessary for primary tumor growth, invasiveness and metastasis. In order to grow, a tumor must undergo an angiogenic switch. Vascular endothelial growth factor (VEGF) is required to induce this angiogenic switch. VEGF and the genes in the VEGF pathway are considered important mediators of cancer progression. The VEGF gene family includes the VEGF gene, also referred to as VEGFA, homologues to VEGF including, placenta growth factor (PlGF), VEGFB, VEGFC, VEGFD, the VEGF receptors, including VEGFR-1 and VEGFR-2 (also referred to as FLT1 and FLK1/KDR, respectively), the VEGF inducers, including hypoxia-inducible factors HIF1α, HIF2 α, and the oxygen sensors PHD1, PHD2 and PHD3.

The importance of this pathway in cancer cell growth and metastasis has led to the development of anti-angiogenesis agents for use in cancer therapy. These therapies include, among others, bevacizumab, pegaptanib, sunitinib, sorafenib and vatalanib. Despite significantly prolonged survival obtained with angiogenesis inhibitors, such as bevacizumab, patients still succumb to cancer. Further, not all patients respond to angiogenesis inhibitor therapy. The mechanism underlying the non-responsiveness remains unknown. Moreover, angiogenesis inhibitor therapy is associated with side effects, such as gastrointestinal perforation, thrombosis, bleeding, hypertension and proteinuria.

Accordingly, there is a need for methods of determining which patients respond particular well to angiogenesis inhibitor therapy.

The present invention, therefore, provides a method for improving the overall survival of a patient suffering from a malignant disease or a disease involving physiological and pathological angiogenesis by treatment with an angiogenesis inhibitor, said method comprising the following steps:

(a) determining from a patient sample if the patient suffering from a malignant disease or a disease involving physiological and pathological angiogenesis has one or more variant alleles of the VEGFR-1 gene; and
(b) administering an angiogenesis inhibitor to the patient having one or more variant alleles of the VEGFR-1 gene.

The malignant disease or a disease involving physiological and pathological angiogenesis may be pancreatic cancer. The angiogenesis inhibitor may be bevacizumab.

Accordingly, the present invention relates to a method for improving the overall survival of a patient suffering from a malignant disease or a disease involving physiological and pathological angiogenesis by treatment with an angiogenesis inhibitor, said method comprising the following steps:

(a) obtaining a sample from a patient suffering from a malignant disease or a disease involving physiological and pathological angiogenesis;
(b) determining if the patient has one or more variant alleles of the VEGFR-1 gene; and
(c) administering an angiogenesis inhibitor to the patient having one or more variant alleles of the VEGFR-1 gene.

The malignant disease or a disease involving physiological and pathological angiogenesis may be pancreatic cancer. The angiogenesis inhibitor may be bevacizumab.

Therefore, the present invention relates to a method for improving the progression-free survival of a patient suffering from a malignant disease or a disease involving physiological and pathological angiogenesis by treatment with an angiogenesis inhibitor, said method comprising the following steps:

(a) determining from a patient sample if the patient suffering from a malignant disease or a disease involving physiological and pathological angiogenesis has one or more variant alleles of the VEGFR-1 gene; and
(b) administering an angiogenesis inhibitor to the patient having one or more variant alleles of the VEGFR-1 gene.

The malignant disease or a disease involving physiological and pathological angiogenesis may be pancreatic cancer or renal cell cancer. The angiogenesis inhibitor may be bevacizumab.

The present invention relates to a method for improving the progression-free survival of a patient suffering from a malignant disease or a disease involving physiological and pathological angiogenesis by treatment with an angiogenesis inhibitor, said method comprising the following steps:

(a) obtaining a sample from a patient suffering from a malignant disease or a disease involving physiological and pathological angiogenesis;
(b) determining if the patient has one or more variant alleles of the VEGFR-1 gene; and
(c) administering an angiogenesis inhibitor to the patient having one or more variant alleles of the VEGFR-1 gene.

The malignant disease or a disease involving physiological and pathological angiogenesis may be pancreatic cancer or renal cell cancer. The angiogenesis inhibitor may be bevacizumab.

The present invention provides an in vitro method for the identification of a responder for or a patient sensitive to an angiogenesis inhibitor, said method comprising determining from a patient sample if the patient suffering from a malignant disease or a disease involving physiological and pathological angiogenesis has one or more variant alleles of the VEGFR-1 gene whereby the presence of the one or more variant alleles of the VEGFR-1 gene is indicative for a responding patient or is indicative of a sensitivity of the patient to the angiogenesis inhibitor. The malignant disease or a disease involving physiological and pathological angiogenesis may be pancreatic cancer or renal cell cancer. The angiogenesis inhibitor may be bevacizumab.

Accordingly, the present invention relates to an in vitro method for the identification of a responder for or a patient sensitive to an angiogenesis inhibitor, said method comprising the following steps:

(a) obtaining a sample from a patient suffering from a malignant disease or a disease involving physiological and pathological angiogenesis; and
(b) determining if the patient has one or more variant alleles of the VEGFR-1 gene; whereby the presence of the one or more variant alleles of the VEGFR-1 gene is indicative for a responding patient or is indicative for a sensitivity for the patient to the angiogenesis inhibitor. The malignant disease or a disease involving physiological and pathological angiogenesis may be pancreatic cancer or renal cell cancer. The angiogenesis inhibitor may be bevacizumab.

In accordance with the embodiments herein disclosed, the present invention provides the means and methods of identification of a patient or group of patients suffering from a malignant disease or a disease involving physiological and pathological angiogenesis who benefit from treatment with angiogenesis inhibitors, in particular to treatment with bevacizumab.

In the present invention, variations in the VEGFR-1 gene were surprisingly identified as markers/predictors for overall survival and/or progression-free survival to treatment with an angiogenesis inhibitor. The terms “marker” and “predictor” can be used interchangeably and refer to specific allele variants of genes, in particular the VEGFR-1 gene (see, for example, Ensembl ID ENSG00000102755). The variation or marker may also be referred to as a single nucleotide polymorphism (SNP).

Preferably the one or more variant alleles are in the region encompassing the tyrosine kinase domain of the VEGFR-1 gene. In particular, preferably the one or more variant alleles are in the region encoding the tyrosine kinase domain of the VEGFR-1 gene. More preferable the one or more variant alleles are in the region encompassing exons 25 to 30 of the VEGFR-1 gene. Even more preferably, the one or more variant alleles are in an intron of the VEGFR-1 gene. Even more preferably the one or more variant alleles of the VEGFR-1 gene are selected from the group consisting of rs9582036 (SEQ ID NO. 2), rs9554316 (SEQ ID NO. 1), rs9554320 (SEQ ID NO. 4) and/or rs9513070 (SEQ ID NO. 3). Most preferably, the one or more variant alleles of the VEGFR-1 gene are selected from the group consisting of rs9582036 (SEQ ID NO. 2) and/or rs9554316 (SEQ ID NO. 1) or the group consisting of rs9554316 (SEQ ID NO. 1) and/or rs9513070 (SEQ ID NO. 3).

Accordingly, the invention provides a method for improving overall survival of a patient suffering from a metastatic pancreatic cancer by treatment with an angiogenesis inhibitor comprising the following steps:

(a) determining from a patient sample if the patient suffering metastatic pancreatic cancer has one or more variant alleles of the VEGFR-1 gene, wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(b) administering the angiogenesis inhibitor to the patient having one or more variant alleles of the VEGFR-1 gene.

The angiogenesis inhibitor may be bevacizumab.

The invention relates to a method for improving overall survival of a patient suffering from a metastatic pancreatic cancer by treatment with an angiogenesis inhibitor comprising the following steps:

(a) obtaining a sample from a patient suffering from metastatic pancreatic cancer;
(b) determining from a patient sample if the patient has one or more variant alleles of the VEGFR-1 gene wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(c) administering the angiogenesis inhibitor to the patient having one or more variant alleles of the VEGFR-1 gene.

The angiogenesis inhibitor may be bevacizumab.

The invention relates to a method for improving progression-free survival of a patient suffering from a metastatic pancreatic cancer by treatment with an angiogenesis inhibitor comprising the following steps:

(a) determining from a patient sample if the patient suffering metastatic pancreatic cancer has one or more variant alleles of the VEGFR-1 gene wherein the one or more variant alleles are selected from the group consisting of rs9554316 (SEQ ID NO. 1), SNPs rs9582036 (SEQ ID NO. 2), rs9513070 (SEQ ID NO. 3) and rs9554320 (SEQ ID NO.4); and
(b) administering the angiogenesis inhibitor to the patient having one or more variant alleles of the VEGFR-1 gene.

The angiogenesis inhibitor may be bevacizumab.

The invention relates to a method for improving progression-free survival of a patient suffering from a metastatic pancreatic cancer by treatment with an angiogenesis inhibitor comprising the following steps:

(a) obtaining a sample from a patient suffering from metastatic pancreatic cancer;
(b) determining from a patient sample if the patient has one or more variant alleles of the VEGFR-1 gene wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(c) administering the angiogenesis inhibitor to the patient having one or more variant alleles of the VEGFR-1 gene.

The angiogenesis inhibitor may be bevacizumab.

Accordingly, the invention provides a method for improving progression-free survival of a patient suffering from a renal cell cancer by treatment with an angiogenesis inhibitor comprising the following steps:

(a) determining from a patient sample if the patient renal cell cancer has one or more variant alleles of the VEGFR-1 gene wherein the one or more variant alleles are selected from the group consisting of rs9554316 (SEQ ID NO. 1) and rs9513070 (SEQ ID NO. 3); and
(b) administering the angiogenesis inhibitor to the patient having one or more variant alleles of the VEGFR-1 gene.

The angiogenesis inhibitor may be bevacizumab.

The invention relates a method for improving progression-free survival of a patient suffering from a renal cell cancer by treatment with an angiogenesis inhibitor comprising the following steps:

(a) obtaining a sample from a patient suffering from renal cell cancer;
(b) determining from a patient sample if the patient has one or more variant alleles of the VEGFR-1 gene wherein the one or more variant alleles are selected from the group consisting of rs9554316 (SEQ ID NO. 1) and rs9513070 (SEQ ID NO. 3); and
(c) administering the angiogenesis inhibitor to the patient having one or more variant alleles of the VEGFR-1 gene.

The angiogenesis inhibitor may be bevacizumab.

Besides the SNPs herein described, also with the scope of this invention are SNPs that are linked to the SNPs herein described. Linkage disequilibrium is defined in the context of the present invention as having a D′>0.8. To determine these estimates of disequilibrium (Dᵢⱼ) between pair-wise combinations of alleles the following definition was used: Dᵢⱼ=hᵢⱼ−pᵢqⱼ whereby hᵢⱼ represents the frequency of the observed haplotype with allele i at locus 1 and allele j at locus 2, and pᵢ and qⱼ the frequencies of both alleles. The linkage disequilibrium coefficient D′ᵢⱼ is standardized by its maximum value as D′=D/Dₘₐₓ. If D<0, then Dₘₐₓ=−pq; if D>0, then Dₘₐₓ=p (1−q). The linked SNPs identified according to this definition may be located within the region encompassing exons 25 to 30 of the VEGFR-1 gene but also upstream or downstream from the region encompassing exons 25 to 30 of the VEGFR-1 gene. For example, using this method of defining linkage disequilibrium, linked SNPs include rs17619037 (SEQ ID NO. 5), rs9579177 (SEQ ID NO. 6), rs9579176 (SEQ ID NO. 7), rs9554319 (SEQ ID NO. 8), rs7996030 (SEQ ID NO. 9), rs9513075 (SEQ ID NO. 10), rs7993418 (SEQ ID NO. 11), rs9513071 (SEQ ID NO. 12), rs11620238 (SEQ ID NO. 13), rs17618631 (SEQ ID NO. 14), rs12429309 (SEQ ID NO. 15), rs7982251 (SEQ ID NO 16), rs9508016 (SEQ ID NO. 17), rs7982957 (SEQ ID NO. 18) and rs3794400 (SEQ ID NO. 19).

The person skilled in the art will understand that other well-accepted and/or related methods of determining linkage equilibrium may be applied as well to determine additional SNPs linked to the SNPs herein disclosed, e.g., SNPs rs9554316 (SEQ ID NO. 1), rs9582036 (SEQ ID NO. 2), rs9513070 (SEQ ID NO. 3) and/or rs9554320 (SEQ ID NO.4). In the context of the present invention, for example, the correlation coefficient r² was used as an additional measure. To determine estimates of correlation between pair-wise combinations of alleles the following definition can be used: D=h−p₁q₁ whereby h represents the frequency of the observed haplotype with allele 1 at locus 1 and allele 1 at locus 2, and p₁ and q₁ are the frequencies of both alleles. When p₂ and q₂ are respectively defined as (1-p₁) and (1-q₁), the correlation coefficient between pairs of loci can be defined as

$$r = \frac{D}{\sqrt{{p}_{1} {p}_{2} {q}_{1} {q}_{2}}}$$

Accordingly, using this definition, linkage disequilibrium may be defined as having an r² equal or larger that 0.12 to rs9554316 (SEQ ID NO. 1), rs9582036 (SEQ ID NO. 2), rs9513070 (SEQ ID NO. 3) or rs9554320 (SEQ ID NO.4), for example. The threshold of r² equal or larger than 0.12 was applied because this r² value represents the lowest level of linkage disequilibrium between the four SNPs (SEQ ID NOs. 1 to 4) described within the scope of this invention and identified as markers/predictors for overall survival and/or progression-free survival to treatment with an angiogenesis inhibitor. Accordingly, using this alternative method of defining linkage disequilibrium, linked SNPs include, in addition to those identified above (i.e, SEQ ID NOs. 5 to 19), rs45455097 (SEQ ID NO. 40), rs1886233 (SEQ ID NO. 41), rs9554309 (SEQ ID NO. 42), rs11619230 (SEQ ID NO. 43), rs9554311 (SEQ ID NO. 44), rs4771233 (SEQ ID NO. 45), rs6491274 (SEQ ID NO. 46), rs7982639 (SEQ ID NO. 47), rs12877718 (SEQ ID NO. 48), rs10507382 (SEQ ID NO. 49), rs57354941 (SEQ ID NO. 50), rs17086497 (SEQ ID NO. 51), rs3794395 (SEQ ID NO. 52), rs9554317 (SEQ ID NO. 53), rs9513073 (SEQ ID NO. 54), rs9513074 (SEQ ID NO. 55), rs9508015 (SEQ ID NO. 56), rs2011950 (SEQ ID NO. 57), rs9513110 (SEQ ID NO. 58), rs9513112 (SEQ ID NO. 59), rs9513113 (SEQ ID NO. 60), rs9551471 (SEQ ID NO. 61), rs2296285 (SEQ ID NO. 62), rs9513116 (SEQ ID NO. 63), rs9551473 (SEQ ID NO. 64), rs7330109 (SEQ ID NO. 65), rs9508037 (SEQ ID NO. 66), rs1924981 (SEQ ID NO. 67), rs34140996 (SEQ ID NO. 68), rs7985584 (SEQ ID NO. 69), rs7992940 (SEQ ID NO. 70) and rs718273 (SEQ ID NO. 71).

In the context of the herein described invention, the linked SNPs herein described may be used alone, in combination with SNPs rs9554316 (SEQ ID NO. 1), rs9582036 (SEQ ID NO. 2), rs9513070 (SEQ ID NO. 3) and/or rs9554320 (SEQ ID NO.4) or in combination with other linked SNPs herein described in the methods and or uses herein described.

Accordingly, the present invention relates to variations in the VEGFR-1 gene including nucleotide substitution(s), deletion(s) or addition(s) at the position corresponding to the SNP. For example, for rs9582036 (SEQ ID NO. 2), rs9554316 (SEQ ID NO. 1), rs9554320 (SEQ ID NO. 4) and/or rs9513070 (SEQ ID NO. 3), the variations include a substitution(s), deletion(s) or addition(s) at position 51 of the rs9554316 (SEQ ID NO. 1), rs9582036 (SEQ ID NO. 2), rs9513070 (SEQ ID NO. 3) and/or rs9554320 (SEQ ID NO. 4). Accordingly, in a further embodiment of the present invention the one or more variant alleles of the VEGFR-1 gene are selected from the group consisting of:

(a) a sequence corresponding to rs9554316 (SEQ ID NO. 1) or having at least 80%, at least 85%, or more preferably at least 90%, or even more preferably, at least 95%, at least 96%, at least 97%, at least 98%, or at least 99% homology to rs9554316 (SEQ ID NO. 1) having a substitution, deletion or addition of at least one nucleotide corresponding to position 51 of rs9554316 (SEQ ID NO. 1);
(b) a sequence corresponding to rs9582036 (SEQ ID NO. 2) or having at least 80%, at least 85%, or more preferably at least 90%, or even more preferably, at least 95%, at least 96%, at least 97%, at least 98%, or at least 99% homology to rs9582036 (SEQ ID NO. 2) having a substitution, deletion or addition of at least one nucleotide corresponding to position 51 of rs9582036 (SEQ ID NO. 2);
(c) a sequence corresponding to rs9513070 (SEQ ID NO. 3) or having at least 80%, at least 85%, or more preferably at least 90%, or even more preferably, at least 95%, at least 96%, at least 97%, at least 98%, or at least 99% homology to rs9513070 (SEQ ID NO. 3) having a substitution, deletion or addition of at least one nucleotide corresponding to position 51 of rs9513070 (SEQ ID NO. 3); and
(d) a sequence corresponding to rs9554320 (SEQ ID NO. 4) or having at least 80%, at least 85%, or more preferably at least 90%, or even more preferably, at least 95%, at least 96%, at least 97%, at least 98%, or at least 99% homology to rs9554320 (SEQ ID NO. 4) having a substitution, deletion or addition of at least one nucleotide corresponding to position 51 of rs9554320 (SEQ ID NO. 4).

In the context of the herein described invention, the above described homology, with regard to the identified SNPs, applies to each embodiment herein described wherein specific SNPs are recited.

Further, the variations can include a substitution(s), deletion(s) or addition(s) at any position from position 1 to 50 and 52 to 101 of the rs9554316 (SEQ ID NO. 1), rs9582036 (SEQ ID NO. 2), rs9513070 (SEQ ID NO. 3) and/or rs9554320 (SEQ ID NO. 4). Accordingly, in a further embodiment of the present invention the one or more variant alleles of the VEGFR-1 gene are selected from the group consisting of:

(a) a sequence corresponding to rs9554316 (SEQ ID NO. 1) or having at least 80%, at least 85%, or more preferably at least 90%, or even more preferably, at least 95%, at least 96%, at least 97%, at least 98%, or at least 99% homology to rs9554316 (SEQ ID NO. 1), wherein position 51 is a T or G and having a substitution, deletion or addition of at least one nucleotide at any position from position 1 to 50 and 52 to 101;
(b) a sequence corresponding to rs9582036 (SEQ ID NO. 2) or having at least 80%, at least 85%, or more preferably at least 90%, or even more preferably, at least 95%, at least 96%, at least 97%, at least 98%, or at least 99% homology to rs9582036 (SEQ ID NO. 2) wherein position 51 is a C or A and having a substitution, deletion or addition of at least one nucleotide at any position from position 1 to 50 and 52 to 101;
(c) a sequence corresponding to rs9513070 (SEQ ID NO. 3) or having at least 80%, at least 85%, or more preferably at least 90%, or even more preferably, at least 95%, at least 96%, at least 97%, at least 98%, or at least 99% homology to rs9513070 (SEQ ID NO. 3) wherein position 51 is a G or A and having a substitution, deletion or addition of at least one nucleotide at any position from position 1 to 50 and 52 to 101; and
(d) a sequence corresponding to rs9554320 (SEQ ID NO. 4) or having at least 80%, at least 85%, or more preferably at least 90%, or even more preferably, at least 95%, at least 96%, at least 97%, at least 98%, or at least 99% homology to rs9554320 (SEQ ID NO. 4) wherein position 51 is a C or A and having a substitution, deletion or addition of at least one nucleotide at any position from position 1 to 50 and 52 to 101.

In the context of the herein described invention, the above described homology, with regard to the identified SNPs, applies to each embodiment herein described wherein specific SNPs are recited.

The present invention also relates to variations in the VEGFR-1 gene including nucleotide substitution(s), deletion(s) or addition(s) at the position corresponding to linked SNPs as well as other linked SNPs nearby but upstream or downstream from the VEGFR-1 gene, and located in the poly(A) specific ribonuclease subunit gene, Ensembl ID ENSG00000152520, (“PAN3 gene”) or in intergenic DNA regions. For example, variations in the VEGFR-1 gene include the linked SNPs rs17619037 (SEQ ID NO. 5), rs9579177 (SEQ ID NO. 6), rs9579176 (SEQ ID NO. 7), rs9554319 (SEQ ID NO. 8), rs7996030 (SEQ ID NO. 9), rs9513075 (SEQ ID NO. 10), rs7993418 (SEQ ID NO. 11), rs9513071 (SEQ ID NO. 12), rs12429309 (SEQ ID NO. 15), rs7982251 (SEQ ID NO 16), rs9508016 (SEQ ID NO. 17), rs7982957 (SEQ ID NO. 18), rs3794400 (SEQ ID NO. 19), rs3794395 (SEQ ID NO. 52), rs9554317 (SEQ ID NO. 53), rs9513073 (SEQ ID NO. 54), rs9513074 (SEQ ID NO. 55), rs9508015 (SEQ ID NO. 56), rs2011950 (SEQ ID NO. 57), rs9513110 (SEQ ID NO. 58), rs9513112 (SEQ ID NO. 59), rs9513113 (SEQ ID NO. 60), rs9551471 (SEQ ID NO. 61), rs2296285 (SEQ ID NO. 62), rs9513116 (SEQ ID NO. 63), rs9551473 (SEQ ID NO. 64), rs7330109 (SEQ ID NO. 65), rs9508037 (SEQ ID NO. 66), rs1924981 (SEQ ID NO. 67), rs34140996 (SEQ ID NO. 68), rs7985584 (SEQ ID NO. 69), rs7992940 (SEQ ID NO. 70) and rs718273 (SEQ ID NO. 71), and variations upstream of the VEGFR-1 gene, including the linked SNPs corresponding to variations in the PAN3 gene, such as rs45455097 (SEQ ID NO. 40), rs1886233 (SEQ ID NO. 41), rs9554309 (SEQ ID NO. 42), rs11619230 (SEQ ID NO. 43), and rs9554311 (SEQ ID NO. 44), and the linked SNPs corresponding to variations in the intergenic DNA regions, such as rs11620238 (SEQ ID NO. 13), rs17618631 (SEQ ID NO. 14), rs4771233 (SEQ ID NO. 45), rs6491274 (SEQ ID NO. 46), rs7982639 (SEQ ID NO. 47), rs12877718 (SEQ ID NO. 48), rs10507382 (SEQ ID NO. 49), rs57354941 (SEQ ID NO. 50), and rs17086497 (SEQ ID NO. 51). Accordingly, the variations include a substitution(s), deletion(s) or addition(s) at position 51 of rs17619037 (SEQ ID NO. 5), rs9579177 (SEQ ID NO. 6), rs9579176 (SEQ ID NO. 7), rs9554319 (SEQ ID NO. 8), rs7996030 (SEQ ID NO. 9), rs9513075 (SEQ ID NO. 10), rs7993418 (SEQ ID NO. 11), rs9513071 (SEQ ID NO. 12), rs11620238 (SEQ ID NO. 13), rs17618631 (SEQ ID NO. 14), rs12429309 (SEQ ID NO. 15), rs7982251 (SEQ ID NO 16), rs9508016 (SEQ ID NO. 17), rs7982957 (SEQ ID NO. 18), rs3794400 (SEQ ID NO. 19), rs45455097 (SEQ ID NO. 40), rs1886233 (SEQ ID NO. 41), rs9554309 (SEQ ID NO. 42), rs11619230 (SEQ ID NO. 43), rs9554311 (SEQ ID NO. 44), rs4771233 (SEQ ID NO. 45), rs6491274 (SEQ ID NO. 46), rs7982639 (SEQ ID NO. 47), rs12877718 (SEQ ID NO. 48), rs10507382 (SEQ ID NO. 49), rs57354941 (SEQ ID NO. 50), rs17086497 (SEQ ID NO. 51), rs3794395 (SEQ ID NO. 52), rs9554317 (SEQ ID NO. 53), rs9513073 (SEQ ID NO. 54), rs9513074 (SEQ ID NO. 55), rs9508015 (SEQ ID NO. 56), rs2011950 (SEQ ID NO. 57), rs9513110 (SEQ ID NO. 58), rs9513112 (SEQ ID NO. 59), rs9513113 (SEQ ID NO. 60), rs9551471 (SEQ ID NO. 61), rs2296285 (SEQ ID NO. 62), rs9513116 (SEQ ID NO. 63), rs9551473 (SEQ ID NO. 64), rs7330109 (SEQ ID NO. 65), rs9508037 (SEQ ID NO. 66), rs1924981 (SEQ ID NO. 67), rs34140996 (SEQ ID NO. 68), rs7985584 (SEQ ID NO. 69), rs7992940 (SEQ ID NO. 70) and rs718273 (SEQ ID NO. 71).

Accordingly, in an another embodiment of the present invention the one or more variant alleles of the VEGFR-1 gene include variations in the VEGFR-1 gene corresponding to linked SNPs as well as other linked SNPs upstream or downstream from VEGFR-1 gene corresponding to variations in the PAN3 gene and in intergenic DNAselected from the group consisting of:

(a) a sequence corresponding to rs17619037 (SEQ ID NO. 5) or having at least 80%, at least 85%, or more preferably at least 90%, or even more preferably, at least 95%, at least 96%, at least 97%, at least 98%, or at least 99% homology to rs17619037 (SEQ ID NO. 5) having a substitution, deletion or addition of at least one nucleotide corresponding to position 51 of rs17619037 (SEQ ID NO. 5);
(b) a sequence corresponding to rs9579177 (SEQ ID NO. 6) or having at least 80%, at least 85%, or more preferably at least 90%, or even more preferably, at least 95%, at least 96%, at least 97%, at least 98%, or at least 99% homology to rs9579177 (SEQ ID NO. 6) having a substitution, deletion or addition of at least one nucleotide corresponding to position 51 of rs9579177 (SEQ ID NO. 6);
(c) a sequence corresponding to rs9579176 (SEQ ID NO. 7) or having at least 80%, at least 85%, or more preferably at least 90%, or even more preferably, at least 95%, at least 96%, at least 97%, at least 98%, or at least 99% homology to rs9579176 (SEQ ID NO. 7) having a substitution, deletion or addition of at least one nucleotide corresponding to position 51 of rs9579176 (SEQ ID NO. 7);
(d) a sequence corresponding to rs9554319 (SEQ ID NO. 8) or having at least 80%, at least 85%, or more preferably at least 90%, or even more preferably, at least 95%, at least 96%, at least 97%, at least 98%, or at least 99% homology to rs9554319 (SEQ ID NO. 8) having a substitution, deletion or addition of at least one nucleotide corresponding to position 51 of rs9554319 (SEQ ID NO. 8);
(e) a sequence corresponding to rs7996030 (SEQ ID NO. 9) or having at least 80%, at least 85%, or more preferably at least 90%, or even more preferably, at least 95%, at least 96%, at least 97%, at least 98%, or at least 99% homology to rs7996030 (SEQ ID NO. 9) having a substitution, deletion or addition of at least one nucleotide corresponding to position 51 of rs7996030 (SEQ ID NO. 9);
(f) a sequence corresponding to rs9513075 (SEQ ID NO. 10) or having at least 80%, at least 85%, or more preferably at least 90%, or even more preferably, at least 95%, at least 96%, at least 97%, at least 98%, or at least 99% homology to rs9513075 (SEQ ID NO. 10) having a substitution, deletion or addition of at least one nucleotide corresponding to position 51 of rs9513075 (SEQ ID NO. 10);
(g) a sequence corresponding to rs7993418 (SEQ ID NO. 11) or having at least 80%, at least 85%, or more preferably at least 90%, or even more preferably, at least 95%, at least 96%, at least 97%, at least 98%, or at least 99% homology to rs7993418 (SEQ ID NO. 11) having a substitution, deletion or addition of at least one nucleotide corresponding to position 51 of rs7993418 (SEQ ID NO. 11);
(h) a sequence corresponding to rs9513071 (SEQ ID NO. 12) or having at least 80%, at least 85%, or more preferably at least 90%, or even more preferably, at least 95%, at least 96%, at least 97%, at least 98%, or at least 99% homology to rs9513071 (SEQ ID NO. 12) having a substitution, deletion or addition of at least one nucleotide corresponding to position 51 of rs9513071 (SEQ ID NO. 12);
(i) a sequence corresponding to rs11620238 (SEQ ID NO. 13) or having at least 80%, at least 85%, or more preferably at least 90%, or even more preferably, at least 95%, at least 96%, at least 97%, at least 98%, or at least 99% homology to rs11620238 (SEQ ID NO. 13) having a substitution, deletion or addition of at least one nucleotide corresponding to position 51 of rs11620238 (SEQ ID NO. 13);
(j) a sequence corresponding to rs17618631 (SEQ ID NO. 14) or having at least 80%, at least 85%, or more preferably at least 90%, or even more preferably, at least 95%, at least 96%, at least 97%, at least 98%, or at least 99% homology to rs17618631 (SEQ ID NO. 14) having a substitution, deletion or addition of at least one nucleotide corresponding to position 51 of rs17618631 (SEQ ID NO. 14);
(k) a sequence corresponding to rs12429309 (SEQ ID NO. 15) or having at least 80%, at least 85%, or more preferably at least 90%, or even more preferably, at least 95%, at least 96%, at least 97%, at least 98%, or at least 99% homology to rs12429309 (SEQ ID NO. 15) having a substitution, deletion or addition of at least one nucleotide corresponding to position 51 of rs12429309 (SEQ ID NO. 15);
(l) a sequence corresponding to rs7982251 (SEQ ID NO 16) or having at least 80%, at least 85%, or more preferably at least 90%, or even more preferably, at least 95%, at least 96%, at least 97%, at least 98%, or at least 99% homology to rs7982251 (SEQ ID NO 16) having a substitution, deletion or addition of at least one nucleotide corresponding to position 51 of rs7982251 (SEQ ID NO 16);
(m) a sequence corresponding to rs9508016 (SEQ ID NO. 17) or having at least 80%, at least 85%, or more preferably at least 90%, or even more preferably, at least 95%, at least 96%, at least 97%, at least 98%, or at least 99% homology to rs9508016 (SEQ ID NO. 17) having a substitution, deletion or addition of at least one nucleotide corresponding to position 51 of rs9508016 (SEQ ID NO. 17);
(n) a sequence corresponding to rs7982957 (SEQ ID NO. 18) or having at least 80%, at least 85%, or more preferably at least 90%, or even more preferably, at least 95%, at least 96%, at least 97%, at least 98%, or at least 99% homology to rs7982957 (SEQ ID NO. 18) having a substitution, deletion or addition of at least one nucleotide corresponding to position 51 of rs7982957 (SEQ ID NO. 18);
(o) a sequence corresponding to rs3794400 (SEQ ID NO. 19) or having at least 80%, at least 85%, or more preferably at least 90%, or even more preferably, at least 95%, at least 96%, at least 97%, at least 98%, or at least 99% homology to rs3794400 (SEQ ID NO. 19) having a substitution, deletion or addition of at least one nucleotide corresponding to position 51 of rs3794400 (SEQ ID NO. 19);
(p) a sequence corresponding to rs45455097 (SEQ ID NO. 40) or having at least 80%, at least 85%, or more preferably at least 90%, or even more preferably, at least 95%, at least 96%, at least 97%, at least 98%, or at least 99% homology to rs45455097 (SEQ ID NO. 40) having a substitution, deletion or addition of at least one nucleotide corresponding to position 51 of rs45455097 (SEQ ID NO. 40);
(q) a sequence corresponding to rs1886233 (SEQ ID NO. 41) or having at least 80%, at least 85%, or more preferably at least 90%, or even more preferably, at least 95%, at least 96%, at least 97%, at least 98%, or at least 99% homology to rs1886233 (SEQ ID NO. 41) having a substitution, deletion or addition of at least one nucleotide corresponding to position 51 of rs1886233 (SEQ ID NO. 41);
(r) a sequence corresponding to rs9554309 (SEQ ID NO. 42) or having at least 80%, at least 85%, or more preferably at least 90%, or even more preferably, at least 95%, at least 96%, at least 97%, at least 98%, or at least 99% homology to rs9554309 (SEQ ID NO. 42) having a substitution, deletion or addition of at least one nucleotide corresponding to position 51 of rs9554309 (SEQ ID NO. 42);
(s) a sequence corresponding to rs11619230 (SEQ ID NO. 43) or having at least 80%, at least 85%, or more preferably at least 90%, or even more preferably, at least 95%, at least 96%, at least 97%, at least 98%, or at least 99% homology to rs11619230 (SEQ ID NO. 43) having a substitution, deletion or addition of at least one nucleotide corresponding to position 51 of rs11619230 (SEQ ID NO. 43);
(t) a sequence corresponding to rs9554311 (SEQ ID NO. 44) or having at least 80%, at least 85%, or more preferably at least 90%, or even more preferably, at least 95%, at least 96%, at least 97%, at least 98%, or at least 99% homology to rs9554311 (SEQ ID NO. 44) having a substitution, deletion or addition of at least one nucleotide corresponding to position 51 of rs9554311 (SEQ ID NO. 44);
(u) a sequence corresponding to rs4771233 (SEQ ID NO. 45) or having at least 80%, at least 85%, or more preferably at least 90%, or even more preferably, at least 95%, at least 96%, at least 97%, at least 98%, or at least 99% homology to rs4771233 (SEQ ID NO. 45) having a substitution, deletion or addition of at least one nucleotide corresponding to position 51 of rs4771233 (SEQ ID NO. 45);
(v) a sequence corresponding to rs6491274 (SEQ ID NO. 46) or having at least 80%, at least 85%, or more preferably at least 90%, or even more preferably, at least 95%, at least 96%, at least 97%, at least 98%, or at least 99% homology to rs6491274 (SEQ ID NO. 46) having a substitution, deletion or addition of at least one nucleotide corresponding to position 51 of rs6491274 (SEQ ID NO. 46);
(w) a sequence corresponding to rs7982639 (SEQ ID NO. 47) or having at least 80%, at least 85%, or more preferably at least 90%, or even more preferably, at least 95%, at least 96%, at least 97%, at least 98%, or at least 99% homology to rs7982639 (SEQ ID NO. 47) having a substitution, deletion or addition of at least one nucleotide corresponding to position 51 of rs7982639 (SEQ ID NO. 47);
(x) a sequence corresponding to rs12877718 (SEQ ID NO. 48) or having at least 80%, at least 85%, or more preferably at least 90%, or even more preferably, at least 95%, at least 96%, at least 97%, at least 98%, or at least 99% homology to rs12877718 (SEQ ID NO. 48) having a substitution, deletion or addition of at least one nucleotide corresponding to position 51 of rs12877718 (SEQ ID NO. 48);
(y) a sequence corresponding to rs10507382 (SEQ ID NO. 49) or having at least 80%, at least 85%, or more preferably at least 90%, or even more preferably, at least 95%, at least 96%, at least 97%, at least 98%, or at least 99% homology to rs10507382 (SEQ ID NO. 49) having a substitution, deletion or addition of at least one nucleotide corresponding to position 51 of rs10507382 (SEQ ID NO. 49);
(z) a sequence corresponding to rs57354941 (SEQ ID NO. 50) or having at least 80%, at least 85%, or more preferably at least 90%, or even more preferably, at least 95%, at least 96%, at least 97%, at least 98%, or at least 99% homology to rs57354941 (SEQ ID NO. 50) having a substitution, deletion or addition of at least one nucleotide corresponding to position 51 of rs57354941 (SEQ ID NO. 50);
(aa) a sequence corresponding to rs17086497 (SEQ ID NO. 51) or having at least 80%, at least 85%, or more preferably at least 90%, or even more preferably, at least 95%, at least 96%, at least 97%, at least 98%, or at least 99% homology to rs17086497 (SEQ ID NO. 51) having a substitution, deletion or addition of at least one nucleotide corresponding to position 51 of rs17086497 (SEQ ID NO. 51);
(bb) a sequence corresponding to rs3794395 (SEQ ID NO. 52) or having at least 80%, at least 85%, or more preferably at least 90%, or even more preferably, at least 95%, at least 96%, at least 97%, at least 98%, or at least 99% homology to rs3794395 (SEQ ID NO. 52) having a substitution, deletion or addition of at least one nucleotide corresponding to position 51 of rs3794395 (SEQ ID NO. 52);
(cc) a sequence corresponding to rs9554317 (SEQ ID NO. 53) or having at least 80%, at least 85%, or more preferably at least 90%, or even more preferably, at least 95%, at least 96%, at least 97%, at least 98%, or at least 99% homology to rs9554317 (SEQ ID NO. 53) having a substitution, deletion or addition of at least one nucleotide corresponding to position 51 of rs9554317 (SEQ ID NO. 53);
(dd) a sequence corresponding to rs9513073 (SEQ ID NO. 54) or having at least 80%, at least 85%, or more preferably at least 90%, or even more preferably, at least 95%, at least 96%, at least 97%, at least 98%, or at least 99% homology to rs9513073 (SEQ ID NO. 54) having a substitution, deletion or addition of at least one nucleotide corresponding to position 51 of rs9513073 (SEQ ID NO. 54);
(ee) a sequence corresponding to rs9513074 (SEQ ID NO. 55) or having at least 80%, at least 85%, or more preferably at least 90%, or even more preferably, at least 95%, at least 96%, at least 97%, at least 98%, or at least 99% homology to rs9513074 (SEQ ID NO. 55) having a substitution, deletion or addition of at least one nucleotide corresponding to position 51 of rs9513074 (SEQ ID NO. 55);
(ff) a sequence corresponding to rs9508015 (SEQ ID NO. 56) or having at least 80%, at least 85%, or more preferably at least 90%, or even more preferably, at least 95%, at least 96%, at least 97%, at least 98%, or at least 99% homology to rs9508015 (SEQ ID NO. 56) having a substitution, deletion or addition of at least one nucleotide corresponding to position 51 of rs9508015 (SEQ ID NO. 56);
(gg) a sequence corresponding to rs2011950 (SEQ ID NO. 57) or having at least 80%, at least 85%, or more preferably at least 90%, or even more preferably, at least 95%, at least 96%, at least 97%, at least 98%, or at least 99% homology to rs2011950 (SEQ ID NO. 57) having a substitution, deletion or addition of at least one nucleotide corresponding to position 51 of rs2011950 (SEQ ID NO. 57);
(hh) a sequence corresponding to rs9513110 (SEQ ID NO. 58) or having at least 80%, at least 85%, or more preferably at least 90%, or even more preferably, at least 95%, at least 96%, at least 97%, at least 98%, or at least 99% homology to rs9513110 (SEQ ID NO. 58) having a substitution, deletion or addition of at least one nucleotide corresponding to position 51 of rs9513110 (SEQ ID NO. 58);
(ii) a sequence corresponding to rs9513112 (SEQ ID NO. 59) or having at least 80%, at least 85%, or more preferably at least 90%, or even more preferably, at least 95%, at least 96%, at least 97%, at least 98%, or at least 99% homology to rs9513112 (SEQ ID NO. 59) having a substitution, deletion or addition of at least one nucleotide corresponding to position 51 of rs9513112 (SEQ ID NO. 59);
(jj) a sequence corresponding to rs9513113 (SEQ ID NO. 60) or having at least 80%, at least 85%, or more preferably at least 90%, or even more preferably, at least 95%, at least 96%, at least 97%, at least 98%, or at least 99% homology to rs9513113 (SEQ ID NO. 60) having a substitution, deletion or addition of at least one nucleotide corresponding to position 51 of rs9513113 (SEQ ID NO. 60);
(kk) a sequence corresponding to rs9551471 (SEQ ID NO. 61) or having at least 80%, at least 85%, or more preferably at least 90%, or even more preferably, at least 95%, at least 96%, at least 97%, at least 98%, or at least 99% homology to rs9551471 (SEQ ID NO. 61) having a substitution, deletion or addition of at least one nucleotide corresponding to position 51 of rs9551471 (SEQ ID NO. 61);
(ll) a sequence corresponding to rs2296285 (SEQ ID NO. 62) or having at least 80%, at least 85%, or more preferably at least 90%, or even more preferably, at least 95%, at least 96%, at least 97%, at least 98%, or at least 99% homology to rs2296285 (SEQ ID NO. 62) having a substitution, deletion or addition of at least one nucleotide corresponding to position 51 of rs2296285 (SEQ ID NO. 62);
(mm) a sequence corresponding to rs9513116 (SEQ ID NO. 63) or having at least 80%, at least 85%, or more preferably at least 90%, or even more preferably, at least 95%, at least 96%, at least 97%, at least 98%, or at least 99% homology rs9513116 (SEQ ID NO. 63) having a substitution, deletion or addition of at least one nucleotide corresponding to position 51 of rs9513116 (SEQ ID NO. 63);
(nn) a sequence corresponding to rs9551473 (SEQ ID NO. 64) or having at least 80%, at least 85%, or more preferably at least 90%, or even more preferably, at least 95%, at least 96%, at least 97%, at least 98%, or at least 99% homology to rs9551473 (SEQ ID NO. 64) having a substitution, deletion or addition of at least one nucleotide corresponding to position 51 of rs9551473 (SEQ ID NO. 64);
(oo) a sequence corresponding to rs7330109 (SEQ ID NO. 65) or having at least 80%, at least 85%, or more preferably at least 90%, or even more preferably, at least 95%, at least 96%, at least 97%, at least 98%, or at least 99% homology to rs7330109 (SEQ ID NO. 65) having a substitution, deletion or addition of at least one nucleotide corresponding to position 51 of rs7330109 (SEQ ID NO. 65);
(pp) a sequence corresponding to rs9508037 (SEQ ID NO. 66) or having at least 80%, at least 85%, or more preferably at least 90%, or even more preferably, at least 95%, at least 96%, at least 97%, at least 98%, or at least 99% homology to rs9508037 (SEQ ID NO. 66) having a substitution, deletion or addition of at least one nucleotide corresponding to position 51 of rs9508037 (SEQ ID NO. 66);
(qq) a sequence corresponding to rs1924981 (SEQ ID NO. 67) or having at least 80%, at least 85%, or more preferably at least 90%, or even more preferably, at least 95%, at least 96%, at least 97%, at least 98%, or at least 99% homology to rs1924981 (SEQ ID NO. 67) having a substitution, deletion or addition of at least one nucleotide corresponding to position 51 of rs1924981 (SEQ ID NO. 67);
(rr) a sequence corresponding to rs34140996 (SEQ ID NO. 68) or having at least 80%, at least 85%, or more preferably at least 90%, or even more preferably, at least 95%, at least 96%, at least 97%, at least 98%, or at least 99% homology to rs3794400 rs34140996 (SEQ ID NO. 68) having a substitution, deletion or addition of at least one nucleotide corresponding to position 51 of rs34140996 (SEQ ID NO. 68);
(ss) a sequence corresponding to rs7985584 (SEQ ID NO. 69) or having at least 80%, at least 85%, or more preferably at least 90%, or even more preferably, at least 95%, at least 96%, at least 97%, at least 98%, or at least 99% homology to rs7985584 (SEQ ID NO. 69) having a substitution, deletion or addition of at least one nucleotide corresponding to position 51 of rs7985584 (SEQ ID NO. 69);
(tt) a sequence corresponding to rs7992940 (SEQ ID NO. 70) or having at least 80%, at least 85%, or more preferably at least 90%, or even more preferably, at least 95%, at least 96%, at least 97%, at least 98%, or at least 99% homology to rs7992940 (SEQ ID NO. 70) having a substitution, deletion or addition of at least one nucleotide corresponding to position 51 of rs7992940 (SEQ ID NO. 70); and
(uu) a sequence corresponding to rs718273 (SEQ ID NO. 71) or having at least 80%, at least 85%, or more preferably at least 90%, or even more preferably, at least 95%, at least 96%, at least 97%, at least 98%, or at least 99% homology to rs718273 (SEQ ID NO. 71) having a substitution, deletion or addition of at least one nucleotide corresponding to position 51 of rs718273 (SEQ ID NO. 71).

In the context of the herein described invention, the above described homology, with regard to the identified SNPs, applies to each embodiment herein described wherein specific SNPs are recited.

Further, the variations can include a substitution(s), deletion(s) or addition(s) at any position from position 1 to 50 and 52 to 101 of rs17619037 (SEQ ID NO. 5), rs9579177 (SEQ ID NO. 6), rs9579176 (SEQ ID NO. 7), rs9554319 (SEQ ID NO. 8), rs7996030 (SEQ ID NO. 9), rs9513075 (SEQ ID NO. 10), rs7993418 (SEQ ID NO. 11), rs9513071 (SEQ ID NO. 12), rs11620238 (SEQ ID NO. 13), rs17618631 (SEQ ID NO. 14), rs12429309 (SEQ ID NO. 15), rs7982251 (SEQ ID NO 16), rs9508016 (SEQ ID NO. 17), rs7982957 (SEQ ID NO. 18), rs3794400 (SEQ ID NO. 19), rs45455097 (SEQ ID NO. 40), rs1886233 (SEQ ID NO. 41), rs9554309 (SEQ ID NO. 42), rs11619230 (SEQ ID NO. 43), rs9554311 (SEQ ID NO. 44), rs4771233 (SEQ ID NO. 45), rs6491274 (SEQ ID NO. 46), rs7982639 (SEQ ID NO. 47), rs12877718 (SEQ ID NO. 48), rs10507382 (SEQ ID NO. 49), rs57354941 (SEQ ID NO. 50), rs17086497 (SEQ ID NO. 51), rs3794395 (SEQ ID NO. 52), rs9554317 (SEQ ID NO. 53), rs9513073 (SEQ ID NO. 54), rs9513074 (SEQ ID NO. 55), rs9508015 (SEQ ID NO. 56), rs2011950 (SEQ ID NO. 57), rs9513110 (SEQ ID NO. 58), rs9513112 (SEQ ID NO. 59), rs9513113 (SEQ ID NO. 60), rs9551471 (SEQ ID NO. 61), rs2296285 (SEQ ID NO. 62), rs9513116 (SEQ ID NO. 63), rs9551473 (SEQ ID NO. 64), rs7330109 (SEQ ID NO. 65), rs9508037 (SEQ ID NO. 66), rs1924981 (SEQ ID NO. 67), rs34140996 (SEQ ID NO. 68), rs7985584 (SEQ ID NO. 69), rs7992940 (SEQ ID NO. 70) and rs718273 (SEQ ID NO. 71).

Accordingly, in another embodiment of the present invention the one or more variant alleles of the VEGFR-1 gene include variations in the VEGFR-1 gene corresponding to linked SNPs as well as other linked SNPs upstream or downstream from VEGFR-1 gene corresponding to variations in the PAN3 gene and in intergenic DNA selected from the group consisting of:

(a) a sequence corresponding to rs17619037 (SEQ ID NO. 5) or having at least 80%, at least 85%, or more preferably at least 90%, or even more preferably, at least 95%, at least 96%, at least 97%, at least 98%, or at least 99% homology to rs17619037 (SEQ ID NO. 5), wherein position 51 is an A or G and having a substitution, deletion or addition of at least one nucleotide at any position from position 1 to 50 and 52 to 101;
(b) a sequence corresponding to rs9579177 (SEQ ID NO. 6) or having at least 80%, at least 85%, or more preferably at least 90%, or even more preferably, at least 95%, at least 96%, at least 97%, at least 98%, or at least 99% to rs9579177 (SEQ ID NO. 6), wherein position 51 is an A or G and having a substitution, deletion or addition of at least one nucleotide at any position from position 1 to 50 and 52 to 101;
(c) a sequence corresponding to rs9579176 (SEQ ID NO. 7) or having at least 80%, at least 85%, or more preferably at least 90%, or even more preferably, at least 95%, at least 96%, at least 97%, at least 98%, or at least 99% homology to rs9579176 (SEQ ID NO. 7), wherein position 51 is an A or G and having a substitution, deletion or addition of at least one nucleotide at any position from position 1 to 50 and 52 to 101;
(d) a sequence corresponding to rs9554319 (SEQ ID NO. 8) or having at least 80%, at least 85%, or more preferably at least 90%, or even more preferably, at least 95%, at least 96%, at least 97%, at least 98%, or at least 99% homology to rs9554319 (SEQ ID NO. 8), wherein position 51 is a C or T and having a substitution, deletion or addition of at least one nucleotide at any position from position 1 to 50 and 52 to 101;
(e) a sequence corresponding to rs7996030 (SEQ ID NO. 9) or having at least 80%, at least 85%, or more preferably at least 90%, or even more preferably, at least 95%, at least 96%, at least 97%, at least 98%, or at least 99% homology to rs7996030 (SEQ ID NO. 9), wherein position 51 is an A or G and having a substitution, deletion or addition of at least one nucleotide at any position from position 1 to 50 and 52 to 101;
(f) a sequence corresponding to rs9513075 (SEQ ID NO. 10) or having at least 80%, at least 85%, or more preferably at least 90%, or even more preferably, at least 95%, at least 96%, at least 97%, at least 98%, or at least 99% homology to rs9513075 (SEQ ID NO. 10), wherein position 51 is an A or G and having a substitution, deletion or addition of at least one nucleotide at any position from position 1 to 50 and 52 to 101;
(g) a sequence corresponding to rs7993418 (SEQ ID NO. 11) or having at least 80%, at least 85%, or more preferably at least 90%, or even more preferably, at least 95%, at least 96%, at least 97%, at least 98%, or at least 99% homology to rs7993418 (SEQ ID NO. 11), wherein position 51 is an A or G and having a substitution, deletion or addition of at least one nucleotide at any position from position 1 to 50 and 52 to 101;
(h) a sequence corresponding to rs9513071 (SEQ ID NO. 12) or having at least 80%, at least 85%, or more preferably at least 90%, or even more preferably, at least 95%, at least 96%, at least 97%, at least 98%, or at least 99% homology to rs9513071 (SEQ ID NO. 12), wherein position 51 is a T or G and having a substitution, deletion or addition of at least one nucleotide at any position from position 1 to 50 and 52 to 101;
(i) a sequence corresponding to rs11620238 (SEQ ID NO. 13) or having at least 80%, at least 85%, or more preferably at least 90%, or even more preferably, at least 95%, at least 96%, at least 97%, at least 98%, or at least 99% homology to rs11620238 (SEQ ID NO. 13), wherein position 51 is a T or G and having a substitution, deletion or addition of at least one nucleotide at any position from position 1 to 50 and 52 to 101;
(j) a sequence corresponding to rs17618631 (SEQ ID NO. 14) or having at least 80%, at least 85%, or more preferably at least 90%, or even more preferably, at least 95%, at least 96%, at least 97%, at least 98%, or at least 99% homology to rs17618631 (SEQ ID NO. 14), wherein position 51 is a C or G and having a substitution, deletion or addition of at least one nucleotide at any position from position 1 to 50 and 52 to 101;
(k) a sequence corresponding to rs12429309 (SEQ ID NO. 15) or having at least 80%, at least 85%, or more preferably at least 90%, or even more preferably, at least 95%, at least 96%, at least 97%, at least 98%, or at least 99% homology to rs12429309 (SEQ ID NO. 15), wherein position 51 is a T or C and having a substitution, deletion or addition of at least one nucleotide at any position from position 1 to 50 and 52 to 101;
(l) a sequence corresponding to rs7982251 (SEQ ID NO 16) or having at least 80%, at least 85%, or more preferably at least 90%, or even more preferably, at least 95%, at least 96%, at least 97%, at least 98%, or at least 99% homology to rs7982251 (SEQ ID NO 16), wherein position 51 is a T or C and having a substitution, deletion or addition of at least one nucleotide at any position from position 1 to 50 and 52 to 101;
(m) a sequence corresponding to rs9508016 (SEQ ID NO. 17) or having at least 80%, at least 85%, or more preferably at least 90%, or even more preferably, at least 95%, at least 96%, at least 97%, at least 98%, or at least 99% homology to rs9508016 (SEQ ID NO. 17), wherein position 51 is an A or G and having a substitution, deletion or addition of at least one nucleotide at any position from position 1 to 50 and 52 to 101;
(n) a sequence corresponding to rs7982957 (SEQ ID NO. 18) or having at least 80%, at least 85%, or more preferably at least 90%, or even more preferably, at least 95%, at least 96%, at least 97%, at least 98%, or at least 99% homology to rs7982957 (SEQ ID NO. 18), wherein position 51 is an A or T and having a substitution, deletion or addition of at least one nucleotide at any position from position 1 to 50 and 52 to 101;
(o) a sequence corresponding to rs3794400 (SEQ ID NO. 19) or having at least 80%, at least 85%, or more preferably at least 90%, or even more preferably, at least 95%, at least 96%, at least 97%, at least 98%, or at least 99% homology to rs3794400 (SEQ ID NO. 19), wherein position 51 is a T or C and having a substitution, deletion or addition of at least one nucleotide at any position from position 1 to 50 and 52 to 101;
(p) a sequence corresponding to rs45455097 (SEQ ID NO. 40) or having at least 80%, at least 85%, or more preferably at least 90%, or even more preferably, at least 95%, at least 96%, at least 97%, at least 98%, or at least 99% homology to rs45455097 (SEQ ID NO. 40), wherein position 51 is a T or C and having a substitution, deletion or addition of at least one nucleotide at any position from position 1 to 50 and 52 to 101;
(q) a sequence corresponding to rs1886233 (SEQ ID NO. 41) or having at least 80%, at least 85%, or more preferably at least 90%, or even more preferably, at least 95%, at least 96%, at least 97%, at least 98%, or at least 99% homology to rs1886233 (SEQ ID NO. 41), wherein position 51 is an A or G and having a substitution, deletion or addition of at least one nucleotide at any position from position 1 to 50 and 52 to 101;
(r) a sequence corresponding to rs9554309 (SEQ ID NO. 42) or having at least 80%, at least 85%, or more preferably at least 90%, or even more preferably, at least 95%, at least 96%, at least 97%, at least 98%, or at least 99% homology to rs9554309 (SEQ ID NO. 42), wherein position 51 is a T or C and having a substitution, deletion or addition of at least one nucleotide at any position from position 1 to 50 and 52 to 101;
(s) a sequence corresponding to rs11619230 (SEQ ID NO. 43) or having at least 80%, at least 85%, or more preferably at least 90%, or even more preferably, at least 95%, at least 96%, at least 97%, at least 98%, or at least 99% homology to rs11619230 (SEQ ID NO. 43), wherein position 51 is a T or C and having a substitution, deletion or addition of at least one nucleotide at any position from position 1 to 50 and 52 to 101;
(t) a sequence corresponding to rs9554311 (SEQ ID NO. 44) or having at least 80%, at least 85%, or more preferably at least 90%, or even more preferably, at least 95%, at least 96%, at least 97%, at least 98%, or at least 99% homology to rs9554311 (SEQ ID NO. 44), wherein position 51 is a T or C and having a substitution, deletion or addition of at least one nucleotide at any position from position 1 to 50 and 52 to 101;
(u) a sequence corresponding to rs4771233 (SEQ ID NO. 45) or having at least 80%, at least 85%, or more preferably at least 90%, or even more preferably, at least 95%, at least 96%, at least 97%, at least 98%, or at least 99% homology to rs4771233 (SEQ ID NO. 45), wherein position 51 is a T or C and having a substitution, deletion or addition of at least one nucleotide at any position from position 1 to 50 and 52 to 101;
(v) a sequence corresponding to rs6491274 (SEQ ID NO. 46) or having at least 80%, at least 85%, or more preferably at least 90%, or even more preferably, at least 95%, at least 96%, at least 97%, at least 98%, or at least 99% homology to rs6491274 (SEQ ID NO. 46), wherein position 51 is a T or C and having a substitution, deletion or addition of at least one nucleotide at any position from position 1 to 50 and 52 to 101;
(w) a sequence corresponding to rs7982639 (SEQ ID NO. 47) or having at least 80%, at least 85%, or more preferably at least 90%, or even more preferably, at least 95%, at least 96%, at least 97%, at least 98%, or at least 99% homology to rs7982639 (SEQ ID NO. 47), wherein position 51 is an A or G and having a substitution, deletion or addition of at least one nucleotide at any position from position 1 to 50 and 52 to 101;
(x) a sequence corresponding to rs12877718 (SEQ ID NO. 48) or having at least 80%, at least 85%, or more preferably at least 90%, or even more preferably, at least 95%, at least 96%, at least 97%, at least 98%, or at least 99% homology to rs12877718 (SEQ ID NO. 48), wherein position 51 is a T or C and having a substitution, deletion or addition of at least one nucleotide at any position from position 1 to 50 and 52 to 101;
(y) a sequence corresponding to rs10507382 (SEQ ID NO. 49) or having at least 80%, at least 85%, or more preferably at least 90%, or even more preferably, at least 95%, at least 96%, at least 97%, at least 98%, or at least 99% homology to rs10507382 (SEQ ID NO. 49), wherein position 51 is a T or C and having a substitution, deletion or addition of at least one nucleotide at any position from position 1 to 50 and 52 to 101;
(z) a sequence corresponding to rs57354941 (SEQ ID NO. 50) or having at least 80%, at least 85%, or more preferably at least 90%, or even more preferably, at least 95%, at least 96%, at least 97%, at least 98%, or at least 99% homology to rs57354941 (SEQ ID NO. 50), wherein position 51 is a T or C and having a substitution, deletion or addition of at least one nucleotide at any position from position 1 to 50 and 52 to 101;
(aa) a sequence corresponding to rs17086497 (SEQ ID NO. 51) or having at least 80%, at least 85%, or more preferably at least 90%, or even more preferably, at least 95%, at least 96%, at least 97%, at least 98%, or at least 99% homology to rs17086497 (SEQ ID NO. 51), wherein position 51 is a C or A and having a substitution, deletion or addition of at least one nucleotide at any position from position 1 to 50 and 52 to 101;
(bb) a sequence corresponding to rs3794395 (SEQ ID NO. 52) or having at least 80%, at least 85%, or more preferably at least 90%, or even more preferably, at least 95%, at least 96%, at least 97%, at least 98%, or at least 99% homology to rs3794395 (SEQ ID NO. 52), wherein position 51 is a T or C and having a substitution, deletion or addition of at least one nucleotide at any position from position 1 to 50 and 52 to 101;
(cc) a sequence corresponding to rs9554317 (SEQ ID NO. 53) or having at least 80%, at least 85%, or more preferably at least 90%, or even more preferably, at least 95%, at least 96%, at least 97%, at least 98%, or at least 99% homology to rs9554317 (SEQ ID NO. 53), wherein position 51 is a T or C and having a substitution, deletion or addition of at least one nucleotide at any position from position 1 to 50 and 52 to 101;
(dd) a sequence corresponding to rs9513073 (SEQ ID NO. 54) or having at least 80%, at least 85%, or more preferably at least 90%, or even more preferably, at least 95%, at least 96%, at least 97%, at least 98%, or at least 99% homology to rs9513073 (SEQ ID NO. 54), wherein position 51 is an A or G and having a substitution, deletion or addition of at least one nucleotide at any position from position 1 to 50 and 52 to 101;
(ee) a sequence corresponding to rs9513074 (SEQ ID NO. 55) or having at least 80%, at least 85%, or more preferably at least 90%, or even more preferably, at least 95%, at least 96%, at least 97%, at least 98%, or at least 99% homology to rs9513074 (SEQ ID NO. 55), wherein position 51 is a T or C and having a substitution, deletion or addition of at least one nucleotide at any position from position 1 to 50 and 52 to 101;
(ff) a sequence corresponding to rs9508015 (SEQ ID NO. 56) or having at least 80%, at least 85%, or more preferably at least 90%, or even more preferably, at least 95%, at least 96%, at least 97%, at least 98%, or at least 99% homology to rs9508015 (SEQ ID NO. 56), wherein position 51 is a T or C and having a substitution, deletion or addition of at least one nucleotide at any position from position 1 to 50 and 52 to 101;
(gg) a sequence corresponding to rs2011950 (SEQ ID NO. 57) or having at least 80%, at least 85%, or more preferably at least 90%, or even more preferably, at least 95%, at least 96%, at least 97%, at least 98%, or at least 99% homology to rs2011950 (SEQ ID NO. 57), wherein position 51 is a G or A and having a substitution, deletion or addition of at least one nucleotide at any position from position 1 to 50 and 52 to 101;
(hh) a sequence corresponding to rs9513110 (SEQ ID NO. 58) or having at least 80%, at least 85%, or more preferably at least 90%, or even more preferably, at least 95%, at least 96%, at least 97%, at least 98%, or at least 99% homology to rs9513110 (SEQ ID NO. 58), wherein position 51 is a T or G and having a substitution, deletion or addition of at least one nucleotide at any position from position 1 to 50 and 52 to 101;
(ii) a sequence corresponding to rs9513112 (SEQ ID NO. 59) or having at least 80%, at least 85%, or more preferably at least 90%, or even more preferably, at least 95%, at least 96%, at least 97%, at least 98%, or at least 99% homology to rs9513112 (SEQ ID NO. 59), wherein position 51 is a G or A and having a substitution, deletion or addition of at least one nucleotide at any position from position 1 to 50 and 52 to 101;
(jj) a sequence corresponding to rs9513113 (SEQ ID NO. 60) or having at least 80%, at least 85%, or more preferably at least 90%, or even more preferably, at least 95%, at least 96%, at least 97%, at least 98%, or at least 99% homology to rs9513113 (SEQ ID NO. 60), wherein position 51 is a T or C and having a substitution, deletion or addition of at least one nucleotide at any position from position 1 to 50 and 52 to 101;
(kk) a sequence corresponding to rs9551471 (SEQ ID NO. 61) or having at least 80%, at least 85%, or more preferably at least 90%, or even more preferably, at least 95%, at least 96%, at least 97%, at least 98%, or at least 99% homology to rs9551471 (SEQ ID NO. 61), wherein position 51 is a G or A and having a substitution, deletion or addition of at least one nucleotide at any position from position 1 to 50 and 52 to 101;
(ll) a sequence corresponding to rs2296285 (SEQ ID NO. 62) or having at least 80%, at least 85%, or more preferably at least 90%, or even more preferably, at least 95%, at least 96%, at least 97%, at least 98%, or at least 99% homology to rs2296285 (SEQ ID NO. 62), wherein position 51 is a T or A and having a substitution, deletion or addition of at least one nucleotide at any position from position 1 to 50 and 52 to 101;
(mm) a sequence corresponding to rs9513116 (SEQ ID NO. 63) or having at least 80%, at least 85%, or more preferably at least 90%, or even more preferably, at least 95%, at least 96%, at least 97%, at least 98%, or at least 99% homology rs9513116 (SEQ ID NO. 63), wherein position 51 is a G or A and having a substitution, deletion or addition of at least one nucleotide at any position from position 1 to 50 and 52 to 101;
(nn) a sequence corresponding to rs9551473 (SEQ ID NO. 64) or having at least 80%, at least 85%, or more preferably at least 90%, or even more preferably, at least 95%, at least 96%, at least 97%, at least 98%, or at least 99% homology to rs9551473 (SEQ ID NO. 64), wherein position 51 is a T or G and having a substitution, deletion or addition of at least one nucleotide at any position from position 1 to 50 and 52 to 101;
(oo) a sequence corresponding to rs7330109 (SEQ ID NO. 65) or having at least 80%, at least 85%, or more preferably at least 90%, or even more preferably, at least 95%, at least 96%, at least 97%, at least 98%, or at least 99% homology to rs7330109 (SEQ ID NO. 65), wherein position 51 is a T or A and having a substitution, deletion or addition of at least one nucleotide at any position from position 1 to 50 and 52 to 101;
(pp) a sequence corresponding to rs9508037 (SEQ ID NO. 66) or having at least 80%, at least 85%, or more preferably at least 90%, or even more preferably, at least 95%, at least 96%, at least 97%, at least 98%, or at least 99% homology to rs9508037 (SEQ ID NO. 66), wherein position 51 is a G or A and having a substitution, deletion or addition of at least one nucleotide at any position from position 1 to 50 and 52 to 101 having a substitution, deletion or addition of at least one nucleotide corresponding to position 51 of rs9508037 (SEQ ID NO. 66);
(qq) a sequence corresponding to rs1924981 (SEQ ID NO. 67) or having at least 80%, at least 85%, or more preferably at least 90%, or even more preferably, at least 95%, at least 96%, at least 97%, at least 98%, or at least 99% homology to rs1924981 (SEQ ID NO. 67), wherein position 51 is a T or C and having a substitution, deletion or addition of at least one nucleotide at any position from position 1 to 50 and 52 to 101;
(rr) a sequence corresponding to rs34140996 (SEQ ID NO. 68) or having at least 80%, at least 85%, or more preferably at least 90%, or even more preferably, at least 95%, at least 96%, at least 97%, at least 98%, or at least 99% homology to rs34140996 (SEQ ID NO. 68), wherein position 51 is a G or A and having a substitution, deletion or addition of at least one nucleotide at any position from position 1 to 50 and 52 to 101;
(ss) a sequence corresponding to rs7985584 (SEQ ID NO. 69) or having at least 80%, at least 85%, or more preferably at least 90%, or even more preferably, at least 95%, at least 96%, at least 97%, at least 98%, or at least 99% homology to rs7985584 (SEQ ID NO. 69), wherein position 51 is a G or A and having a substitution, deletion or addition of at least one nucleotide at any position from position 1 to 50 and 52 to 101;
(tt) a sequence corresponding to rs7992940 (SEQ ID NO. 70) or having at least 80%, at least 85%, or more preferably at least 90%, or even more preferably, at least 95%, at least 96%, at least 97%, at least 98%, or at least 99% homology to rs7992940 (SEQ ID NO. 70), wherein position 51 is a T or G and having a substitution, deletion or addition of at least one nucleotide at any position from position 1 to 50 and 52 to 101; and
(uu) a sequence corresponding to rs718273 (SEQ ID NO. 71) or having at least 80%, at least 85%, or more preferably at least 90%, or even more preferably, at least 95%, at least 96%, at least 97%, at least 98%, or at least 99% homology to rs718273 (SEQ ID NO. 71), wherein position 51 is a T or C and having a substitution, deletion or addition of at least one nucleotide at any position from position 1 to 50 and 52 to 101.

In the context of the herein described invention, the above described homology, with regard to the identified SNPs, applies to each embodiment herein described wherein specific SNPs are recited.

In the context of the present invention, “homology” is understood to refer in the context of “variant alleles” to a sequence identity of at least 80%, particularly an identity of at least 85%, preferably at least 90% and still more preferably at least 95% over the full length of the sequence as defined by the SEQ ID NOs provided herein. In the context of this invention, a skilled person would understand that homology covers further variation(s) in the “variant alleles” in different ethnic groups.

As documented in the appended examples, it could surprisingly be shown that the biomarkers as provided herein and/or the assessment of the individual genetic setting of a given subject in the VEGF-R1 gene are not only associated with overall survival but also risk of progression in patients treated with angiogenesis inhibitors, like bevacizumab.

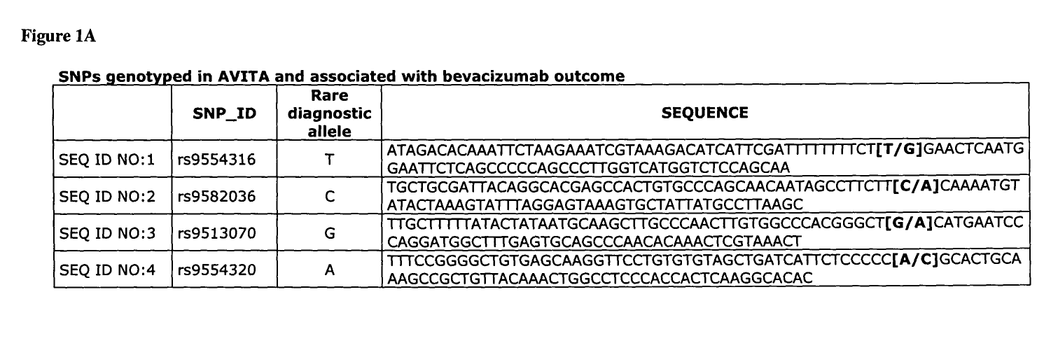

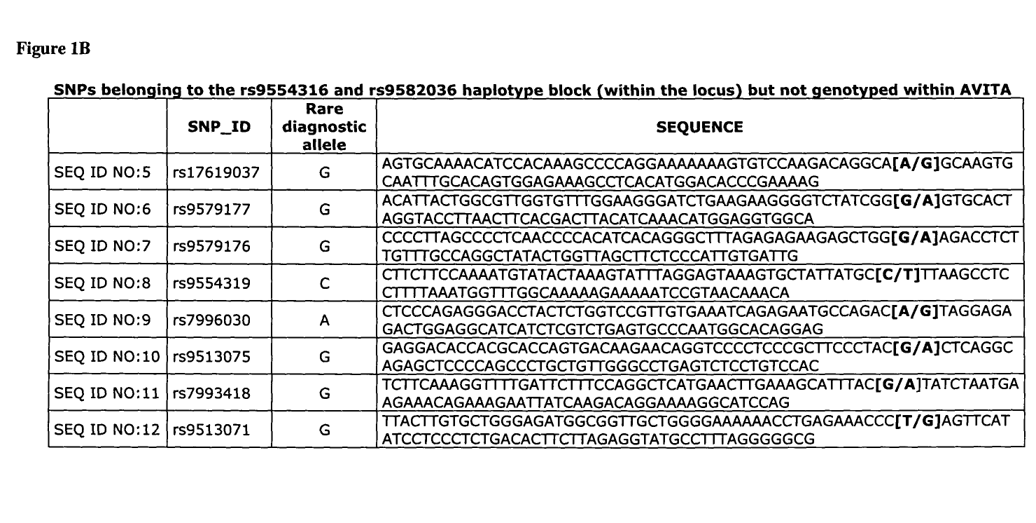

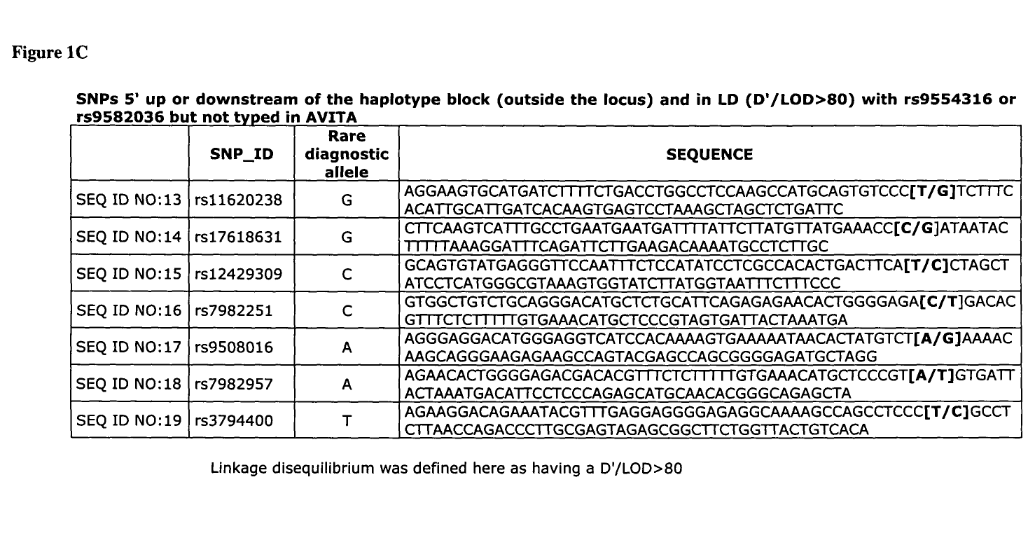

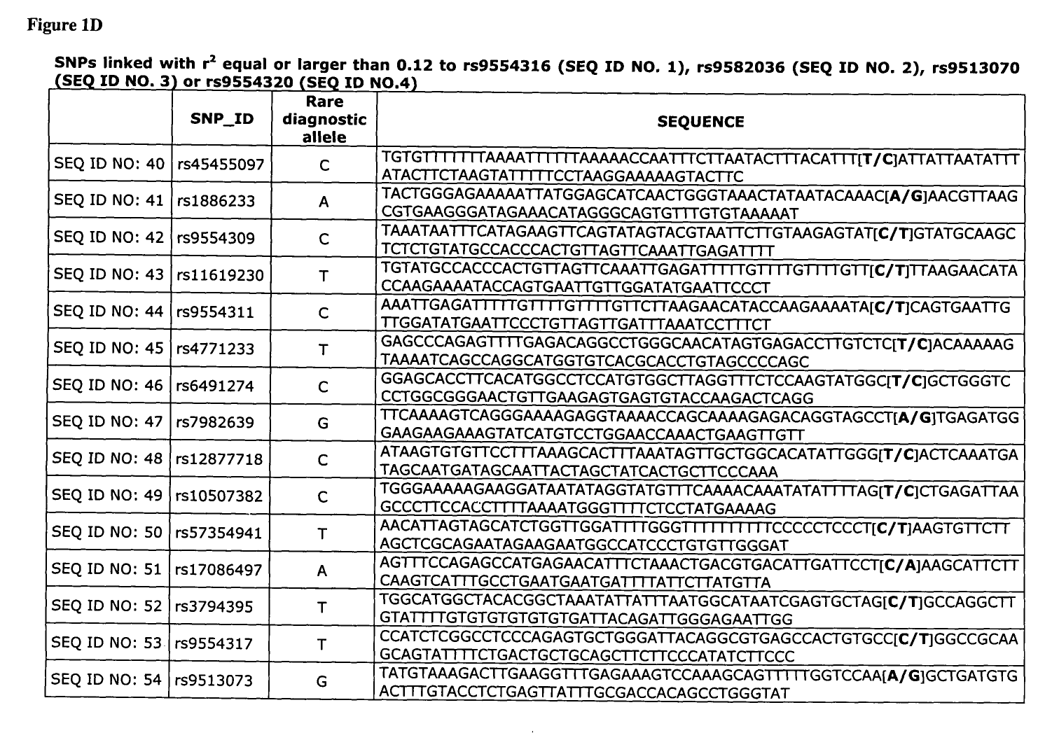

In accordance with the methods of the present invention, four SNPs in the VEGFR-1 gene correlated with overall survival in the bevacizumab-treated group in a study treating metastatic pancreatic cancer with gemcitabine-erlotinib plus bevacizumab or placebo. The four SNPs identified are derived for SNPs rs9582036 (SEQ ID NO. 2), rs9554316 (SEQ ID NO. 1), rs9513070 (SEQ ID NO. 3) and rs9554320 (SEQ ID NO.4). The most significant SNP, rs9582036 (SEQ ID NO. 2), sequence shown in FIG. 1, had p-value<=3e-4 in an allelic risk effect model. Relative to AA carriers, the hazard ratio was 2.0 (CI=1.2-3.4; p=0.009) and 4.7 (CI=2.1-10.7; p=0.0002) for AC and CC carriers, respectively. Median overall survival was increased from 4.8 months in CC (n=9) carriers to 6.0 and 10.3 months in AC (n=28) and AA (n=40) carriers. More specifically this analysis indicates that for rs9582036 (SEQ ID NO. 2), patients with AA genotype have longer survival than patient with AC genotypes, and patients with AC genotypes have longer survival than patients with CC genotype. A similar association was observed with the progression free survival end-point. After adjustment for baseline prognostic factors (neutrophil counts, CRP and tumor location) the effect was attenuated but not suppressed. No effect was detected in the control group. A test for interaction between rs9582036 (SEQ ID NO. 2) genotype and treatment resulted in a p-value of 0.02. Likewise, the hazard ratio for TG and GG carriers in the rs9554316 (SEQ ID NO. 1) SNP was 2.07 (CI=1.25-3.43; p=0.0047) and 4.69 (CI=1.75-12.95; p=0.0021). Specifically this analysis indicates that for rs9554316 (SEQ ID NO. 1), patients with GG genotype have longer survival than patient with TG genotypes, and patients with TG genotypes have longer survival than patients with TT genotype. Intriguingly, two other SNPs in the VEGFR-1 gene, i.e. rs9513070 (SEQ ID NO. 3) and rs9554320 (SEQ ID NO. 4), also correlated with improved median overall survival. All 4 SNPs were located in the same chromosomal region encompassing exons 25 to 30 of the VEGFR-1 gene and coding for the essential receptor tyrosine-kinase (TK) domain.

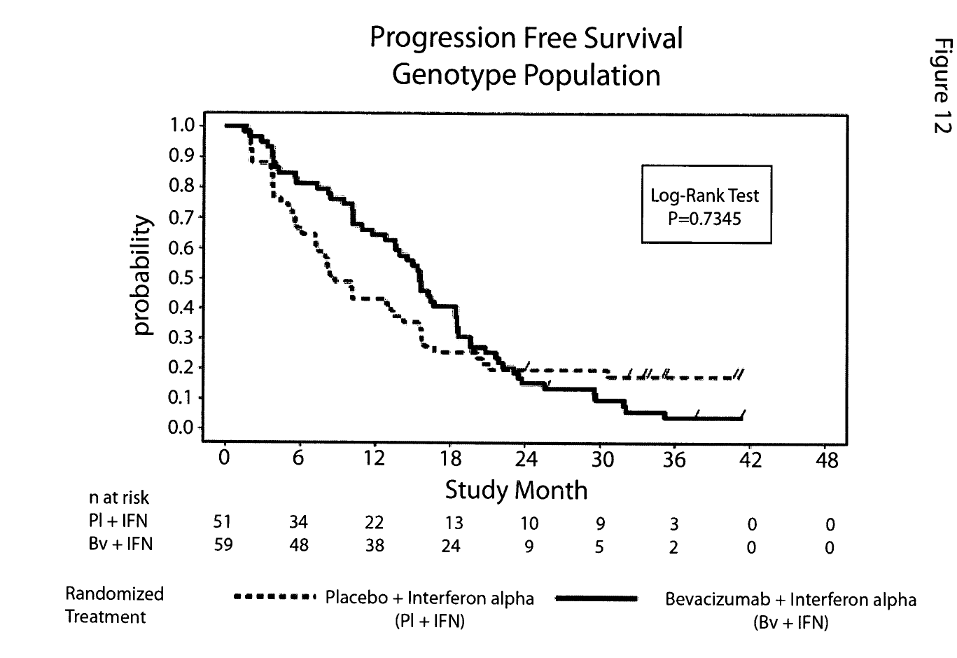

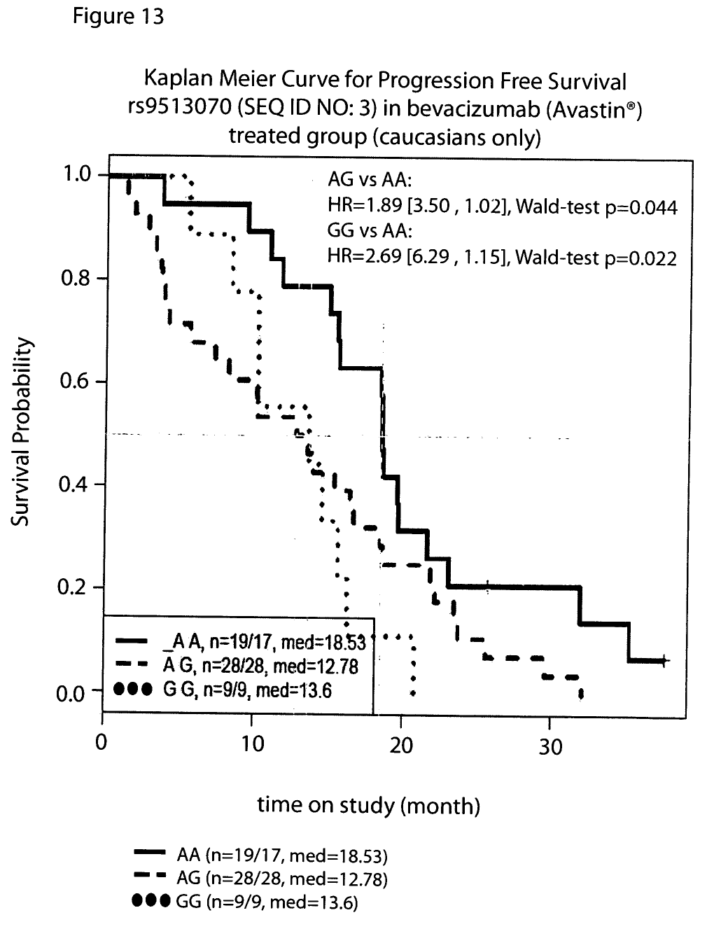

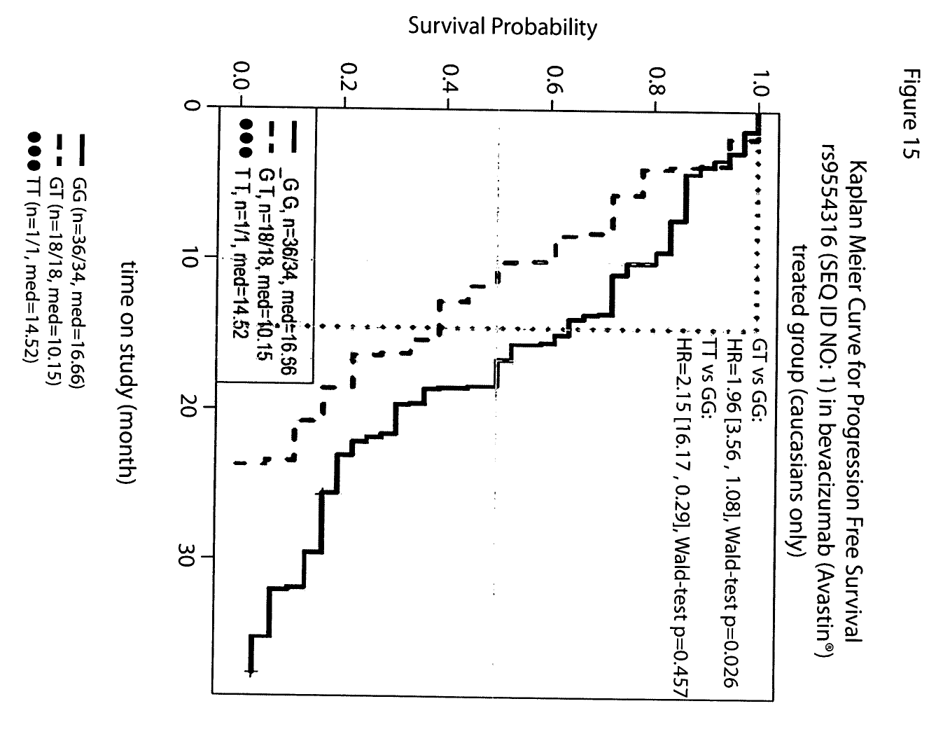

In accordance with the methods of the present invention, two SNPs in the VEGFR-1 gene correlated with progression-free survival in the bevacizumab-treated group in a study treating renal cell cancer with interferon alpha plus bevacizumab or interferon alpha plus placebo. The two SNPs identified are derived for SNPs rs9554316 (SEQ ID NO. 1) and rs9513070 (SEQ ID NO. 3). AA carriers of rs9513070 (SEQ ID NO: 3) showed increased progression free survival in comparison to AG and GG carriers in the bevacizumab-treated group (FIG. 13). GG carriers of rs9554316 (SEQ ID NO:1) showed increased progression free survival in comparison to GT and TT carriers in the bevacizumab-treated group (FIG. 15).

Accordingly, the present invention provides an angiogenesis inhibitor for use in an improved chemotherapy regimen for a patient suffering from a malignant disease or a disease involving physiological and pathological angiogenesis wherein it is determined from a patient sample if the patient has one or more variant alleles of the VEGFR-1 gene and whereby the angiogenesis inhibitor is to be administered to a patient having one or more variant alleles of the VEGFR-1 gene. The angiogenesis inhibitor to be administered may be administered with other chemotherapeutic agents or chemotherapy regimens. The malignant disease or a disease involving physiological and pathological angiogenesis may be pancreatic cancer or renal cell cancer. The angiogenesis inhibitor may be bevacizumab.

The present invention relates to bevacizumab for use in an improved chemotherapy regimen for a patient suffering from pancreatic cancer wherein the it is determined from a patient sample if the patient has one or more variant alleles of the VEGFR-1 gene and whereby bevacizumab is to be administered to a patient having one or more variant alleles of the VEGFR-1 gene. The bevacizumab to be administered may be administered with other chemotherapeutic agents or chemotherapy regimens, such as gemcitabine-erlotinib therapy. The present invention relates to bevacizumab for use in an improved chemotherapy regimen for a patient suffering from renal cell cancer wherein the it is determined from a patient sample if the patient has one or more variant alleles of the VEGFR-1 gene and whereby bevacizumab is to be administered to a patient having one or more variant alleles of the VEGFR-1 gene. The bevacizumab to be administered may be administered with other chemotherapeutic agents or chemotherapy regimens, such as interferon alpha therapy.

The following similar uses may be applied mutatis mutandis.

The present invention provides the use of an angiogenesis inhibitor for improving overall survival of a patient suffering from a malignant disease or a disease involving physiological and pathological angiogenesis comprising the following steps:

(a) determining from a patient sample if the patient suffering from a malignant disease or a disease involving physiological and pathological angiogenesis has one or more variant alleles of the VEGFR-1 gene; and
(b) administering the angiogenesis inhibitor to the patient having one or more variant alleles of the VEGFR-1 gene.

The malignant disease or a disease involving physiological and pathological angiogenesis may be pancreatic cancer. The angiogenesis inhibitor may be bevacizumab.

Accordingly, the present invention relates to the use of an angiogenesis inhibitor for improving overall survival of a patient suffering from a malignant disease or a disease involving physiological and pathological angiogenesis comprising the following steps:

(a) obtaining a sample from a patient suffering from a malignant disease or a disease involving physiological and pathological angiogenesis;
(b) determining if the patient has one or more variant alleles of the VEGFR-1 gene; and
(c) administering the angiogenesis inhibitor to the patient having one or more variant alleles of the VEGFR-1 gene.

The malignant disease or a disease involving physiological and pathological angiogenesis may be pancreatic cancer. The angiogenesis inhibitor may be bevacizumab.

The present invention relates to an angiogenesis inhibitor for use in improving overall survival of a patient suffering from a malignant disease or a disease involving physiological and pathological angiogenesis wherein it is determined from a patient sample if the patient suffering from a malignant disease or a disease involving physiological and pathological angiogenesis has one or more variant alleles of the VEGFR-1 gene and whereby the angiogenesis inhibitor is to be administered to the patient having one or more variant alleles of the VEGFR-1 gene. The malignant disease or a disease involving physiological and pathological angiogenesis may be pancreatic cancer. The angiogenesis inhibitor may be bevacizumab.

The present invention provides the use of an angiogenesis inhibitor for improving progression-free survival of a patient suffering from a malignant disease or a disease involving physiological and pathological angiogenesis comprising the following steps:

(a) determining from a patient sample if the patient suffering from a malignant disease or a disease involving physiological and pathological angiogenesis has one or more variant alleles of the VEGFR-1 gene; and
(b) administering the angiogenesis inhibitor to the patient having one or more variant alleles of the VEGFR-1 gene.

The malignant disease or a disease involving physiological and pathological angiogenesis may be pancreatic cancer or renal cell cancer. The angiogenesis inhibitor may be bevacizumab.

Accordingly, the present invention relates to the use of an angiogenesis inhibitor for improving progression-free survival of a patient suffering from a malignant disease or a disease involving physiological and pathological angiogenesis comprising the following steps:

(a) obtaining a sample from a patient suffering from a malignant disease or a disease involving physiological and pathological angiogenesis;
(b) determining if the patient has one or more variant alleles of the VEGFR-1 gene; and
(c) administering the angiogenesis inhibitor to the patient having one or more variant alleles of the VEGFR-1 gene.

The malignant disease or a disease involving physiological and pathological angiogenesis may be pancreatic cancer or renal cell cancer. The angiogenesis inhibitor may be bevacizumab.

The present invention relates to an angiogenesis inhibitor for use in improving progression-free survival of a patient suffering from a malignant disease or a disease involving physiological and pathological angiogenesis wherein it is determined from a patient sample if the patient suffering from a malignant disease or a disease involving physiological and pathological angiogenesis has one or more variant alleles of the VEGFR-1 gene and whereby the angiogenesis inhibitor is to be administered to said patient having one or more variant alleles of the VEGFR-1 gene. The malignant disease or a disease involving physiological and pathological angiogenesis may be pancreatic cancer or renal cell cancer. The angiogenesis inhibitor may be bevacizumab.

Accordingly, the invention provides the use of an angiogenesis inhibitor for improving overall survival of a patient suffering from a metastatic pancreatic cancer comprising the following steps:

(a) determining from a patient sample if the patient suffering from metastatic pancreatic cancer has one or more variant alleles of the VEGFR-1 gene wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(b) administering the angiogenesis inhibitor to the patient having one or more variant alleles of the VEGFR-1 gene.

The angiogenesis inhibitor may be bevacizumab. The angiogenesis inhibitor may be administered with other chemotherapeutic agents or chemotherapy regimens, such as gemcitabine-erlotinib.

The invention relates to the use of an angiogenesis inhibitor for improving overall survival of a patient suffering from a metastatic pancreatic cancer comprising the following steps:

(a) obtaining a sample from a patient suffering from metastatic pancreatic cancer;
(b) determining from a patient sample if the patient has one or more variant alleles of the VEGFR-1 gene wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(c) administering the angiogenesis inhibitor to the patient having one or more variant alleles of the VEGFR-1 gene.

The angiogenesis inhibitor may be bevacizumab. The angiogenesis inhibitor may be administered with other chemotherapeutic agents or chemotherapy regimens, such as gemcitabine-erlotinib.

The present invention, therefore, relates to an angiogenesis inhibitor for use in improving overall survival of a patient suffering from metastatic pancreatic cancer wherein it is determined from a patient sample if the patient suffering from metastatic pancreatic cancer has one or more variant alleles of the VEGFR-1 gene and whereby the angiogenesis inhibitor it to be administered to the patient having one or more variant alleles of the VEGFR-1 gene. The angiogenesis inhibitor may be bevacizumab. The angiogenesis inhibitor may be administered with other chemotherapeutic agents or chemotherapy regimens, such as gemcitabine-erlotinib therapy.

Accordingly, the invention provides the use of an angiogenesis inhibitor for improving progression-free survival of a patient suffering from a metastatic pancreatic cancer comprising the following steps:

(a) determining from a patient sample if the patient suffering from metastatic pancreatic cancer has one or more variant alleles of the VEGFR-1 gene wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(b) administering the angiogenesis inhibitor to the patient having one or more variant alleles of the VEGFR-1 gene.

The angiogenesis inhibitor may be bevacizumab. The angiogenesis inhibitor may be administered with other chemotherapeutic agents or chemotherapy regimens, such as gemcitabine-erlotinib therapy.

Accordingly, the invention relates to the use of an angiogenesis inhibitor for improving progression-free survival of a patient suffering from a metastatic pancreatic cancer comprising the following steps:

(a) obtaining a sample from a patient suffering from metastatic pancreatic cancer;
(b) determining from a patient sample if the patient has one or more variant alleles of the VEGFR-1 gene wherein the one or more variant alleles are selected from the group consisting of SNPs rs9554316 (SEQ ID NO. 1), rs9582036 (SEQ ID NO. 2), rs9513070 (SEQ ID NO. 3) and rs9554320 (SEQ ID NO.4); and
(c) administering the angiogenesis inhibitor to the patient having one or more variant alleles of the VEGFR-1 gene.

The angiogenesis inhibitor may be bevacizumab. The angiogenesis inhibitor may be administered with other chemotherapeutic agents or chemotherapy regimens, such as gemcitabine-erlotinib therapy.

The present invention, therefore, relates to an angiogenesis inhibitor for use in improving progression-free survival of a patient suffering from metastatic pancreatic cancer wherein it is determined from a patient sample if the patient suffering from metastatic pancreatic cancer has one or more variant alleles of the VEGFR-1 gene and whereby the angiogenesis inhibitor is to be administered to the patient having one or more variant alleles of the VEGFR-1 gene. The angiogenesis inhibitor may be bevacizumab. The angiogenesis inhibitor may be administered with other chemotherapeutic agents or chemotherapy regimens, such as gemcitabine-erlotinib therapy.

Accordingly, the invention provides the use of an angiogenesis inhibitor for improving progression-free survival of a patient suffering from a renal cell cancer comprising the following steps:

(a) determining from a patient sample if the patient suffering from renal cell cancer has one or more variant alleles of the VEGFR-1 gene wherein the one or more variant alleles are selected from the group consisting of rs9554316 (SEQ ID NO. 1) and rs9513070 (SEQ ID NO. 3); and
(b) administering the angiogenesis inhibitor to the patient having one or more variant alleles of the VEGFR-1 gene.

The angiogenesis inhibitor may be bevacizumab. The angiogenesis inhibitor may be administered with other chemotherapeutic agents or chemotherapy regimens, such as interferon alpha therapy.

Accordingly, the invention relates to the use of an angiogenesis inhibitor for improving progression-free survival of a patient suffering from a renal cell cancer comprising the following steps:

(a) obtaining a sample from a patient suffering from renal cell cancer;
(b) determining if the patient has one or more variant alleles of the VEGFR-1 gene wherein the one or more variant alleles are selected from the group consisting of rs9554316 (SEQ ID NO. 1) and rs9513070 (SEQ ID NO. 3); and
(c) administering the angiogenesis inhibitor to the patient having one or more variant alleles of the VEGFR-1 gene.

The angiogenesis inhibitor may be bevacizumab. The angiogenesis inhibitor may be administered with other chemotherapeutic agents or chemotherapy regimens, such as interferon alpha therapy.

The present invention, therefore, relates to an angiogenesis inhibitor for use in improving progression-free survival of a patient suffering from renal cell cancer wherein it is determined from a patient sample if the patient suffering from renal cell cancer has one or more variant alleles of the VEGFR-1 gene and whereby the angiogenesis inhibitor is to be administered to the patient having one or more variant alleles of the VEGFR-1 gene is administered an angiogenesis inhibitor. The angiogenesis inhibitor may be bevacizumab. The angiogenesis inhibitor may be administered with other chemotherapeutic agents or chemotherapy regimens, such as interferon alpha therapy.

In accordance with the methods of the present invention, the herein identified SNPs are predictive of patients suffering from a malignant disease or a disease involving physiological and pathological angiogenesis, in particular metastatic pancreatic cancer or renal cell cancer, that is responsive or sensitive to treatment with an angiogenesis inhibitor, in particular bevacizumab. Accordingly, the present invention relates to a method of predicting the response to or sensitivity to an angiogenesis inhibitor of a patient suffering from a malignant disease or a disease involving physiological and pathological angiogenesis comprising determining from a patient sample if a patient suffering from a malignant disease or a disease involving physiological and pathological angiogenesis has one or more variant alleles of the VEGFR-1 gene. The malignant disease or a disease involving physiological and pathological angiogenesis may be pancreatic cancer or renal cell cancer. The angiogenesis inhibitor may be bevacizumab.

The present invention provides a method of predicting the response to or sensitivity to angiogenesis inhibitor therapy of a patient suffering from metastatic pancreatic cancer comprising determining from a patient sample if a patient suffering from metastatic pancreatic cancer has one or more variant alleles of the VEGFR-1 gene. The one or more variant alleles of the VEGFR-1 gene may be selected from the group consisting of SNPs rs9554316 (SEQ ID NO. 1), rs9582036 (SEQ ID NO. 2), rs9513070 (SEQ ID NO. 3) and rs9554320 (SEQ ID NO.4), whereby a G at position 51 of rs9554316 (SEQ ID NO. 1), an A at position 51 of rs9582036 (SEQ ID NO. 2), an A at position 51 of rs9513070 (SEQ ID NO. 3) and/or a C at position 51 of rs9554320 (SEQ ID NO.4) is indicative of a patient response or sensitive to angiogenesis inhibitor therapy. The angiogenesis inhibitor may be bevacizumab. The invention, therefore, relates to the use of specific primers and/or probes for the preparation of a composition for predicting the response to or sensitivity to angiogenesis inhibitor therapy of a patient suffering from metastatic pancreatic cancer wherein the primers and/or probes are capable of detecting at least one of the variant alleles selected from the group consisting of SNPs rs9554316 (SEQ ID NO. 1), rs9582036 (SEQ ID NO. 2), rs9513070 (SEQ ID NO. 3) and rs9554320 (SEQ ID NO.4), whereby a G at position 51 of rs9554316 (SEQ ID NO. 1), an A at position 51 of SNPs rs9582036 (SEQ ID NO. 2), an A at position 51 of rs9513070 (SEQ ID NO. 3) and/or a C at position 51 of rs9554320 (SEQ ID NO.4) is indicative of a patient response or sensitive to angiogenesis inhibitor therapy. The angiogenesis inhibitor may be bevacizumab.

The present invention also provides a method of predicting the response to or sensitivity to angiogenesis inhibitor therapy of a patient suffering from renal cell cancer comprising determining from a patient sample if the patient suffering from renal cell cancer has one or more variant alleles of the VEGFR-1 gene from a patient sample wherein the one or more variant alleles are selected from the group consisting of SNPs rs9554316 (SEQ ID NO. 1) and rs9513070 (SEQ ID NO. 3) whereby a G at position 51 of rs9554316 (SEQ ID NO. 1) and/or an A at position 51 of rs9513070 (SEQ ID NO. 3) is indicative of a patient response or sensitive to angiogenesis inhibitor therapy. The angiogenesis inhibitor may be bevacizumab. The invention, therefore, relates to the use of specific primers and/or probes for the preparation of a composition for predicting the response to or sensitivity to bevacizumab therapy of a patient suffering from renal cell cancer wherein the primers and/or probes are capable of detecting at least one of the variant alleles are selected from the group consisting of at least one of the variant alleles selected from the group consisting of SNPs rs9554316 (SEQ ID NO. 1) and rs9513070 (SEQ ID NO. 3) whereby a G at position 51 of rs9554316 (SEQ ID NO. 1) and/or an A at position 51 of rs9513070 (SEQ ID NO. 3) is indicative of a patient response or sensitive to angiogenesis inhibitor therapy. The angiogenesis inhibitor may be bevacizumab.

The sample to be used in the methods provided herein is a biological sample and may be a blood and/or tissue sample. Preferably, the sample is a blood sample and more preferably a peripheral blood sample. Even more preferably, the sample is a DNA sample. The DNA sample may be germline DNA or somatic DNA. Preferably, the DNA is germline DNA.

The sample is preferably obtained from a patient suffering from a malignant disease or a disease involving physiological and pathological angiogenesis. Examples of a malignant disease include, but are not limited to, pancreatic cancer, renal cell cancer, lung cancer, breast cancer, colorectal cancer, prostate cancer, lymphoma, bone cancer, ovarian cancer, melanoma, prostate haematological malignancy and/or metastatic forms of the above identified cancers. Preferably the sample is obtained from a patient suffering from pancreatic cancer, renal cell cancer, lung cancer, breast cancer and/or colorectal cancer. Even more preferably the sample is obtained from a patient suffering from metastatic pancreatic cancer, renal cell cancer, metastatic colorectal cancer, metastatic breast cancer and/or non-squamous non-small cell lung cancer. Most preferably, the patient is suffering from metastatic pancreatic cancer or renal cell cancer. Examples of disease involving physiological and pathological angiogenesis include, but are not limited to, high grade glioma, glioblastoma, M. Rendu-Osler, von-Hippel-Lindau diseases, hemangiomas, psoriasis, Kaposi's sarcoma, ocular neovascularisation, rheumatoid arthritis, endometriosis, atherosclerosis, myochardial ischemia, peripheral ischemia, cerebral ischemia and wound healing.

The terms “angiogenesis inhibitor” in the context of the present invention refers to all agents that alter angiogenesis (e.g. the process of forming blood vessels) and includes agents that inhibit the angiogenesis, including, but not limited to, tumor angiogenesis. In this context, inhibition can refer to blocking the formation of blood vessels and halting or slowing down the growth of blood vessels. Examples of angiogenesis inhibitors include bevacizumab (also known as Avastin®), pegaptanib, sunitinib, sorafenib and vatalanib. A preferred angiogenesis inhibitors is bevacizumab (Avastin®), which is a recombinant humanized monoclonal IgG1 antibody that binds to and inhibits the biological activity of human VEGFA in in vitro and in vivo assay system. The term “bevacizumab” encompass all corresponding anti-VEGF antibodies that fulfill the requirements necessary for obtaining a marketing authorization as an identical or biosimilar product in a country or territory selected from the group of countries consisting of the USA, Europe and Japan.

In the context of the present invention, “administration” or “administering” means the administration of a pharmaceutical composition, such as an angiogenesis inhibitor, to the patient. For example, 2.5 mg/kg of body weight to 15 mg/kg of body weight bevacizumab (Avastin®) can be administered every week, every 2 weeks or every 3 weeks, depending on the type of cancer being treated. Preferred dosages include 5 mg/kg, 7.5 mg/kg, 10 mg/kg and 15 mg/kg. Even more preferred dosages are 5 mg/kg every 2 weeks, 10 mg/kg every 2 weeks and 15 mg/kg every 3 weeks. With respect to bevacizumab (Avastin®) for the treatment of metastatic pancreatic cancer, dosages include 5 mg/kg or 10 mg/kg of body weight given once every 2 weeks, or 7.5 mg/kg or 15 mg/kg of body weight given once every 3 weeks. For the treatment of renal cell cancer, dosages of bevacizumab (Avastin®) include 10 mg/kg of body weight given once every 2 weeks.

Dosages of with bevacizumab (Avastin®) for treatments of specific cancers, according to the EMEA, are as follows (for details see http://www.emea.europa.eu/humandocs/PDFs/EPAR/avastin/emea-combined-h582en.pdf). For metastatic carcinoma of the colon or rectum (mCRC) preferred dosages are 5 mg/kg or 10 mg/kg of body weight given once every 2 weeks or 7.5 mg/kg or 15 mg/kg of body weight given once every 3 weeks, for metastatic breast cancer (mBC) preferred dosages are 10 mg/kg of body weight given once every 2 weeks or 15 mg/kg of body weight given once every 3 weeks as an intravenous infusion, and for non-small cell lung cancer (NSCLC) preferred dosages are 7.5 mg/kg or 15 mg/kg of body weight given once every 3 weeks as an intravenous infusion. Clinical benefit in NSCLC patients has been demonstrated with both 7.5 mg/kg and 15 mg/kg doses. For details refer to section 5.1 Pharmacodynamic Properties, Non-small cell lung cancer (NSCLC). For advanced and/or metastatic Renal Cell Cancer (mRCC) preferred dosages are 10 mg/kg of body weight given once every 2 weeks as an intravenous infusion (in addition to platinum-based chemotherapy for up to 6 cycles of treatment followed by bevacizumab (Avastin®) as a single agent until disease progression). For gliablastoma a preferred dosage is 10 mg/kg every 2 weeks.

In the context of the present invention, the angiogenesis inhibitor may be administered in addition to or as a co-therapy or a co-treatment with one or more chemotherapeutic agents administered as part of standard chemotherapy regimen as known in the art. Examples of agents included in such standard chemotherapy regimens include 5-fluorouracil, leucovorin, irinotecan, gemcitabine, erlotinib, capecitabine, taxanes, such as docetaxel and paclitaxel, interferon alpha, vinorelbine, and platinum-based chemotherapeutic agents, such as paclitaxel, carboplatin, cisplatin and oxaliplatin. Examples of co-treatments for metastatic pancreatic cancer include gemcitabine-erlotinib plus bevacizumab at a dosage of 5 mg/kg or 10 mg/kg of body weight given once every two weeks or 7.5 mg/kg or 15 mg/kg of body weight given once every three weeks. Examples of co-treatments for renal cell cancer include interferon alpha plus bevacizumab at a dosage of or 10 mg/kg of body weight given once every two weeks. Further, a patient may be co-treated with a combination of irinotecan, 5-fluorouracil, leucovorin, also referred to as IFL, as, for example, a bolus-IFL, with a combination of oxaliplatin, leucovorin, and 5-fluorouracil, also referred to a FOLFOX4 regimen, or with a combination of capecitabine and oxaliplatin, also referred to as XELOX. Accordingly, in a further embodiment of the invention, the patient suffering from a malignant disease or a disease involving physiological and pathological angiogenesis is being treated with one or more chemotherapeutic agents such as 5-fluorouracil, leucovorin, irinotecan, gemcitabine-erlotinib, capecitabine and/or platinum-based chemotherapeutic agents, such as paclitaxel, carboplatin and oxaliplatin. Examples of co-therapy or co-treatment include 5 mg/kg bevacizumab (Avastin®) every two week with bolus-IFL or 10 mg/kg bevacizumab (Avastin®) every 2 weeks with FOLFOX4 for metastatic colorectal cancer, 15 mg/kg bevacizumab (Avastin®) every 3 weeks with caboplatis/paclitaxel for non-squamous non-small cell lung cancer, and 10 mg/kg bevacizumab (Avastin®) every 2 weeks with paclitaxel for metastatic breast cancer. Further, the angiogenesis inhibitor to be administered may be administered as a co-therapy or a co-treatment with radiotherapy.

As used herein “chemotherapeutic agent” or “chemotherapy regimen” includes any active agent that can provide an anticancer therapeutic effect and may be a chemical agent or a biological agent, in particular, that are capable of interfering with cancer or tumor cells. Preferred active agents are those that act as anti-neoplastic (chemotoxic or chemostatic) agents which inhibit or prevent the development, maturation or proliferation of malignant cells. Nonlimiting examples of chemotherapeutic agents include alkylating agents such as nitrogen mustards (e.g., mechlorethamine, cyclophosphamide, ifosfamide, melphalan and chlorambucil), nitrosoureas (e.g., carmustine (BCNU), lomustine (CCNU), and semustine (methyl-CCNU)), ethylenimines/methylmelamines (e.g., thriethylenemelamine (TEM), triethylene, thiophosphoramide (thiotepa), hexamethylmelamine (HMM, altretamine)), alkyl sulfonates (e.g., busulfan), and triazines (e.g., dacarbazine (DTIC)); antimetabolites such as folic acid analogs (e.g., methotrexate, trimetrexate), pyrimidine analogs (e.g., 5-fluorouracil, capecitabine, fluorodeoxyuridine, gemcitabine, cytosine arabinoside (AraC, cytarabine), 5-azacytidine, 2,2′-difluorodeoxycytidine), and purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs developed from natural products (e.g., paclitaxel, vinca alkaloids (e.g., vinblastine (VLB), vincristine, and vinorelbine), docetaxel, estramustine, and estramustine phosphate), epipodophylotoxins (.e.g., etoposide, teniposide), antibiotics (.e.g, actimomycin D, daunomycin (rubidomycin), daunorubicon, doxorubicin, epirubicin, mitoxantrone, idarubicin, bleomycins, plicamycin (mithramycin), mitomycinC, actinomycin), enzymes (e.g., L-asparaginase), and biological response modifiers (e.g., interferon-alpha, IL-2, G-CSF, GM-CSF); miscellaneous agents including platinum coordination complexes (e.g., cisplatin, carboplatin, oxaliplatin), anthracenediones (e.g., mitoxantrone), substituted urea (i.e., hydroxyurea), methylhydrazine derivatives (e.g., N-methylhydrazine (MIH), procarbazine), adrenocortical suppressants (e.g., mitotane (o,p′-DDD), aminoglutethimide); hormones and antagonists including adrenocorticosteroid antagonists (.e.g, prednisone and equivalents, dexamethasone, aminoglutethimide), progestins (e.g., hydroxyprogesterone caproate, medroxyprogesterone acetate, megestrol acetate), estrogens (e.g., diethylstilbestrol, ethinyl estradiol and equivalents thereof); antiestrogens (e.g., tamoxifen), androgens (e.g., testosterone propionate, fluoxymesterone and equivalents thereof), antiandrogens (e.g., flutamide, gonadotropin-releasing hormone analogs, leuprolide), non-steroidal antiandrogens (e.g., flutamide), epidermal growth factor inhibitors (e.g., erlotinib, lapatinib, gefitinib) antibodies (e.g., trastuzumab), irinotecan and other agents such as leucovorin. For the treatment of metastatic pancreatic cancer, chemotherapeutic agents for administration with bevacizumab include gemcitabine and erlotinib and combinations thereof (see also the examples herein provided). For the treatment of renal cell cancer, chemotherapeutic agents for administration with bevacizumab include interferon alpha (see also the examples herein provided).

The terms “responder for an angiogenesis inhibitor” means in the context of the present invention that a subject/patient suffering from a malignant disease or a disease involving physiological and pathological angiogenesis shows a response to a treatment with the angiogenesis inhibitor. A skilled person will readily be in a position to determine whether a person treated with the angiogenesis inhibitor shows a response. For example, a response to an angiogenesis inhibitor may be reflected by decreased suffering from a malignant disease or a disease involving physiological and pathological angiogenesis, such as a diminished and/or halted growth of a tumor and/or a reduction of the size of a tumor, the prevention of the formation of metastases or a reduction of number or size of metastases.

The terms “patient sensitive to an angiogenesis inhibitor” in the context of the present invention refers to a patient which shows in some way a positive reaction when treated with an angiogenesis inhibitors. The reaction of the patient may be less pronounced when compared to a responder as described hereinabove. For example, the patient may experience less suffering from a malignant disease, such as cancer, though no reduction in tumor growth may be measured. The reaction of the patient to the angiogenesis inhibitor may also be only of a transient nature, i.e. growth of (a) tumor and/or (a) metastasis(es) may only be temporarily reduced or halted.

The terms “a patient suffering from” in the context of the present invention refers to a patient showing clinical signs in respect to a certain malignant disease, such as cancer, a disease involving physiological and pathological angiogenesis and/or tumorous disease.

The terms “for improving overall survival” in the context of the present invention refer to the average survival of the patient within a patient group. As the skilled person will appreciate, a patient's overall survival is improved or enhanced, if the patient belongs to a subgroup of patients that has a statistically significant longer mean survival time as compared to another subgroup of patients.

The terms “for improving progression-free survival” in the context of the present invention refer, with respect to a patient within a patient group, to the average length of time during and after treatment in which a patient's disease does not get worse. As the skilled person will appreciate, a patient's progression-free survival is improved or enhanced, if the patient belongs to a subgroup of patients that has a significantly longer mean length of time during which the disease does not get worse compared to another subgroup of patients.

The terms “oligonucleotide” and “polynucleotide” are used interchangeably and in the context of the present invention refer to a molecule comprised of two or more deoxyribonucleotides or ribonucleotides, preferably more than three. Its exact size will depend on many factors, which in turn depend on the ultimate function or use of the oligonucleotide. An oligonucleotide can be derived synthetically or by cloning. Chimeras of deoxyribonucleotides and ribonucleotides may also be in the scope of the present invention.

Sequence amplification is a method for generating large amounts of a target sequence. In general, one or more amplification primers are annealed to a nucleic acid sequence. Using appropriate enzymes, sequences found adjacent to or in between the primers are amplified.

Amplification primer is an oligonucleotide that is capable of annealing to a target sequence and servings as an initiation point for DNA synthesis when placed under conditions in which the synthesis of primer extension product that is complementary to a nucleic acid is initiated. Primers according to the present invention can be designed as commonly known in the art based on the sequences of the SNPs provided herein. Primers are preferably 10 to 45 nucleotides long and more preferably 15 to 30 nucleotides long.

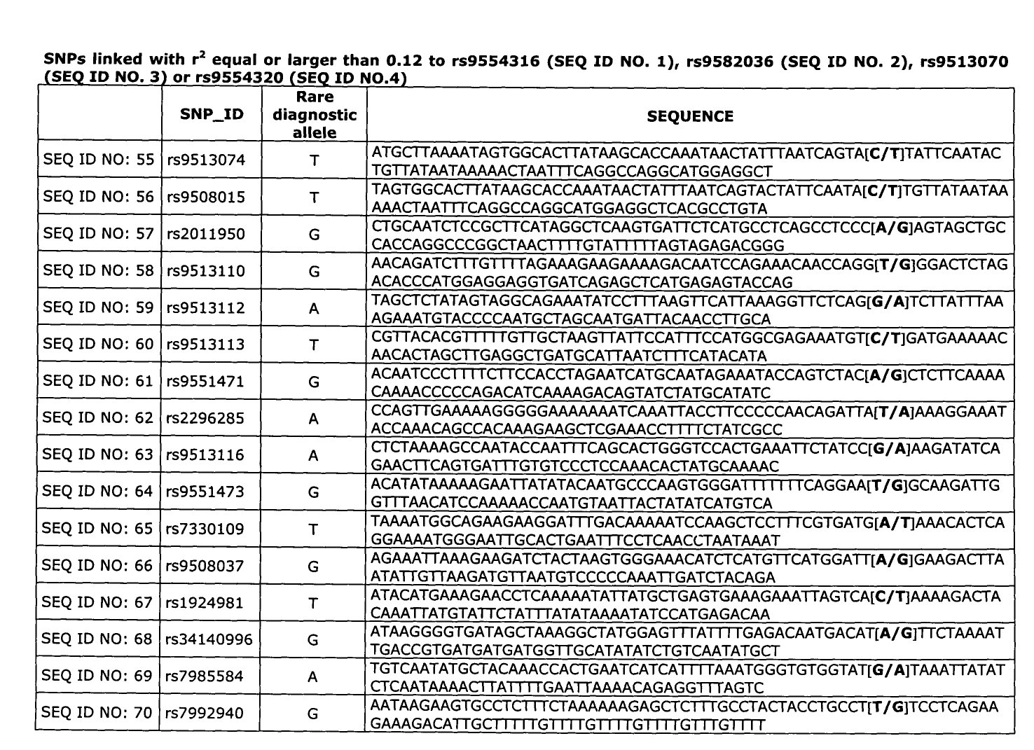

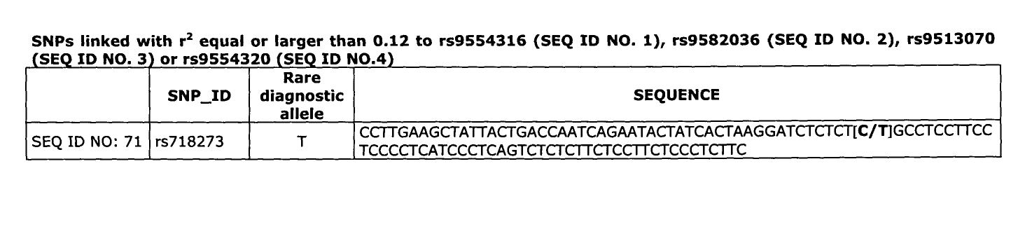

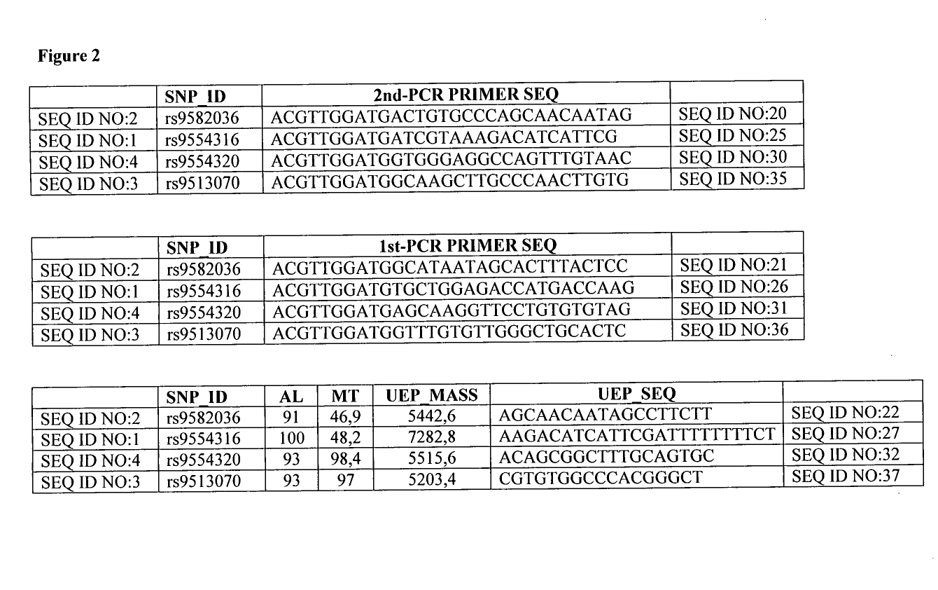

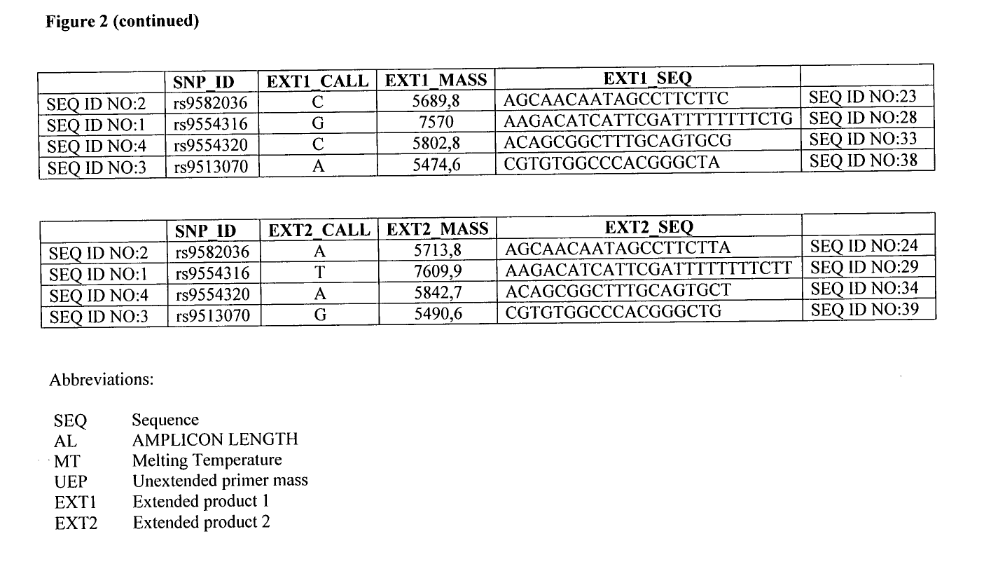

According to the invention herein described, a primer designed to bind upstream of a SNP is used in combination with a second primer designed to bind downstream of the same SNP. Examples of nucleic acid primers derived for SNPs rs9582036 (SEQ ID NO. 2), rs9554316 (SEQ ID NO. 1), rs9513070 (SEQ ID NO. 3) and rs9554320 (SEQ ID NO. 4) are shown in FIG. 2. More specifically, for rs9554316 (SEQ ID NO. 1), ACGTTGGATGATCGTAAAGACATCATTCG (SEQ ID NO. 25) and ACGTTGGATGTGCTGGAGACCATGACCAAG (SEQ ID NO. 26) can be used. For SNPs rs9582036 (SEQ ID NO. 2), ACGTTGGATGACTGTGCCCAGCAACAATAG (SEQ ID NO. 20) and ACGTTGGATGGCATAATAGCACTTTACTCC (SEQ ID NO. 21) can be used. For rs9513070 (SEQ ID NO. 3), ACGTTGGATGGCAAGCTTGCCCAACTTGTG (SEQ ID NO. 35) and ACGTTGGATGGTTTGTGTTGGGCTGCACTC (SEQ ID NO. 36) can be used. For rs9554320 (SEQ ID NO.4), ACGTTGGATGGTGGGAGGCCAGTTTGTAAC (SEQ ID NO. 30) and ACGTTGGATGAGCAAGGTTCCTGTGTGTAG (SEQ ID NO. 31) can be used.

As will be understood by the person of ordinary skill, a multitude of probes can be designed for each SNP identified herein. For example, probes that are extendable primers, which may be extended in a termination extension reaction, are shown in FIG. 2 with there extension products. For rs9554316 (SEQ ID NO. 1), AAGACATCATTCGATTTTTTTTCT (SEQ ID NO. 27) was used. For SNPs rs9582036 (SEQ ID NO. 2), AGCAACAATAGCCTTCTT (SEQ ID NO. 22) was used. For rs9513070 (SEQ ID NO. 3), CGTGTGGCCCACGGGCT (SEQ ID NO. 37) was used. For rs9554320 (SEQ ID NO.4), ACAGCGGCTTTGCAGTGC (SEQ ID NO. 32) was used. Probes that are extendable primers are preferably 15 to 30 nucleotides long and more preferably 15 to 25 nucleotides long.

The present invention provides oligonucleotides or polynucleotides useful for determining the presence of at least one or more variant alleles of the VEGF-R1 gene. The oligonucleotides or polynucleotides may comprise primers and/or probes. With respect to primers, the primers may be selected from the group consisting of SEQ ID NO. 20, SEQ ID NO. 25, SEQ ID NO. 30, SEQ ID NO. 35, SEQ ID NO. 21, SEQ ID NO. 26, SEQ ID NO. 31 and SEQ ID NO. 36. With respect to probes, that the probes may be selected form the group consisting of SEQ ID NO. 22, SEQ ID NO. 27, SEQ ID NO. 32 and SEQ ID NO. 37.

A skilled person will also have the ability to use probes, such as hybridization probes, that can be labelled by standard labelling techniques such as with a radiolabel, enzyme label, fluorescent label, biotin-avidin label, chemiluminescence, and the like. After hybridization, the probes can be visualized using known techniques.

The present invention also relates to a diagnostic composition or kit comprising any of the mentioned oligonucleotides and optionally suitable means for detection.

The kit of the invention may advantageously be used for carrying out a method of the invention and could be, inter alia, employed in a variety of applications, e.g., in the diagnostic field or as a research tool. The parts of the kit of the invention can be packages individually in vials or in combination in containers or multicontainer units. Manufacture of the kit follows preferably standard procedures which are known to the person skilled in the art. The kit or diagnostic compositions may be used for detection of the one or more variant alleles of the VEGFR-1 gene in accordance with the herein-described methods of the invention, employing, for example, amplification techniques as described herein.

Accordingly, in a further embodiment of the present invention provides a kit useful for carrying out the methods herein described, comprising oligonucleotides or polynucleotides capable of determining the presence of at least one or more variant alleles of the VEGF-R1 gene. The oligonucleotides or polynucleotides may comprise primers and/or probes. With respect to primers, the primers may be selected from the group consisting of SEQ ID NO. 20, SEQ ID NO. 25, SEQ ID NO. 30, SEQ ID NO. 35, SEQ ID NO. 21, SEQ ID NO. 26, SEQ ID NO. 31 and SEQ ID NO. 36. With respect to probes, the probes may be selected form the group consisting of SEQ ID NO. 22, SEQ ID NO. 27, SEQ ID NO. 32 and SEQ ID NO. 37.

The present invention is further described by reference to the following non-limited figures and examples.

FIG. 1: Sequence of SNPs. The diagnostic allele for each SNP is identified. FIG. 1A are SNPs genotyped in AVITA study and associated with bevacizumab outcome. FIG. 1B are linked SNPs belonging to the rs9554316 (SEQ ID NO. 1) and rs9582036 (SEQ ID NO. 2) haplotype block (within the locus). FIG. 1C are linked SNPs 5′ up or downstream of the haplotype block (outside the locus) and in LD (D′>0.8) with rs9554316 (SEQ ID NO. 1) and rs9582036 (SEQ ID NO. 2). FIG. 1D are linked SNPs with r² equal or larger than 0.12 to rs9554316 (SEQ ID NO. 1), rs9582036 (SEQ ID NO. 2), rs9513070 (SEQ ID NO. 3) or rs9554320 (SEQ ID NO.4).

FIG. 2: Primers for SNPs rs9554316 (SEQ ID NO. 1), rs9582036 (SEQ ID NO. 2), rs9513070 (SEQ ID NO. 3) and rs9554320 (SEQ ID NO. 4). The unextended primers (probes), extended primers (probes) and their masses are also provided.

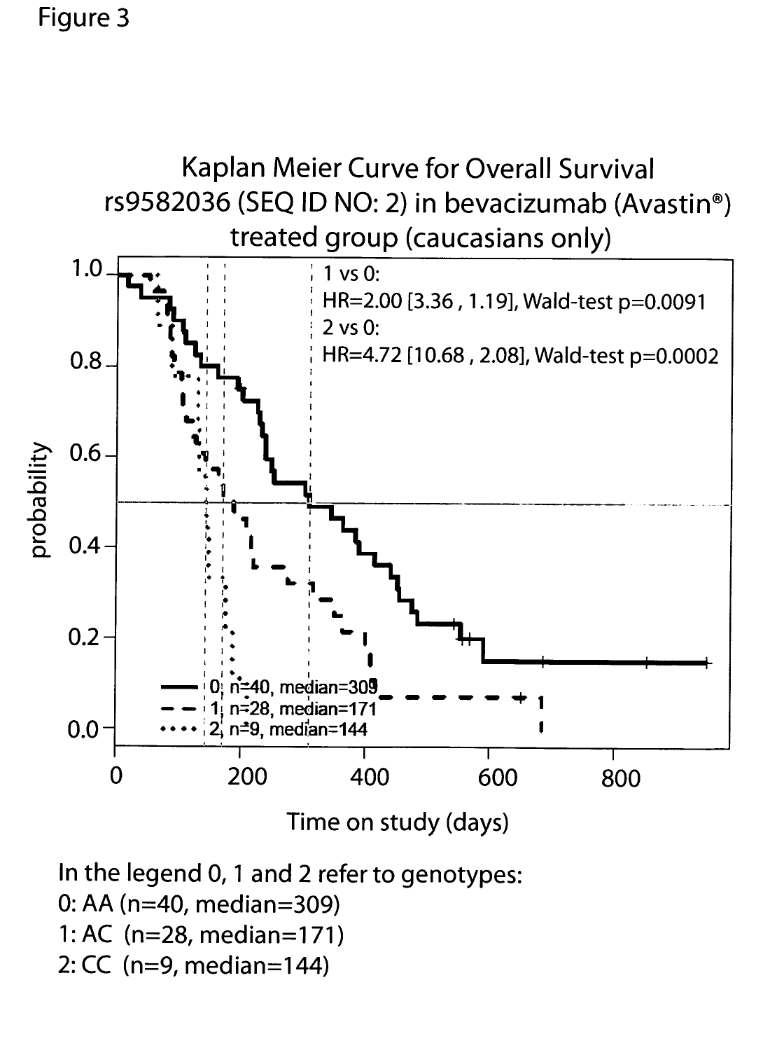

FIG. 3: Kaplan Meier curve: Overall Survival, probability vs. time on study (days), by rs9582036 (SEQ ID NO. 2) genotype in bevacizumab treated sub-group of AVITA trial (patients with metastatic pancreatic cancer treated with bevacizumab plus gemcitabine-erlotinib (GE) therapy). In the legend 0, 1 and 2 refer to the genotypes: 0 is AA (n=40, median=309), 1 is AC (n=28, median=171) and 2 is CC (n=9, median=144). 1 vs 0 (HR=2.00 [3.36, 1.19], Wald-test p=0.0091) and 2 vs 0: (HR=4.72 [10.68, 2.08], Wald-test p=0.0002). In the figure, the solid line is for genotype AA, the dashed line is for genotype AC, and the dotted line is for genotype CC.

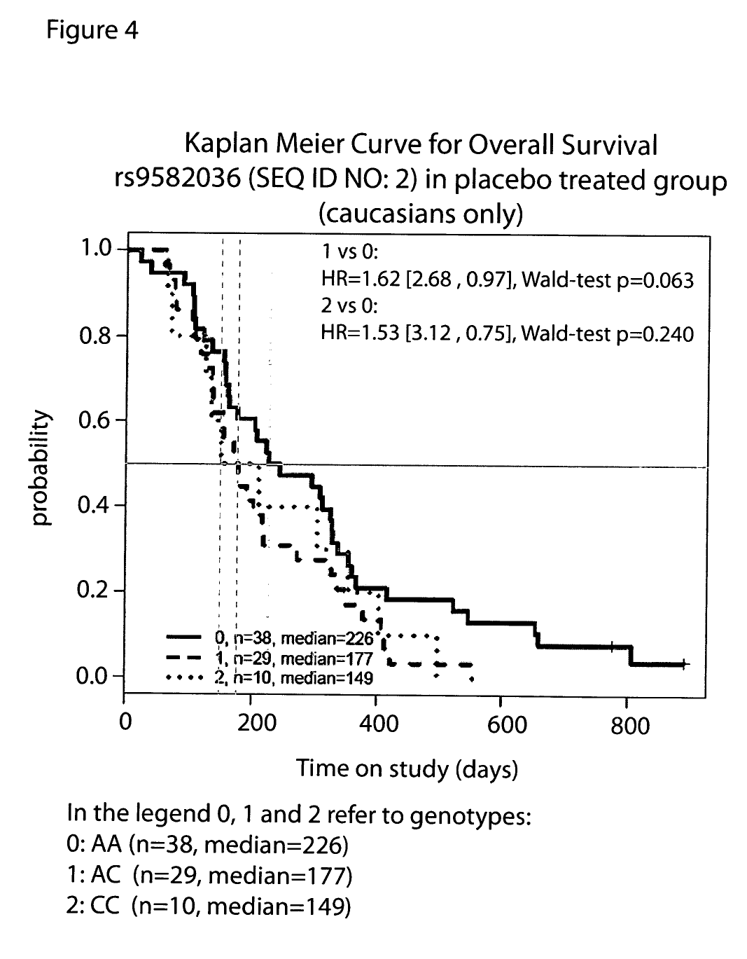

FIG. 4: Kaplan Meier curve: Overall Survival, probability vs. time on study (days), by rs9582036 (SEQ ID NO. 2) genotype in placebo treated sub-group of AVITA trial (patients with metastatic pancreatic cancer treated with placebo plus gemcitabine-erlotinib (GE) therapy). In the legend 0, 1 and 2 refer to the genotypes: 0 is AA (n=38, median=226), 1 is AC (n=29, median=177) and 2 is CC (n=10, median=149). 1 vs 0 (HR=1.62 [2.68, 0.97], Wald-test p=0.063) and 2 vs 0: (HR=1.53 [3.12, 0.75], Wald-test p=0.240). In the figure, the solid line is for genotype AA, the dashed line is for genotype AC, and the dotted line is for genotype CC.

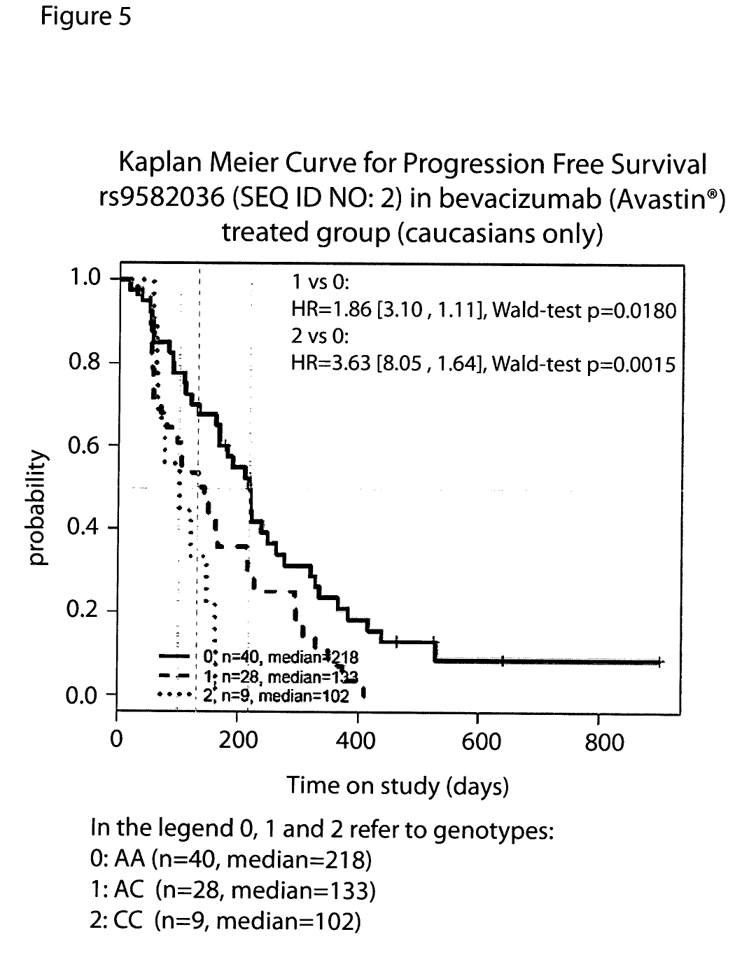

FIG. 5: Kaplan Meier curve: Progression Free Survival, probability vs. time on study (days), by rs9582036 (SEQ ID NO. 2) genotype in bevacizumab treated sub-group of AVITA trial (patients with metastatic pancreatic cancer treated with bevacizumab plus gemcitabine-erlotinib (GE) therapy). In the legend 0, 1 and 2 refer to the genotypes: 0 is AA (n=40, median=218), 1 is AC (n=28, median=133) and 2 is CC (n=9, median=102). 1 vs 0 (HR=1.86 [3.10, 1.11], Wald-test p=0.0180) and 2 vs 0: (HR=3.63 [8.05, 1.64], Wald-test p=0.0015). In the figure, the solid line is for genotype AA, the dashed line is for genotype AC, and the dotted line is for genotype CC.

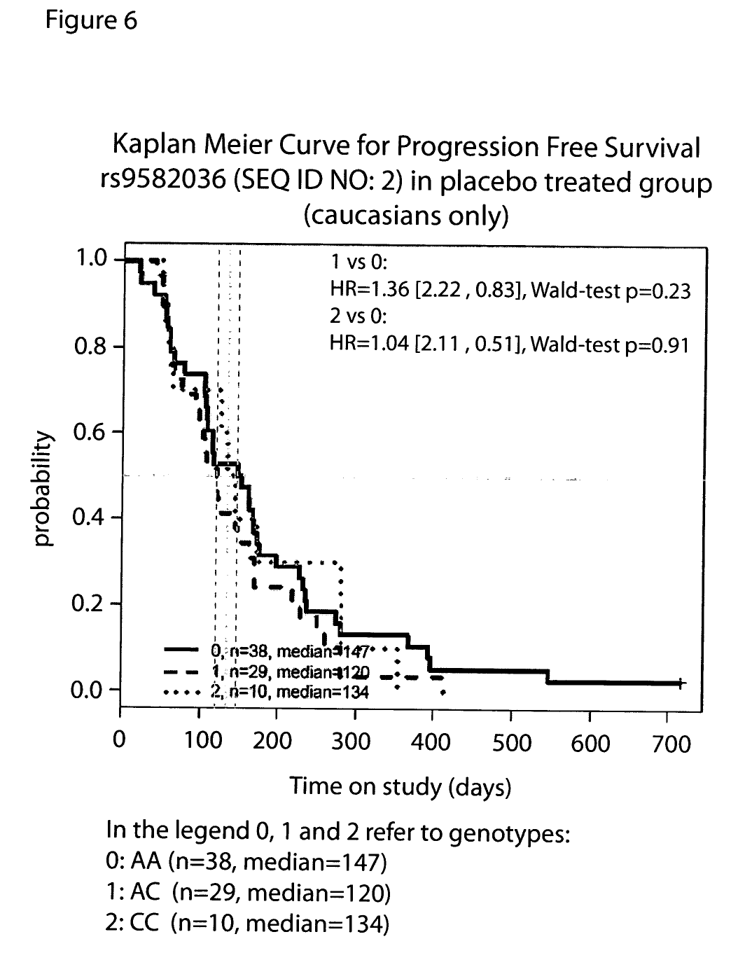

FIG. 6: Kaplan Meier curve: Progression Free Survival, probability vs. time on study (days), by rs9582036 (SEQ ID NO. 2) genotype in placebo treated sub-group of AVITA trial (patients with metastatic pancreatic cancer treated with placebo plus gemcitabine-erlotinib (GE) therapy). In the legend 0, 1 and 2 refer to the genotypes: 0 is AA (n=38, median=147), 1 is AC (n=29, median=120) and 2 is CC (n=10, median=134). 1 vs 0 (HR=1.36 [2.22, 0.83], Wald-test p=0.23) and 2 vs 0: (HR=1.04 [2.11, 0.51], Wald-test p=0.91). In the figure, the solid line is for genotype AA, the dashed line is for genotype AC, and the dotted line is for genotype CC.

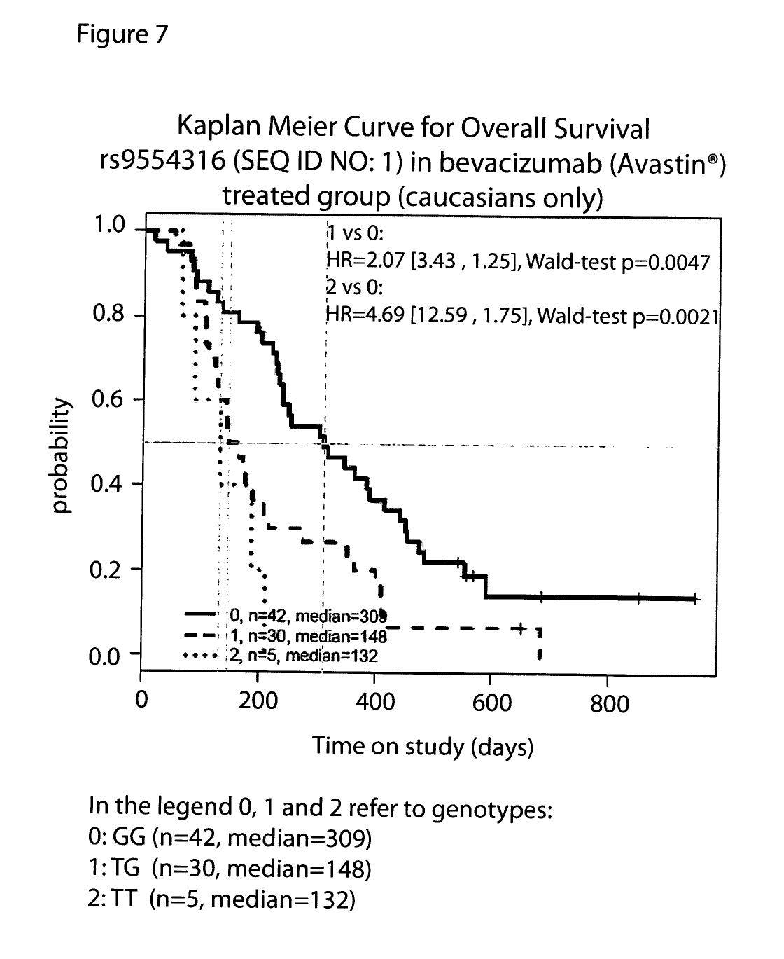

FIG. 7: Kaplan Meier curve: Overall Survival, probability vs. time on study (days), by rs9554316 (SEQ ID NO. 1) genotype in bevacizumab treated sub-group of AVITA trial (patients with metastatic pancreatic cancer treated with bevacizumab plus gemcitabine-erlotinib (GE) therapy). In the legend 0, 1 and 2 refer to the genotypes: 0 is GG (n=42, median=309), 1 is TG (n=30, median=148) and 2 is TT (n=5, median=132). 1 vs 0 (HR=2.07 [3.43, 1.25], Wald-test p=0.0047) and 2 vs 0: (HR=4.69 [12.59, 1.75], Wald-test p=0.0021). In the figure, the solid line is for genotype GG, the dashed line is for genotype TG, and the dotted line is for genotype TT.

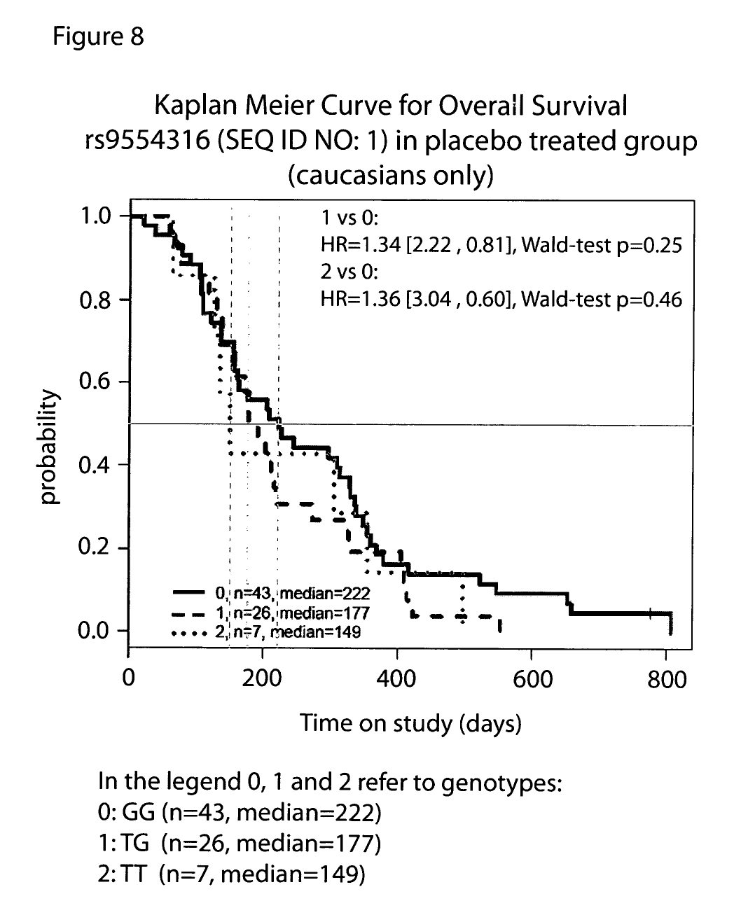

FIG. 8: Kaplan Meier curve: Overall Survival, probability vs. time on study (days), by rs9554316 (SEQ ID NO. 1) genotype in placebo treated sub-group of AVITA trial (patients with metastatic pancreatic cancer treated with placebo plus gemcitabine-erlotinib (GE) therapy). In the legend 0, 1 and 2 refer to the genotypes: 0 is GG (n=43, median=222), 1 is TG (n=26, median=177) and 2 is TT (n=7, median=149). 1 vs 0 (HR=1.34 [2.22, 0.81], Wald-test p=0.25) and 2 vs 0: (HR=1.36 [3.04, 0.60], Wald-test p=0.46). In the figure, the solid line is for genotype GG, the dashed line is for genotype TG, and the dotted line is for genotype TT.

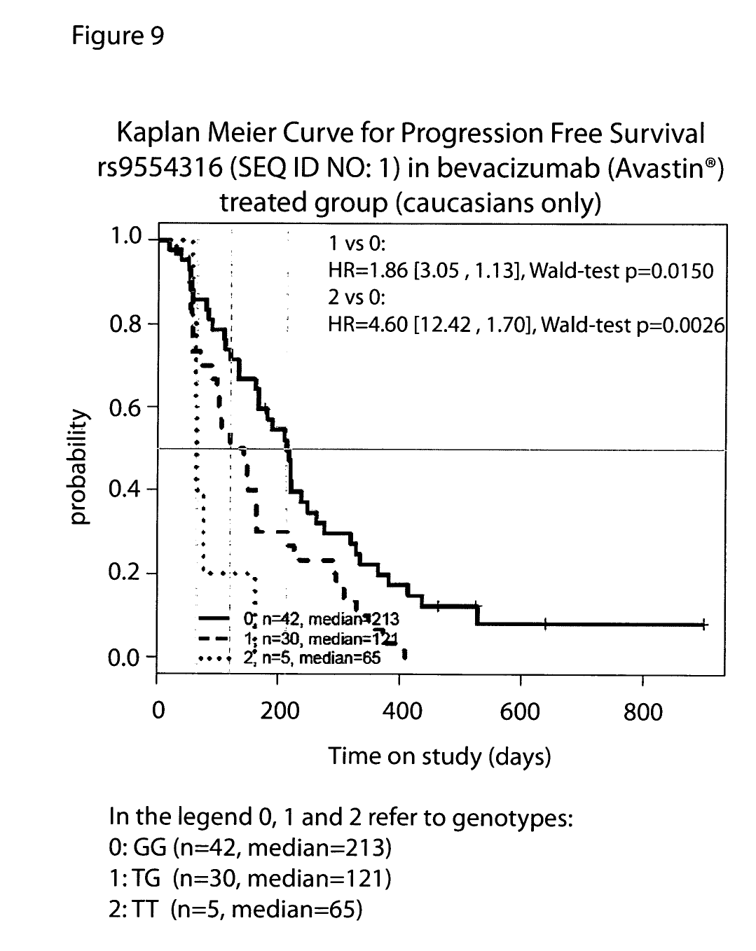

FIG. 9: Kaplan Meier curve: Progression Free Survival, probability vs. time on study (days), by rs9554316 (SEQ ID NO. 1) genotype in bevacizumab treated sub-group of AVITA trial (patients with metastatic pancreatic cancer treated with bevacizumab plus gemcitabine-erlotinib (GE) therapy). In the legend 0, 1 and 2 refer to the genotypes: 0 is GG (n=42, median=213), 1 is TG (n=30, median-121) and 2 is TT (n=5, median=65). 1 vs 0 (HR=1.86 [3.05, 1.13], Wald-test p=0.0150) and 2 vs 0: (HR=4.60 [12.42, 1.70], Wald-test p=0.0026). In the figure, the solid line is for genotype GG, the dashed line is for genotype TG, and the dotted line is for genotype TT.

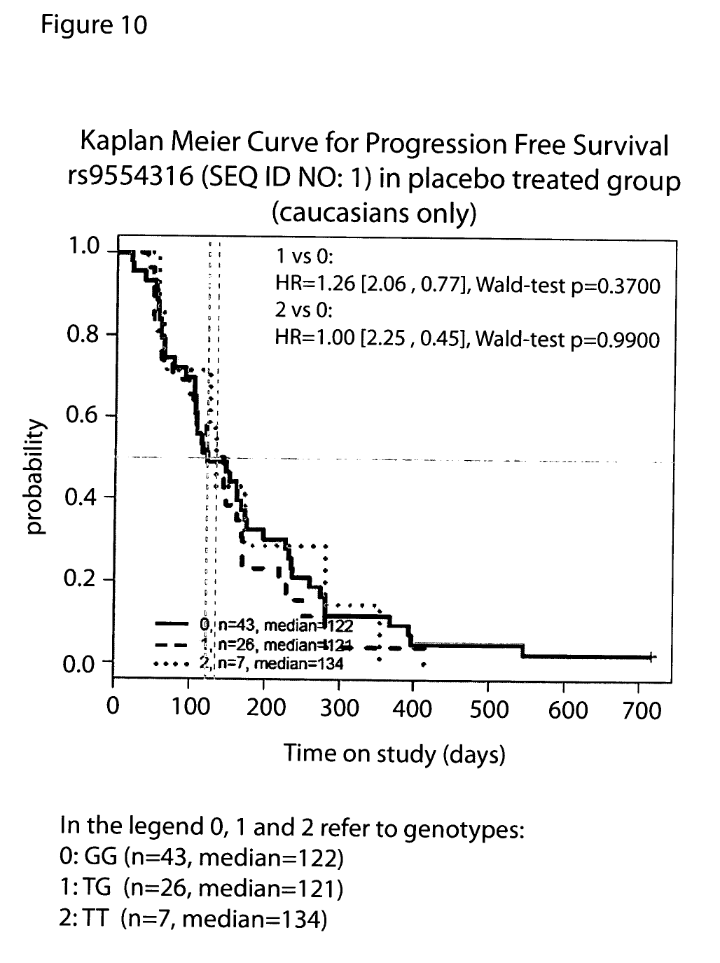

FIG. 10: Kaplan Meier curve: Progression Free Survival, probability vs. time on study (days), by rs9554316 (SEQ ID NO. 1) genotype in placebo treated sub-group of AVITA trial (patients with metastatic pancreatic cancer treated with placebo plus gemcitabine-erlotinib (GE) therapy). In the legend 0, 1 and 2 refer to the genotypes: 0 is GG (n=43, median=122), 1 is TG (n=26, median=121) and 2 is TT (n=7, median=134). 1 vs 0 (HR=1.26 [2.06, 0.77], Wald-test p=0.3700) and 2 vs 0: (HR=1.00 [2.25, 0.45], Wald-test p=0.9900). In the figure, the solid line is for genotype GG, the dashed line is for genotype TG, and the dotted line is for genotype TT.

FIG. 11: Kaplan Meier curve: Progression Free Survival, probability vs. study month, for overall population of AVOREN trial (patients with renal cell cancer treated with bevacizumab plus interferon alpha (IFN) therapy or placebo plus interferon alpha (IFN) therapy). In the figure, the dashed line is for patients treated with placebo plus interferon alpha and the solid line is for patients treated with bevacizumab plus interferon alpha.

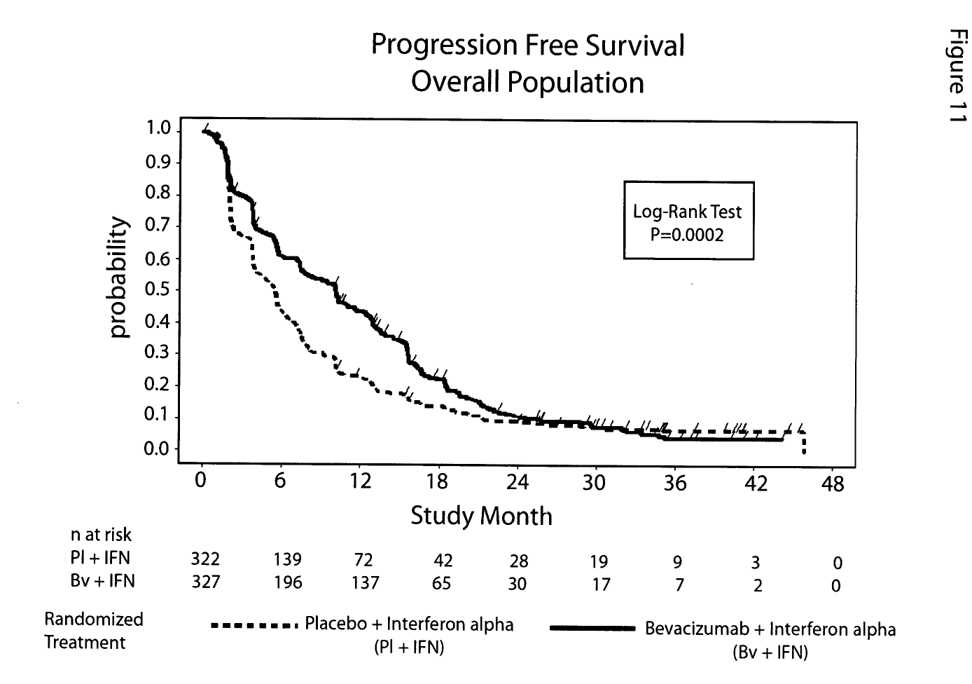

FIG. 12: Kaplan Meier curve: Progression Free Survival, probability vs. study month, for genotype population of AVOREN trial (patients with renal cell cancer treated with bevacizumab plus interferon alpha (IFN) therapy or placebo plus interferon alpha (IFN) therapy). In the figure, the dashed line is for patients treated with placebo plus interferon alpha and the solid line is for patients treated with bevacizumab plus interferon alpha.

FIG. 13: Kaplan Meier curve: Progression Free Survival, probability vs. time on study (month), by rs9513070 (SEQ ID NO. 3) genotype in bevacizumab treated sub-group of AVOREN trial (patients with renal cell cancer treated with bevacizumab plus interferon alpha (IFN) therapy). The genotypes are AA (n=19/17, med=18.53), AG (n=28/28, med=12.78) and GG (n=9/9, med=13.6). AG vs AA (HR=1.89 [3.50, 1.02], Wald-test p=0.044) and GG vs AA: (HR=2.69 [6.29, 1.15], Wald-test p=0.022). In the figure, the solid line is for genotype AA, the dashed line is for genotype AG, and the dotted line is for genotype GG.

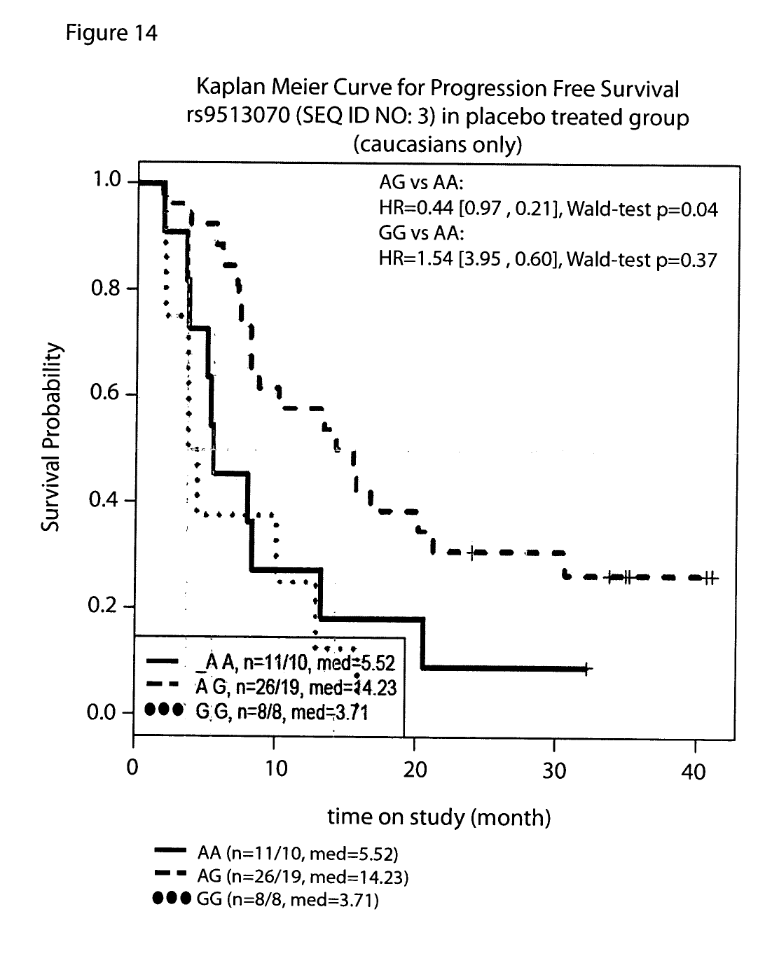

FIG. 14: Kaplan Meier curve: Progression Free Survival, probability vs. time on study (month), by rs9513070 (SEQ ID NO. 3) genotype in placebo treated sub-group of AVOREN trial (patients with renal cell cancer treated with placebo plus interferon alpha (IFN) therapy). The genotypes are AA (n=11/10, med=5.52), AG (n=26/19, med=14.23) and GG (n=8/8, med=3.71). AG vs AA (HR=0.44 [0.97, 0.21], Wald-test p=0.04) and GG vs AA: (HR=1.54 [3.95, 0.60], Wald-test p=0.37). In the figure, the solid line is for genotype AA, the dashed line is for genotype AG, and the dotted line is for genotype GG.

FIG. 15: Kaplan Meier curve: Progression Free Survival, probability vs. time on study (month), by rs9554316 (SEQ ID NO. 1) genotype in bevacizumab treated sub-group of AVOREN trial (patients with renal cell cancer treated with bevacizumab plus interferon alpha (IFN) therapy). The genotypes are GG (n=36/34, med=16.66), GT (n=18/18, med=10.15) and TT (n=1/1, med=14.52). GT vs GG (HR=1.96 [3.56, 1.08], Wald-test p=0.026) and TT vs GG: (HR=2.15 [16.17, 0.29], Wald-test p=0.457). In the figure, the solid line is for genotype GG, the dashed line is for genotype GT, and the dotted line is for genotype TT.

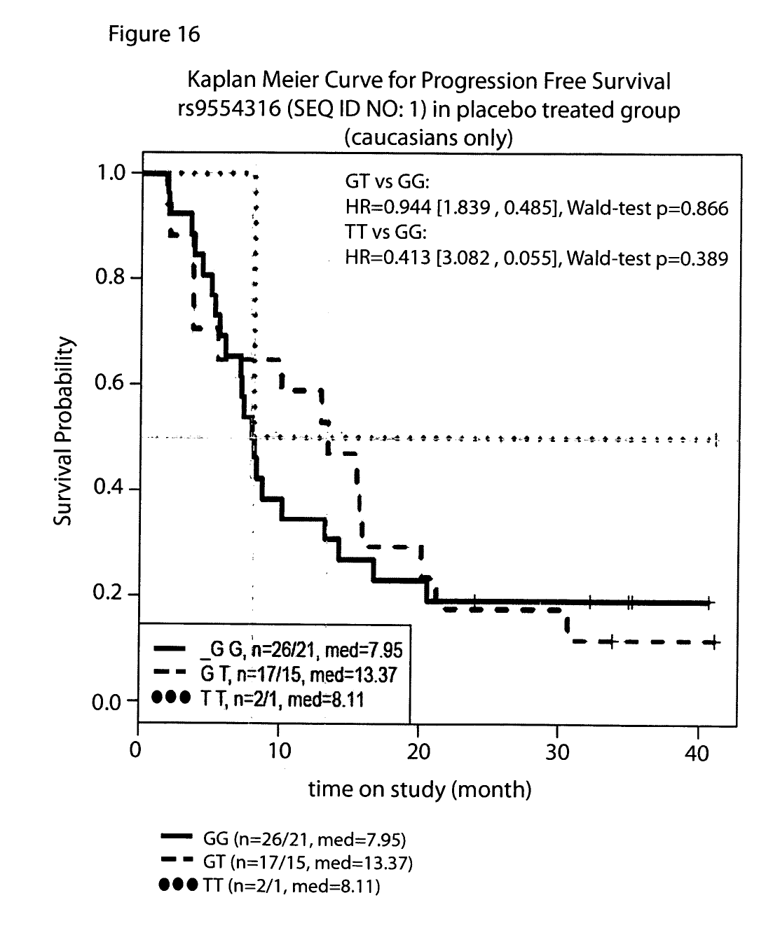

FIG. 16: Kaplan Meier curve: Progression Free Survival, probability vs. time on study (month), by rs9554316 (SEQ ID NO. 1) genotype in placebo treated sub-group of AVOREN trial (patients with renal cell cancer treated with placebo plus interferon alpha (IFN) therapy). The genotypes are GG (n=26/21, med=7.95), GT (n=17/15, med=13.37) and TT (n=2/1, med=8.11). GT vs GG (HR=0.944 [1.839, 0.485], Wald-test p=0.866) and TT vs GG: (HR=0.413 [3.082, 0.055], Wald-test p=0.389). In the figure, the solid line is for genotype GG, the dashed line is for genotype GT, and the dotted line is for genotype TT.

As documented in the appended illustrative examples, the means and methods provided herein relate to analysis and evaluation of biological samples from Caucasians.

### EXAMPLE 1

Genetic determination can influence sensitivity of the endothelium to VEGF. In accordance with this and in context of this invention, we explored genetic variability in underlying signaling pathways in order to discover predictive patterns for anti angiogenic treatment. In this analysis, the correlation of genetic variability in the VEGF signaling pathway with clinical outcome of patients with metastatic pancreatic cancer treated with gemcitabine-erlotinib (GE) plus bevacizumab or placebo in a phase III trial (AVITA) was evaluated.

Germline DNA was available from 154 out of 607 patients, of which 77 received GE plus bevacizumab and 77 GE alone (median overall survival=7.5 and 6.8 months); see AVITA study (BO17706) accessible at http://www.roche-trials.com/patient/trials/trial11.html. Common single nucleotide polymorphisms (SNPs) located in the hypoxia-inducible factor-1α and -2α, VEGF, its receptors (VEGFR-1 and -2) and other relevant genes were selected using a SNP tagging approach (f≧0.1 and r²≦0.8). 157 SNPs were successfully genotyped using MALDI-TOF mass spectrometry. Risk and survival estimates were calculated using Cox regression analyses.

Four SNPs in the VEGFR-1 gene correlated with overall survival and progression-free survival in the bevacizumab-treated group. The most significant SNP, rs9582036, had p-value<=3e-4 in an allelic risk effect model. Relative to AA carriers, the hazard ratio was 2.0 (CI=1.2-3.4; p=0.009) and 4.7 (CI=2.1-10.7; p=0.0002) for AC and CC carriers, respectively. Median overall survival was increased from 4.8 months in CC (n=9) carriers to 6.0 and 10.3 months in AC (n=28) and AA (n=40) carriers. A similar association was observed with the PFS end-point. After adjustment for baseline prognostic factors (neutrophil counts, CRP and tumor location) the effect was attenuated but not suppressed. No effect was detected in the control group. A test for interaction between rs9582036 (SEQ ID NO. 2) genotype and treatment resulted in a p value of 0.02. All 4 SNPs were located in the same chromosomal region encompassing exons 25 to 30 of the VEGFR-1 gene and coding for the essential receptor tyrosine-kinase (TK) domain. It was, furthermore, surprisingly found that no association with the previously reported VEGF-2578C/A polymorphism could be detected in this data set.

These subgroup data provide for a genetic locus in the VEGFR-1 gene, in particular, in the TK domain of VEGFR-1 that correlates with clinical outcome in cancer patients, for example in pancreatic cancer patients treated with an angiogenesis inhibitor, like bevacizumab. Accordingly, predictive values using the herein described parameters can be obtained. These parameters, i.e. the herein described variations of the VEGFR-1 gene can be used for the assessment of the beneficial effect of anti-angiogenic treatment in subjects suffering from cancer. Accordingly, the invention as provided herein relates to the provision of novel and inventive biomarkers for predictive as well as prognostic assessments.

### Patients and Methods

### Samples

In the AVITA trial, there were 607 eligible patients. The trial protocol was approved by the institutional review board at each site and was conducted in accordance with the Declaration of Helsinki, current US Food and Drug Administration Good Clinical Practices, and local ethical and legal requirements. Frozen whole-blood samples were available from 160 out of 607 patients (26.4%), 154 of these had a disease progression event and for 150 patients the disease was fatal. Of these 160 patients, 6 were of Asian ethnicity, while all remaining 154 patients were of Caucasian origin. As patients of Asian origin are genetically distinct from Caucasian patients and SNP frequencies may differ between both ethnic groups, these 6 patients were omitted from further analysis. The remaining 154 DNA specimens were provided to the investigators in a de-identified fashion. All corresponding patients provided separate written informed consent for genetic biomarker testing.

### Assessments

Patients were assessed as described previously (Van Cutsem et al., J. Clinc. Oncol. 27, 2231-7 (2009)). Briefly, tumor assessments were made according to Response Evaluation Criteria in Solid Tumors guidelines at baseline, weeks 8, 16, 24, 32, 40, and every 12 weeks thereafter until disease progression. Patients were followed for survival and subsequent disease treatment until death, loss to follow-up, or trial termination. Assessments of Karnofsky performance status (KPS) and body weight were collected weekly. Adverse events were recorded at every visit using the National Cancer Institute Common Terminology Criteria for Adverse Events version 3.0 (NCICTCAE v3). Blood samples for genetic biomarker analyses were collected before trial treatment began.

### Single Nucleotide Polymorphism Selection

The following genes involved in the VEGF signalling cascade were selected: the VEGF ligand, the VEGF homologues (placental growth factor or PlGF, VEGF-B and -C, as well as VEGF-D or FlGF), the VEGF receptor-2 (KDR or VEGFR-2) and VEGF receptor-1 (FLT1 or VEGFR-1). Genomic sequences 5 kb upstream of the translation start site up to 3′ poly-A adenylation site of each gene, were used to select SNPs from the HapMap database (HapMap Data Rel 24/phaseII Nov08, on NCBI B36 assembly, dbSNP b126). Tagging SNPs were selected using the Tagger (Pe'er, I., et al., Nat. Genet. 38, 663-7 (2006)) as provided in the HAPLOVIEW software package (Barrett, J. C., et al., Bioinformatics. 21, 263-5 (2005)). Only SNPs occurring commonly, i.e., with a minor allele frequency f≧0.1 and a minimum r² threshold ≧0.8 were considered. In total, 167 tagging SNPs were selected following these criteria. Additionally, 11 SNPs located in exonic sequences and inducing non-synonymous amino-acid changes at a frequency f≧0.1 were selected from the dbSNP database, as well as 4 SNPs in VEGF (rs699947, rs833061, rs2010963 and rs3025039), 1 SNP in VEGFR-1 (rsTP53_R-1) and 1 SNP in VEGFR-2 (rs2071559), which previously have been reported to affect function or expression of these genes.

The chromosomal location and position of each SNP within the VEGFR-1 gene for SNPs rs9582036 (SEQ ID NO. 2), rs9554316 (SEQ ID NO. 1), rs9513070 (SEQ ID NO. 3) and rs9554320 (SEQ ID NO.4) is shown, together with the allelic variation and the reference allele.

| | Position | Position to | | Reference | Genotyping | Frequency |
|---|---|---|---|---|---|---|
| Variation ID | (bp) | Transcript | Alleles | Allele | Assay | (N = 154) |
| rs9554320 | 27784927 | Intron 25 | A/C | A | 6 | 0.43 |
| (SEQ ID NO. 4) | | | | | | |
| rs9582036 | 27783408 | Intron 27 | C/A | C | 3 | 0.7 |
| (SEQ ID NO. 2) | | | | | | |
| rs9554316 | 27779335 | Intron 28 | T/G | T | 7 | 0.25 |
| (SEQ ID NO. 1) | | | | | | |
| rs9513070 | 27777839 | Intron 29 | G/A | A | 5 | 0.60 |
| (SEQ ID NO. 3) | | | | | | |

### Genotyping

Peripheral blood was sampled in K2EDTA plastic Vacutainer tubes. After centrifugation, germ-line DNA was extracted from the precipitated leucocyte cell fraction according to standard procedures. Genotyping for the selected SNPs was carried out at the Vesalius Research Center using the Sequenom® iPLEX platform, as described previously by the manufacturer. All the SNPs that failed to provide robust genotypes in a first genotyping round were re-designed using a different set of PCR primers and tested again. In case of repetitive failure in the second design these SNPs were considered as failed. Of the 184 selected SNPs, 27 SNPs (14.7%) failed and 157 (85.3%) were successfully genotyped. The overall genotype success rate was 98.5%. At the individual sample level, all samples were genotyped with a high success rate (i.e., <5% of the samples had a success rate <95%, whereas >70% of the samples had a success rate >99%). No samples were excluded from the analysis and no significant deviations from Hardy-Weinberg were observed.

The amplification primers designed for SNPs rs9582036 (SEQ ID NO. 2), rs9554316 (SEQ ID NO. 1), rs9513070 (SEQ ID NO. 3) and rs9554320 (SEQ ID NO.4) are shown in FIG. 2. For rs9554316 (SEQ ID NO. 1), ACGTTGGATGATCGTAAAGACATCATTCG (SEQ ID NO. 25) and ACGTTGGATGTGCTGGAGACCATGACCAAG (SEQ ID NO. 26) were used. For SNP rs9582036 (SEQ ID NO. 2), ACGTTGGATGACTGTGCCCAGCAACAATAG (SEQ ID NO. 20) and ACGTTGGATGGCATAATAGCACTTTACTCC (SEQ ID NO. 21) were used. For rs9513070 (SEQ ID NO. 3), ACGTTGGATGGCAAGCTTGCCCAACTTGTG (SEQ ID NO. 35) and ACGTTGGATGGTTTGTGTTGGGCTGCACTC (SEQ ID NO. 36) were used. For rs9554320 (SEQ ID NO.4), ACGTTGGATGGTGGGAGGCCAGTTTGTAAC (SEQ ID NO. 30) and ACGTTGGATGAGCAAGGTTCCTGTGTGTAG (SEQ ID NO. 31) were used.

The unextended primers (probes) designed for SNPs rs9582036 (SEQ ID NO. 2), rs9554316 (SEQ ID NO. 1), rs9513070 (SEQ ID NO. 3) and rs9554320 (SEQ ID NO.4) are also shown in FIG. 2. For rs9554316 (SEQ ID NO. 1), AAGACATCATTCGATTTTTTTTCT (SEQ ID NO. 28) was used. For SNPs rs9582036 (SEQ ID NO. 2), AGCAACAATAGCCTTCTT (SEQ ID NO. 22) was used. For rs9513070 (SEQ ID NO. 3), CGTGTGGCCCACGGGCT (SEQ ID NO. 37) was used. For rs9554320 (SEQ ID NO.4), ACAGCGGCTTTGCAGTGC (SEQ ID NO. 32) was used.

### Statistics

When the allelic model was used, patients homozygous with the minor allele were coded as 2, heterozygous patients were coded as 1, and patients homozygous for the major allele were coded as 0. An independent, separate model was modelled for each SNP.

The Cox proportional hazard regression model was used to evaluate the effect of different genotypes on Overall Survival and Progression Free Survival.

In the Cox model, the time from entry in the study until death or censoring was used as the Overall Survival end-point. Time from entry in the study until disease progression or death was used and the Progression Survival end-point.

For overall survival, the null hypothesis stated that the genotype has no effect on overall survival. The likelihood ratio test was used to calculate a P value corresponding to this hypothesis for each tested SNP.

For progression free survival, the null hypothesis stated that the genotype has no effect on risk of progression or survival. The likelihood ratio test was used to calculate a P value corresponding to this hypothesis for each tested SNP.

The primary analysis were conducted in the sub-group of patients in the bevacizumab arm of the trial. Additional analyses were performed in the sub-group of patients in the placebo arm of the trial, and treatment by genotype interaction was evaluated in model including both.

In the primary analysis, only the genotypes were included as predictors in the model. In order to further characterize and confirm the primary results, some complimentary analyses were conducted with adjustment for ancillary clinical prognostic factors. This was achieved by running additional regression models taking these prognostic factor as covariates in addition to the effect of genotype.

To account for multiple testing, p-value was considered significant if below 0.0005 (5.e-4).

Survival curves and median survival time were calculated using standard Kaplan-Meier estimates.

### Clinical Characteristics of Genetic Patient Population

Since germ-line DNA was obtained from only 26.4% of the study participants (160 out of 607 patients), it was first assessed whether this subgroup exhibited similar disease characteristics as the full patient cohort. All baseline demographics characteristics of the genetics sub-population were similar to the overall study population. There were 77 patients in the placebo arm and 77 patients in the bevacizumab arm. Overall, the subgroup available for genetic analysis behaved very similar, exhibiting a comparable age and gender distribution, smoking status, KPS, visual analogue scale for pain, progression free survival and overall survival. The median overall survival in the sub-group was 7.2 and 6.1 months in the bevacizumab and placebo arm, median progression free survival was 4.6 and 3.6 months respectively.

Baseline demographics were balanced between treatment arms.

| | First trial medication | | | | | |
|---|---|---|---|---|---|---|
| | Bevacizumab | | Placebo | | All | |
| | N | % | N | % | N | % |
| Sex | | | | | | |
| FEMALE | 26 | 37.14 | 25 | 37.31 | 51 | 37.23 |
| MALE | 44 | 62.86 | 42 | 62.69 | 86 | 62.77 |
| Age Category | | | | | | |
| (years) | | | | | | |
| <65 | 41 | 58.57 | 44 | 65.67 | 85 | 62.04 |
| >=65 | 29 | 41.43 | 23 | 34.33 | 52 | 37.96 |
| KPS (%) | | | | | | |
| Category at | | | | | | |
| Baseline | | | | | | |
| <80% | 8 | 11.43 | 6 | 8.96 | 14 | 10.22 |
| >=80% | 62 | 88.57 | 61 | 91.04 | 123 | 89.78 |
| VAS Category | | | | | | |
| at Baseline | | | | | | |
| <20 | 47 | 67.14 | 50 | 74.63 | 97 | 70.80 |
| >=20 | 23 | 32.86 | 17 | 25.37 | 40 | 29.20 |

KPS: karnofsky performance status
VAS: Visual Analogue Scale of Pain at Baseline

SNPs in VEGFR-1 correlated with overall survival in the treatment arm

### Overall Survival Analysis

Analysis in the Sub-Group of Patients Treated with Bevacizumab

Overall Survival by Genotype - No covariates - Subset: Bev Arm

| | snp | gene | HR | CI_95 | pvalue | star | adj. pvalue | N/Nevts |
|---|---|---|---|---|---|---|---|---|
| 152 | rs9582036 | VEGFR1 | 2.113 | (1.45, 3.07) | 1.4e−04 | *** | 2.2e−02 | 77/68 |
| 150 | rs9554316 | VEGFR1 | 2.119 | (1.42, 3.17) | 4.2e−04 | *** | 6.6e−02 | 77/68 |
| 80 | rs3025035 | VEGF | 0.327 | (0.14, 0.77) | 1.7e−03 | ** | 2.6e−01 | 74/65 |
| 146 | rs9513070 | VEGFR1 | 1.67 | (1.14, 2.44) | 8.1e−03 | ** | 1.0e+00 | 74/65 |
| 151 | rs9554320 | VEGFR1 | 1.543 | (1.11, 2.14) | 9.7e−03 | ** | 1.0e+00 | 75/67 |

Analysis in the Sub-Group of Patients Treated with Placebo

Overall Survival by Genotype - No
covariates - Subset: Placebo Arm

| | snp | gene | HR | CI_95 | pvalue | star |
|---|---|---|---|---|---|---|
| 151 | rs9554320 | VEGFR1 | 1.377 | (1.02, 1.85) | 3.6e−02 | * |
| 149 | rs9554311 | VEGFR1 | 1.541 | (1.01, 2.34) | 4.3e−02 | * |
| 134 | rs7982639 | VEGFR1 | 1.554 | (1, 2.42) | 5.5e−02 | |
| 128 | rs7673274 | VEGFR2 | 0.721 | (0.51, 1.02) | 5.8e−02 | |
| 29 | rs1485766 | VEGF-C | 0.707 | (0.49, 1.02) | 6.0e−02 | |

No genotypes were detected to be associated with overall survival in the placebo sub-group.

### Analysis of Treatment by Genotype Interaction

Overall Survival by Genotype - No covariates - Interaction Test - Subset: NULL

| | snp | HR_SNP | HR_treat | HR_inter | pval_inter | N/Nevts | star | gene |
|---|---|---|---|---|---|---|---|---|
| 150 | rs9554316 | 1.189 | 0.587 | 1.814 | 2.3e−02 | 153/143 | * | VEGFR1 |
| 152 | rs9582036 | 1.302 | 0.635 | 1.632 | 4.1e−02 | 154/143 | * | VEGFR1 |
| 19 | rs12505758 | 0.73 | 0.69 | 1.845 | 9.1e−02 | 154/143 | | VEGFR2 |
| 93 | rs3936415 | 1.301 | 1.016 | 0.668 | 1.3e−01 | 154/143 | | VEGFR1 |

A Cox regression analysis was performed for each of the SNPs to assess an effect on overall survival in the treatment arm. To correct for multiple testing, the significance level was set at p<0.0005. Two SNPs predicted a favourable median overall survival (p=0.00014 and p=0.00042 for rs9582036 (SEQ ID NO. 2) and rs9554316 (SEQ ID NO. 1), respectively). Both SNPs were located in VEGFR-1 and demonstrated a superior overall survival with an additive risk effect: relative to rs9582036 (SEQ ID NO. 2) AA carriers, the hazard ratio (HR) was 2.0 (CI=1.2-3.4; p=0.009) and 4.7 (CI=2.1-10.7; p=0.0002) for AC and CC carriers, respectively. Median overall survival was increased from 4.8 months in CC carriers to 6.0 and 10.3 months in AC and AA carriers. Likewise, the hazard ratio for TG and GG carriers in the rs9554316 (SEQ ID NO. 1) SNP was 2.07 (CI=1.25-3.43; p=0.0047) and 4.69 (CI=1.75-12.95; p=0.0021). Intriguingly, two other SNPs in the VEGFR-1 gene, i.e. rs9513070 (SEQ ID NO. 3) and rs9554320 (SEQ ID NO. 4), also correlated with improved median overall survival. These 2 SNPs had very low p-values (p=0.0081 and p=0.0097, respectively), but did not reach the multiplicity adjusted p-value threshold.

A Cox regression for rs9582036 (SEQ ID NO. 2) and rs9554316 (SEQ ID NO.1) in the placebo arm failed to reveal a significant correlation with overall survival. Indeed, relative to rs9582036 (SEQ ID NO 2) AA carriers, the hazard ratio was 1.62 (CI=0.97-2.68; p=0.063) and 1.53 (CI=0.75-3.12; p=0.24) for rs9582036 (SEQ ID NO. 2) AC and CC carriers, respectively. Likewise, the hazard ratio for rs9554316 (SEQ ID NO: 1) TG and GG carriers was 1.34 (CI=0.81-2.22; p=0.25) and 1.36 (CI=0.60-3.04; p=0.46) relative to rs9554316 TT carriers. Consequently, a regression analysis assessing the interaction between genotypes and treatment was significant (p=0.023 for rs9582036 (SEQ ID NO. 2) and p=0.041 for rs9554316 (SEQ ID NO. 1)), indicating that rs9582036 (SEQ ID NO. 2) and rs9554316 (SEQ ID NO. 1) were predictive, rather than prognostic markers.

Analysis in the Sub-Group of Patients Treated with Bevacizumab, Adjusting for Ancillary Prognostic Factors: SNPs in VEGFR-1 are Independent Predictive Markers for Treatment

Overall Survival by Genotype - With covariates - Subset: Bev Arm

| | snp | gene | HR | CI_95 | pvalue | star | adj. pvalue | N/Nevts |
|---|---|---|---|---|---|---|---|---|
| 152 | rs9582036 | VEGFR1 | 1.921 | (1.27, 2.9) | 2.0e−03 | ** | 3.0e−01 | 71/63 |
| 150 | rs9554316 | VEGFR1 | 1.826 | (1.17, 2.86) | 8.9e−03 | ** | 1.0e+00 | 71/63 |
| 44 | rs1830792 | VEGFR1 | 1.723 | (1.14, 2.61) | 1.6e−02 | * | 1.0e+00 | 69/61 |
| 80 | rs3025035 | VEGF | 0.392 | (0.16, 0.95) | 1.7e−02 | * | 1.0e+00 | 68/60 |
| 123 | rs7324547 | VEGFR1 | 1.711 | (1.11, 2.64) | 2.1e−02 | * | 1.0e+00 | 71/63 |
| 136 | rs7995976 | VEGFR1 | 1.958 | (1.13, 3.4) | 2.2e−02 | * | 1.0e+00 | 69/61 |

To assess whether rs9582036 and rs9554316 correlated with overall survival independently from other co-variates known to affect treatment response, a Cox regression model was performed while adjusting for neutrophil counts, CRP levels and tumor location. Only patients with normal albumin levels were considered in this analysis.

Although the correlation with overall survival was attenuated, it was not suppressed and remained significant for both SNPs (p=0.002 and p=0.0089).

rs9582036 (SEQ ID NO. 2) has the lowest p-value and a p-value well below 0.05. rs9554316 (SEQ ID NO. 1) also has a p-value below 0.05. This further suggests that rs9582036 (SEQ ID NO. 2) and rs9554316 (SEQ ID NO. 1) are associated with overall survival in bevacizumab treated patients independently from other prognostic factors.

### Progression Free Survival

Analysis in the Sub-Group of Patients Treated with Bevacizumab

Progression Free Survival by Genotype - No covariates - Subset: Bev Arm

| | snp | gene | HR | CI_95 | pvalue | star | adj. pvalue | N/Nevts |
|---|---|---|---|---|---|---|---|---|
| 152 | rs9582036 | VEGFR1 | 1.889 | (1.32, 2.71) | 8.1e−04 | *** | 1.3e−01 | 77/72 |
| 150 | rs9554316 | VEGFR1 | 1.989 | (1.33, 2.98) | 1.2e−03 | ** | 1.9e−01 | 77/72 |
| 151 | rs9554320 | VEGFR1 | 1.442 | (1.05, 1.99) | 2.7e−02 | * | 1.0e+00 | 75/71 |
| 146 | rs9513070 | VEGFR1 | 1.503 | (1.05, 2.16) | 2.8e−02 | * | 1.0e+00 | 74/69 |
| 44 | rs1830792 | VEGFR1 | 1.537 | (1.05, 2.25) | 3.6e−02 | * | 1.0e+00 | 75/70 |
| 81 | rs3025039 | VEGF | 1.797 | (1.06, 3.05) | 3.9e−02 | * | 1.0e+00 | 77/72 |

We also analyzed whether rs9582036 (SEQ ID NO. 2) and rs9554316 (SEQ ID NO. 1) correlated with progression free survival. rs9582036 (SEQ ID NO.2) and rs9554316 (SEQ ID NO. 1) both have a very low p-value when looking at association with progression free survival in the bevacizumab treated group. This provides evidence that and are not only associated with overall survival but also risk of progression in patients treated with bevacizumab. In the bevacizumab arm, there was a clear and significant correlation with progression free survival. Relative to AA carriers, the hazard ratio for rs9582036 (SEQ ID NO. 2) AC and CC carriers was 1.86 (CI=1.11-3.10; p=0.018) and 3.63 (CI=1.64-8.05; p=0.0015). Such effect was not observed in the placebo arm. A similar situation was observed for the rs9554316 (SEQ ID NO. 1) SNP, showing a significant correlation with PFS in the bevacizumab arm, but not the placebo arm.

### VEGFR-1 SNPs Define a Locus in the VEGFR-1 Tyrosine-Kinase Domain

Since two SNPs in VEGFR-1 were significantly associated with bevacizumab-related outcome, and two other SNPs in VEGFR-1 also tended to be associated with outcome, the exact location of these SNPs in the VEGFR-1 gene as well as the haplotypes they represent as tagging SNPs, were studied in more detail. Intriguingly, all 4 SNPs were located close to each other (i.e., in introns 25, 27, 28 and 29) respectively for the rs9554320 (SEQ ID NO. 4), rs9582036 (SEQ ID NO: 2), rs9554316 (SEQ ID NO. 1) and rs9513070 (SEQ ID NO. 3) SNPs and represented 4 consecutive haplotypes in the VEGFR-1 gene. As a consequence of their proximate location, these 4 SNPs were also in significant linkage disequilibrium and did not occur independently from each other.

When considering the p-value of every SNP as a measure of its association with overall survival in the bevacizumab arm, and plotting these values in function of their location in the VEGFR-1 gene, a remarkable association signal encompassing exons 25 to 30 was noticed. Such association signal was not observed in the placebo group. Exon 25 to 30 of the VEGFR-1 gene code for amino acid residue 1029 to 1338, which are part of the receptor tyrosine-kinase (TK) domain in VEGFR-1 that contain two major auto-phosphorylation sites in the VEGFR-1 gene.

### EXAMPLE 2

In the context of the invention, genetic variability in underlying signalling pathways was explored in order to discover predictive patterns for anti angiogenic treatment. In this analysis, the correlation of genetic variability in the VEGF signaling pathway with clinical outcomes of patients with metastatic renal cell cancer (mRCC) treated with interferon alpha (IFN) plus bevacizumab or placebo in a phase III trial (AVOREN) was evaluated; see study number BO17705E; Escudier et al., Lancet 370: 2103-2111 (2007). Patients were randomized to receive bevacizumab (Avastin®) 10 mg/kg as an IV infusion every 2 weeks plus interferon alpha (IFN) starting at 9 MIU three times weekly (n=327) or placebo plus IFN (n=322). Patients were treated with bevacizumab (Avastin®) or placebo until disease progression or unacceptable toxicity. IFN was discontinued after a maximum of 52 weeks but bevacizumab (Avastin®) or placebo was continued until after disease progression or unacceptable toxicity.

### Samples and Single Nucleotide Polymorphism Selection

Germline DNA was available from 110 out of the 649 patients, of which 51 received interferon alpha plus placebo and 59 of which received interferon alpha plus bevacitumab; study number BO17705E; Escudier et al., Lancet 370: 2103-2111 (2007). As in Example 1, the sample from 1 patient of Asian ethnicity was not analysed. The four SNPs identified in Example 1 plus one additional VEGFA SNP (rs699947) were initially assessed.

### Genotyping

As in Example 1, peripheral blood was sampled in K2EDTA plastic Vacutainer tubes. After centrifugation, germ-line DNA was extracted from the precipitated leucocyte cell fraction according to standard procedures. Genotyping for the selected SNPs was carried out at the Vesalius Research Center using the Sequenom® iPLEX platform, as described previously by the manufacturer. All the SNPs that failed to provide robust genotypes in a first genotyping round were re-designed using a different set of PCR primers and tested again. In case of repetitive failure in the second design these SNPs were considered as failed. Of the 184 selected SNPs, 27 SNPs (14.7%) failed and 157 (85.3%) were successfully genotyped. The overall genotype success rate was 98.5%. At the individual sample level, all samples were genotyped with a high success rate (i.e., <5% of the samples had a success rate <95%, whereas >70% of the samples had a success rate >99%). No samples were excluded from the analysis and no significant deviations from Hardy-Weinberg were observed.

The amplification primers designed for SNPs rs9582036 (SEQ ID NO. 2), rs9554316 (SEQ ID NO. 1), rs9513070 (SEQ ID NO. 3) and rs9554320 (SEQ ID NO.4) are shown in FIG. 2. For rs9554316 (SEQ ID NO. 1), ACGTTGGATGATCGTAAAGACATCATTCG (SEQ ID NO. 25) and ACGTTGGATGTGCTGGAGACCATGACCAAG (SEQ ID NO. 26) were used. For SNP rs9582036 (SEQ ID NO. 2), ACGTTGGATGACTGTGCCCAGCAACAATAG (SEQ ID NO. 20) and ACGTTGGATGGCATAATAGCACTTTACTCC (SEQ ID NO. 21) were used. For rs9513070 (SEQ ID NO. 3), ACGTTGGATGGCAAGCTTGCCCAACTTGTG (SEQ ID NO. 35) and ACGTTGGATGGTTTGTGTTGGGCTGCACTC (SEQ ID NO. 36) were used. For rs9554320 (SEQ ID NO.4), ACGTTGGATGGTGGGAGGCCAGTTTGTAAC (SEQ ID NO. 30) and ACGTTGGATGAGCAAGGTTCCTGTGTGTAG (SEQ ID NO. 31) were used.

The unextended primers (probes) designed for SNPs rs9582036 (SEQ ID NO. 2), rs9554316 (SEQ ID NO. 1), rs9513070 (SEQ ID NO. 3) and rs9554320 (SEQ ID NO.4) are also shown in FIG. 2. For rs9554316 (SEQ ID NO. 1), AAGACATCATTCGATTTTTTTTCT (SEQ ID NO. 28) was used. For SNPs rs9582036 (SEQ ID NO. 2), AGCAACAATAGCCTTCTT (SEQ ID NO. 22) was used. For rs9513070 (SEQ ID NO. 3), CGTGTGGCCCACGGGCT (SEQ ID NO. 37) was used. For rs9554320 (SEQ ID NO.4), ACAGCGGCTTTGCAGTGC (SEQ ID NO. 32) was used.

### Statistics

When the allelic model was used, patients homozygous with the minor allele were coded as 2, heterozygous patients were coded as 1, and patients homozygous for the major allele were coded as 0. An independent, separate model was modelled for each SNP.

The Cox proportional hazard regression model was used to evaluate the effect of different genotypes Progression Free Survival.

In the Cox model, the time from entry in the study until disease progression or death was used as the Progression-Free Survival end-point.

For progression free survival, the null hypothesis stated that the genotype has no effect on risk of progression or survival. The likelihood ratio test was used to calculate a P value corresponding to this hypothesis for each tested SNP.

The primary analysis were conducted in the sub-group of patients in the bevacizumab arm of the trial. Additional analyses were performed in the sub-group of patients in the placebo arm of the trial, and treatment by genotype interaction was evaluated in model including both.

P-value below 0.05 were considered statistically significant. No correction for multiple testing was necessary in a confirmatory analysis of the findings from example 1.

Survival curves and median survival time were calculated using standard Kaplan-Meier estimates.

### Clinical Characteristics of Genetic Patient Population

All baseline demographics of the genetics subpopulation were similar to the overall study population.

| | | Placebo + IFN | | Bevacizumab + IFN |
|---|---|---|---|---|
| | | (Genotype | | (Genotype |
| | Placebo + IFN | population) | Bevacizumab + IFN | population) |
| | N = 322 | N = 51 | N = 327 | N = 59 |
| Sex | | | | |
| Female | 87 (27%) | 13 (25%) | 105 (32%) | 17 (29%) |
| Male | 235 (73%) | 38 (75%) | 222 (68%) | 42 (71%) |
| n | 322 | 51 | 327 | 59 |
| Age in years | | | | |
| Mean | 59.4 | 60.1 | 60.1 | 59.4 |
| SD | 10.89 | 10.74 | 10.12 | 10.32 |
| SEM | 0.61 | 1.50 | 0.56 | 1.34 |
| Median | 60.0 | 61.0 | 61.0 | 59.0 |
| Min-Max | 18-81 | 38-80 | 30-82 | 36-82 |
| n | 322 | 51 | 327 | 59 |
| Weight in kg | | | | |
| Mean | 77.52 | 76.86 | 75.99 | 76.36 |
| SD | 14.797 | 13.523 | 15.033 | 13.954 |
| SEM | 0.828 | 1.894 | 0.831 | 1.817 |
| Median | 76.00 | 76.90 | 75.00 | 78.00 |
| Min-Max | 42.0-121.0 | 46.5-118.0 | 40.0-115.0 | 54.0-109.0 |
| n | 319 | 51 | 327 | 59 |
| Height in cm | | | | |
| Mean | 169.9 | 170.2 | 169.8 | 171.5 |
| SD | 8.84 | 8.82 | 9.07 | 9.24 |
| SEM | 0.50 | 1.25 | 0.50 | 1.20 |
| Median | 170.5 | 171.0 | 170.0 | 173.0 |
| Min-Max | 147-198 | 152-191 | 146-194 | 178-190 |
| n | 314 | 50 | 325 | 59 |
| Body Surface | | | | |
| Area (m²) | | | | |
| Mean | 1.89 | 1.88 | 1.87 | 1.92 |
| SD | 0.199 | 0.196 | 0.207 | 0.198 |
| SEM | 0.011 | 0.028 | 0.011 | 0.026 |
| Median | 1.87 | 1.88 | 1.86 | 1.91 |
| Min-Max | 1.4-2.5 | 1.4-2.5 | 1.3-2.4 | 1.5-2.3 |
| n | 312 | 50 | 325 | 59 |
| Age Category | | | | |
| (years) | | | | |
| <65 | 204 (63%) | 30 (59%) | 206 (63%) | 37 (63%) |
| >=65 | 118 (37%) | 21 (41%) | 121 (37%) | 22 (37%) |
| n | 322 | 51 | 327 | 59 |
| Race | | | | |
| Black | 1 (<1%) | — | 2 (<1%) | — |
| Other | 1 (<1%) | — | 2 (<1%) | — |
| Caucasian/White | 312 (97%) | 51 (100%) | 312 (95%) | 58 (98%) |
| Asian | 8 (2%) | — | 11 (3%) | 1 (2%) |
| n | 322 | 51 | 327 | 59 |
| Smoking Status | | | | |
| at Screening | | | | |
| Never smoked | 148 (46%) | 22 (43%) | 154 (47%) | 30 (51%) |
| Past smoker | 129 (40%) | 20 (39%) | 126 (39%) | 23 (39%) |
| Current smoker | 43 (13%) | 9 (18%) | 45 (14%) | 6 (10%) |
| n | 320 | 51 | 325 | 59 |

### Summary of Progression Free Survival

The progression-free survival Kaplan Meier curves cross at the same time the overall population and the genotype population (FIGS. 11 and 12). But the genotype population had overall a better clinical outcome than the overall population (see table below, time to event).

| | | | Placebo + IFN | Bevacizumab + IFN |
|---|---|---|---|---|
| | | | (Genotype | (Genotype |
| | Placebo + IFN | Bevacizumab + IFN | population) | population) |
| | N = 322 | N = 327 | N = 51 | N = 59 |
| Patients with events | 298 (92.5%) | 301 (92.0%) | 42 (82.4%) | 56 (94.9%) |
| Patients without events | 24 (7.5%) | 26 (8.0%) | 9 (17.6%) | 3 (5.1%) |
| Time to event | | | | |
| (months) | | | | |
| Median # | 5.5 | 10.2 | 8.7 | 15.5 |
| 95% CI for | [4.2; 5.7] | [7.7; 11.1] | [7.2; 14.2] | [13.5; 18.4] |
| Median # | | | | |
| 25% and 75%-ile | 2.0; 10.4 | 3.7; 16.6 | 4.0; 20.1 | 9.4; 21.7 |
| Range | 0.5 to 45.8 | 0.4 to 44.1 | 1.8 to 41.1 | 1.4 to 41.3 |

| | | |
|---|---|---|
| p-Value (Log- | 0.0004 | 0.7345 |
| Rank Test) | | |
| p-Value | <0.0001 | 0.0561 |
| (Wilcoxon Test) | | |
| Hazard Ratio | 0.75 | 0.93 |
| 95% CI | [0.64; 0.88] | [0.62; 1.40] |

SNPs in VEGFR-1 correlated with progression-free survival in the bevacizumab treatment arm.

Analysis in Placebo Treated Group (Progression-Free Survival, without Covariates, S_BEV=0)

| | snp | gene | HR | CI_95 | pvalue | star | holm_pval | N/Nevts | geno. freq |
|---|---|---|---|---|---|---|---|---|---|
| 1 | rs699947 | VEGF | 0.632 | (0.4, 1) | 4.70e−02 | * | 2.35e−01 | 45/37 | 14/22/9 |
| 5 | rs9582036 | VEGFR1 | 0.768 | (0.5, 1.19) | 2.25e−01 | | 8.99e−01 | 45/37 | 21/17/7 |
| 3 | rs9554316 | VEGFR1 | 0.826 | (0.47, 1.44) | 4.93e−01 | | 1.00e+00 | 45/37 | 26/17/2 |
| 2 | rs9513070 | VEGFR1 | 1.109 | (0.59, 2.08) | 7.48e−01 | | 1.00e+00 | 45/37 | 11/26/8 |
| 4 | rs9554320 | VEGFR1 | 0.977 | (0.64, 1.49) | 9.14e−01 | | 1.00e+00 | 45/37 | 14/21/10 |

No genotypes were detected to be associated with progression-free survival in the patients treated with placebo.

Analysis in Bevacizumab Treated Group (Progression-Free Survival, without Covariates, S_BEV=1)

| | snp | gene | HR | CI_95 | pvalue | star | holm_pval | N/Nevts | geno. freq |
|---|---|---|---|---|---|---|---|---|---|
| 2 | rs9513070 | VEGFR1 | 1.679 | (1.12, 2.51) | 1.18e−02 | * | 5.92e−02 | 56/54 | 19/28/9 |
| 3 | rs9554316 | VEGFR1 | 1.812 | (1.08, 3.05) | 3.26e−02 | * | 1.30e−01 | 55/53 | 36/18/1 |
| 5 | rs9582036 | VEGFR1 | 1.262 | (0.85, 1.88) | 2.63e−01 | | 7.90e−01 | 56/54 | 30/20/6 |
| 1 | rs699947 | VEGF | 1.178 | (0.81, 1.71) | 3.92e−01 | | 7.90e−01 | 55/53 | 19/24/12 |
| 4 | rs9554320 | VEGFR1 | 1.115 | (0.76, 1.63) | 5.76e−01 | | 7.90e−01 | 52/50 | 20/22/10 |

Two SNPs, rs9513070 (SEQ ID NO: 3) and rs9554316 (SEQ ID NO:1), in the VEGFR-1 gene correlated with progression-free survival in the bevacizumab-treated group only (see FIGS. 13 to 16). AA carriers of rs9513070 (SEQ ID NO: 3) showed increased progression free survival in comparison to AG and GG carriers in the bevacizumab-treated group (FIG. 13). GG carriers of rs9554316 (SEQ ID NO:1) showed increased progression free survival in comparison to GT and TT carriers in the bevacizumab-treated group only (FIG. 15).

1-2. (canceled)

3. A method for improving overall survival of a patient suffering from a malignant disease comprising administering to the patient an effective amount of bevacizumab, wherein the patient is pre-determined to have one or more variant alleles of the VEGFR-1 gene.

4. A method for improving progression-free survival of a patient suffering from a malignant disease comprising administering to the patient an effective amount of bevacizumab, wherein the patient is pre-determined to have one or more variant alleles of the VEGFR-1 gene.

5. An in vitro method for the identification of a responder for or a patient sensitive to bevacizumab, said method comprising determining from a patient sample if a patient suffering from a malignant disease has one or more variant alleles of the VEGFR-1 gene, whereby the presence of the one or more variant alleles of the VEGFR-1 gene is indicative for a responding patient or is indicative for a sensitivity of the patient to bevacizumab.

6. A method for providing an improved chemotherapy regimen for a patient suffering from a malignant disease comprising administering to the patient an effective amount of bevacizumab, wherein the patient is pre-determined to have one or more variant alleles of the VEGFR-1 gene.

7. The method according to claim 3, wherein the patient sample is a blood sample.

8-9. (canceled)

10. The method according to claim 3, further comprising co-treating the patient by administering to the patient an effective amount of a chemotherapeutic agent.

11. The method according to claim 10, wherein the chemotherapeutic agent is selected from the group consisting of interferon alpha, 5-fluorouracil, leucovorin, irinotecan, gemcitabine-erlotinib and platinum-based chemotherapeutic agents.

12-13. (canceled)

14. The method according to claim 3, wherein the malignant disease is pancreatic cancer.

15. The method according to claim 14, wherein further comprising co-treating the patient by administering to the patient an effective amount of gemcitabine-erlotinib.

16. The method according to claim 3, wherein the malignant disease is renal cell cancer.

17. The method according to claim 16, wherein further comprising co-treating the patient by administering to the patient an effective amount of interferon alpha.

18. The method according to claim 3, wherein the one or more variant alleles of the VEGFR-1 gene are in the region encompassing the tyrosine-kinase domain of the VEGFR-1 gene.

19. The method according to claim 3, wherein the one or more variant alleles of the VEGFR-1 gene are in the region encompassing exons 25 to 30 of the VEGFR-1 gene.

20. The method according to claim 3, wherein the one or more variant alleles of the VEGFR-1 gene are in an intron region of the VEGFR-1 gene.

21-22. (canceled)

23. The method according to claim 14, wherein the more variant allele of the VEGFR-1 gene is selected from the group consisting of:
(a) rs9554316 (SEQ ID NO:1);
(b) a sequence corresponding to rs9554316 (SEQ ID NO. 1) or having at least 80% homology to rs9554316 (SEQ ID NO. 1) having a substitution, deletion or addition of at least one nucleotide corresponding to position 51 of rs9554316 (SEQ ID NO. 1);
(c) a sequence corresponding to rs9554316 (SEQ ID NO. 1) or having at least 80% homology to rs9554316 (SEQ ID NO. 1), wherein position 51 is a T or G and having a substitution, deletion or addition of at least one nucleotide at any position from position 1 to 50 and 52 to 101; and
(d) rs9554316 (SEQ ID NO. 1), wherein position 51 is a G.

24. The method according to claim 16, wherein the variant allele of the VEGFR-1 gene is selected from the group consisting of:
(a) rs9554316 (SEQ ID NO. 1);
(b) a sequence corresponding to rs9554316 (SEQ ID NO. 1) or having at least 80% homology to rs9554316 (SEQ ID NO. 1) having a substitution, deletion or addition of at least one nucleotide corresponding to position 51 of rs9554316 (SEQ ID NO. 1);
(c) a sequence corresponding to rs9554316 (SEQ ID NO. 1) or having at least 80% homology to rs9554316 (SEQ ID NO. 1), wherein position 51 is a T or G and having a substitution, deletion or addition of at least one nucleotide at any position from position 1 to 50 and 52 to 101; and
(d) rs9554316 (SEQ ID NO. 1), wherein position 51 is a G.

25. The method according to claim 23, further comprising determining if the patient has more variant alleles of the VEGFR-1 gene selected from the group consisting of:
rs9582036 (SEQ ID NO. 2), rs9513070 (SEQ ID NO. 3), and rs9554320 (SEQ ID NO. 4).

26. The method according to claim 24, further comprising determining if the patient has the variant alleles of the VEGFR-1 gene of
rs9513070 (SEQ ID NO. 3)

27-30. (canceled)

31. The method according to claim 23, further comprising determining if the patient has one or more variant alleles of the VEGFR-1 gene selected from the group consisting of rs17619037 (SEQ ID NO. 5), rs9579177 (SEQ ID NO. 6), rs9579176 (SEQ ID NO. 7), rs9554319 (SEQ ID NO. 8), rs7996030 (SEQ ID NO. 9), rs9513075 (SEQ ID NO. 10), rs7993418 (SEQ ID NO. 11), rs9513071 (SEQ ID NO. 12), rs12429309 (SEQ ID NO. 15), rs7982251 (SEQ ID NO 16), rs9508016 (SEQ ID NO. 17), rs7982957 (SEQ ID NO. 18), rs3794400 (SEQ ID NO. 19), rs3794395 (SEQ ID NO. 52), rs9554317 (SEQ ID NO. 53), rs9513073 (SEQ ID NO. 54), rs9513074 (SEQ ID NO. 55), rs9508015 (SEQ ID NO. 56), rs2011950 (SEQ ID NO. 57), rs9513110 (SEQ ID NO. 58), rs9513112 (SEQ ID NO. 59), rs9513113 (SEQ ID NO. 60), rs9551471 (SEQ ID NO. 61), rs2296285 (SEQ ID NO. 62), rs9513116 (SEQ ID NO. 63), rs9551473 (SEQ ID NO. 64), rs7330109 (SEQ ID NO. 65), rs9508037 (SEQ ID NO. 66), rs1924981 (SEQ ID NO. 67), rs34140996 (SEQ ID NO. 68), rs7985584 (SEQ ID NO. 69), rs7992940 (SEQ ID NO. 70) and rs718273 (SEQ ID NO. 71).

32. The method according to claim 31, further comprising determining if the patient has one or more variant alleles selected from the group consisting of rs45455097 (SEQ ID NO. 40), rs1886233 (SEQ ID NO. 41), rs9554309 (SEQ ID NO. 42), rs11619230 (SEQ ID NO. 43), rs9554311 (SEQ ID NO. 44), rs11620238 (SEQ ID NO. 13), rs17618631 (SEQ ID NO. 14), rs4771233 (SEQ ID NO. 45), rs6491274 (SEQ ID NO. 46), rs7982639 (SEQ ID NO. 47), rs12877718 (SEQ ID NO. 48), rs10507382 (SEQ ID NO. 49), rs57354941 (SEQ ID NO. 50) and rs17086497 (SEQ ID NO. 51).

33. (canceled)

34. A kit for predicting the response to or sensitivity to bevacizumab therapy of a patient suffering or suspected to suffer from metastatic pancreatic cancer or renal cell cancer comprising primers and/or probes capable of detecting the variant allele of rs9554316 (SEQ ID NO. 1), wherein position 51 of rs9554316 (SEQ ID NO. 1) is a G.

35-38. (canceled)

39. The kit according to claim 34, wherein
(a) the primers are selected from the group consisting of SEQ ID NO. 25 and SEQ ID NO. 26; or
(b) the probes are selected from the group consisting of SEQ ID NO: 27, SEQ ID NO:28, and SEQ ID NO:29.

40-41. (canceled)

42. A method for treating a patient suffering from pancreatic cancer comprising administering an effective amount of bevacizumab to the patient, wherein the patient is pre-determined to have one or more variant alleles of the VEGFR-1 gene.

43. The method according to claim 42, wherein the one or more variant alleles of the VEGFR-1 gene is selected from the group consisting of rs9554316 (SEQ ID NO. 1), rs9582036 (SEQ ID NO. 2), rs9513070 (SEQ ID NO. 3), and rs9554320 (SEQ ID NO. 4).

44. The method according to claim 42, wherein the one or more variant alleles of the VEGFR-1 gene is selected from the group consisting of rs9554316 (SEQ ID NO. 1) and rs9582036 (SEQ ID NO. 2).

45. The method according to claim 42, wherein the variant alleles of the VEGFR-1 gene is rs9554316 (SEQ ID NO. 1), and wherein position 51 of rs9554316 (SEQ ID NO. 1) is a G.

46. The method according to claim 42, wherein the variant allele of the VEGFR-1 gene is rs9582036 (SEQ ID NO. 2), and wherein position 51 of rs9582036 (SEQ ID NO. 2) is an A.

47. The method according to claim 42, further comprising co-treating the patient by administering to the patient an effective amount of gemcitabine-erlotinib.

48. A method for treating a patient suffering from renal cell cancer comprising administering an effective amount of bevacizumab to the patient, wherein the patient is pre-determined to have one or more variant alleles of the VEGFR-1 gene.

49. The method according to claim 48, wherein the one or more variant alleles of the VEGFR-1 gene is selected from the group consisting of rs9554316 (SEQ ID NO. 1) and rs9513070 (SEQ ID NO. 3).

50. The method according to claim 48, wherein the variant allele of the VEGFR-1 gene is rs9554316 (SEQ ID NO. 1), and wherein position 51 of rs9554316 (SEQ ID NO. 1) is a G.

51. The method according to claim 48, wherein the variant allele of the VEGFR-1 gene is rs9513070 (SEQ ID NO. 3), and wherein position 51 of rs9513070 (SEQ ID NO. 3) is an A.

52. The method according to claim 48, further comprising co-treating the patient by administering to the patient an effective amount of interferon alpha.

53. A method for identifying a patient suffering from a malignant disease who is likely to be sensitive to bevacizumab treatment comprising contacting a sample from the patient with a complementary oligonucleotide and determining whether the patient sample contains one or more variant alleles of the VEGFR-1 gene selected from the group consisting of rs9554316 (SEQ ID NO. 1), rs9582036 (SEQ ID NO. 2), rs9513070 (SEQ ID NO. 3), and rs9554320 (SEQ ID NO. 4), wherein the presence of one or more variant alleles of the VEGFR-1 gene in the patient sample indicates that the patient is likely to be sensitive to bevacizumab treatment.

54. The method according to claim 53, wherein the VEGFR-1 gene allele variant is rs9554316 (SEQ ID NO. 1), and position 51 of rs9554316 (SEQ ID NO. 1) is a G.

55. The method according to claim 53, wherein the VEGFR-1 gene allele variant rs9582036 (SEQ ID NO. 2), and position 51 of rs9582036 (SEQ ID NO. 2) is an A.

56. The method according to claim 53, wherein the VEGFR-1 gene allele variant is rs9513070 (SEQ ID NO. 3), and position 51 of rs9513070 (SEQ ID NO. 3) is an A.

57. The method according to claim 53, wherein the VEGFR-1 gene allele variant is rs9554320 (SEQ ID NO. 4), and position 51 of rs9554320 (SEQ ID NO. 4) is a C.

58. The method according to claim 53, wherein the malignant disease is pancreatic cancer.

59. The method according to claim 58, wherein the one or more variant alleles of the VEGFR-1 gene are selected from the group consisting of rs9554316 (SEQ ID NO. 1) and rs9582036 (SEQ ID NO. 2).

60. The method according to claim 58, wherein the patient is being co-treated with gemcitabine-erlotinib.

61. The method according to claim 53, wherein the malignant disease is renal cell cancer, and wherein the one or more variant alleles of the VEGFR-1 gene are selected from the group consisting of rs9554316 (SEQ ID NO. 1) and rs9513070 (SEQ ID NO. 3).

62. The method according to claim 61, wherein the patient is being co-treated with interferon alpha.

63. The method according to claim 53, wherein the complementary oligonucleotide is a probe selected from the group consisting of SEQ ID NO: 27, SEQ ID NO:28, and SEQ ID NO:29.


SEQUENCE LISTING (71 sequences)

SEQ ID NO: 1 - DNA, 101 bp, Homo sapiens
atagacacaaattctaagaaatcgtaaagacatcattcgattttttttctkgaactcaatggaattctcagcccccagcccttggtcatggtctccagcaa

SEQ ID NO: 2 - DNA, 101 bp, Homo sapiens
tgctgcgattacaggcacgagccactgtgcccagcaacaatagccttcttmcaaaatgtatactaaagtatttaggagtaaagtgctattatgccttaagc

SEQ ID NO: 3 - DNA, 101 bp, Homo sapiens
ttgctttttatactataatgcaagcttgcccaacttgtggcccacgggctrcatgaatcccaggatggctttgagtgcagcccaacacaaactcgtaaact

SEQ ID NO: 4 - DNA, 101 bp, Homo sapiens
tttccggggctgtgagcaaggttcctgtgtgtagctgatcattctcccccmgcactgcaaagccgctgttacaaactggcctcccaccactcaaggcacac

SEQ ID NO: 5 - DNA, 101 bp, Homo sapiens
agtgcaaaacatccacaaagccccaggaaaaaaagtgtccaagacaggcargcaagtgcaatttgcacagtggagaaagcctcacatggacacccgaaaag

SEQ ID NO: 6 - DNA, 101 bp, Homo sapiens
acattactggcgttggtgtttggaagggatctgaagaaggggtctatcggrgtgcactaggtaccttaacttcacgacttacatcaaacatggaggtggca

SEQ ID NO: 7 - DNA, 101 bp, Homo sapiens
ccccttagcccctcaaccccacatcacagggctttagagagaagagctggragacctcttgtttgccaggctatactggttagcttctcccattgtgattg

SEQ ID NO: 8 - DNA, 101 bp, Homo sapiens
cttcttccaaaatgtatactaaagtatttaggagtaaagtgctattatgcyttaagcctccttttaaatggtttggcaaaaagaaaaatccgtaacaaaca

SEQ ID NO: 9 - DNA, 101 bp, Homo sapiens
ctcccagagggacctactctggtccgttgtgaaatcagagaatgccagacrtaggagagactggaggcatcatctcgtctgagtgcccaatggcacaggag

SEQ ID NO: 10 - DNA, 101 bp, Homo sapiens
gaggacaccacgcaccagtgacaagaacaggtcccctcccgcttccctacrctcaggcagagctccccagccctgctgttgggcctgagtctcctgtccac

SEQ ID NO: 11 - DNA, 101 bp, Homo sapiens
tcttcaaaggttttgattctttccaggctcatgaacttgaaagcatttacrtatctaatgaagaaacagaaagaattatcaagacaggaaaaggcatccag

SEQ ID NO: 12 - DNA, 101 bp, Homo sapiens
ttacttgtgctgggagatggcggttgctggggaaaaaacctgagaaaccckagttcatatcctccctctgacacttcttagaggtatgcctttagggggcg

SEQ ID NO: 13 - DNA, 101 bp, Homo sapiens
aggaagtgcatgatcttttctgacctggcctccaagccatgcagtgtcccktctttcacattgcattgatcacaagtgagtcctaaagctagctctgattc

SEQ ID NO: 14 - DNA, 101 bp, Homo sapiens
cttcaagtcatttgcctgaatgaatgattttattcttatgttatgaaaccsataatactttttaaaggatttcagattcttgaagacaaaatgcctcttgc

SEQ ID NO: 15 - DNA, 101 bp, Homo sapiens
gcagtgtatgagggttccaatttctccatatcctcgccacactgacttcayctagctatcctcatgggcgtaaagtggtatcttatggtaatttctttccc

SEQ ID NO: 16 - DNA, 101 bp, Homo sapiens
gtggctgtctgcagggacatgctctgcattcagagagaacactggggagaygacacgtttctctttttgtgaaacatgctcccgtagtgattactaaatga

SEQ ID NO: 17 - DNA, 101 bp, Homo sapiens
agggaggacatgggaggtcatccacaaaagtgaaaaataacactatgtctraaaacaagcagggaagagaagccagtacgagccagcggggagatgctagg

SEQ ID NO: 18 - DNA, 101 bp, Homo sapiens
agaacactggggagacgacacgtttctctttttgtgaaacatgctcccgtwgtgattactaaatgacattcctcccagagcatgcaacacgggcagagcta

SEQ ID NO: 19 - DNA, 101 bp, Homo sapiens
agaaggacagaaatacgtttgaggaggggagaggcaaaagccagcctcccygcctcttaaccagacccttgcgagtagagcggcttctggttactgtcaca

SEQ ID NO: 20 - DNA, 30 bp, Artificial Sequence
  source: /note=“description of artificial sequence an artificially synthesized primer for PCR”
acgttggatgactgtgcccagcaacaatag

SEQ ID NO: 21 - DNA, 30 bp, Artificial Sequence
  source: /note=“description of artificial sequence an artificially synthesized primer for PCR”
acgttggatggcataatagcactttactcc

SEQ ID NO: 22 - DNA, 18 bp, Artificial Sequence
  source: /note=“description of artificial sequence an artificially synthesized probe”
agcaacaatagccttctt

SEQ ID NO: 23 - DNA, 19 bp, Artificial Sequence
  source: /note=“description of artificial sequence an artificially synthesized probe or PCR extension product”
agcaacaatagccttcttc

SEQ ID NO: 24 - DNA, 19 bp, Artificial Sequence
  source: /note=“description of artificial sequence an artificially synthesized probe or PCR extension product”
agcaacaatagccttctta

SEQ ID NO: 25 - DNA, 29 bp, Artificial Sequence
  source: /note=“description of artificial sequence an artificially synthesized primer for PCR”
acgttggatgatcgtaaagacatcattcg

SEQ ID NO: 26 - DNA, 30 bp, Artificial Sequence
  source: /note=“description of artificial sequence an artificially synthesized primer for PCR”
acgttggatgtgctggagaccatgaccaag

SEQ ID NO: 27 - DNA, 24 bp, Artificial Sequence
  source: /note=“description of artificial sequence an artificially synthesized probe”
aagacatcattcgattttttttct

SEQ ID NO: 28 - DNA, 25 bp, Artificial Sequence
  source: /note=“description of artificial sequence an artificially synthesized probe or PCR extension product”
aagacatcattcgattttttttctg

SEQ ID NO: 29 - DNA, 25 bp, Artificial Sequence
  source: /note=“description of artificial sequence an artificially synthesized probe or PCR extension product”
aagacatcattcgattttttttctt

SEQ ID NO: 30 - DNA, 30 bp, Artificial Sequence
  source: /note=“description of artificial sequence an artificially synthesized primer for PCR”
acgttggatggtgggaggccagtttgtaac

SEQ ID NO: 31 - DNA, 30 bp, Artificial Sequence
  source: /note=“description of artificial sequence an artificially synthesized primer for PCR”
acgttggatgagcaaggttcctgtgtgtag

SEQ ID NO: 32 - DNA, 18 bp, Artificial Sequence
  source: /note=“description of artificial sequence an artificially synthesized probe”
acagcggctttgcagtgc

SEQ ID NO: 33 - DNA, 19 bp, Artificial Sequence
  source: /note=“description of artificial sequence an artificially synthesized probe or PCR extension product”
acagcggctttgcagtgcg

SEQ ID NO: 34 - DNA, 19 bp, Artificial Sequence
  source: /note=“description of artificial sequence an artificially synthesized probe or PCR extension product”
acagcggctttgcagtgct

SEQ ID NO: 35 - DNA, 30 bp, Artificial Sequence
  source: /note=“description of artificial sequence an artificially synthesized primer for PCR”
acgttggatggcaagcttgcccaacttgtg

SEQ ID NO: 36 - DNA, 30 bp, Artificial Sequence
  source: /note=“description of artificial sequence an artificially synthesized primer for PCR”
acgttggatggtttgtgttgggctgcactc

SEQ ID NO: 37 - DNA, 17 bp, Artificial Sequence
  source: /note=“description of artificial sequence an artificially synthesized probe”
cgtgtggcccacgggct

SEQ ID NO: 38 - DNA, 18 bp, Artificial Sequence
  source: /note=“description of artificial sequence an artificially synthesized probe or PCR extension product”
cgtgtggcccacgggcta

SEQ ID NO: 39 - DNA, 18 bp, Artificial Sequence
  source: /note=“description of artificial sequence an artificially synthesized probe or PCR extension product”
cgtgtggcccacgggctg

SEQ ID NO: 40 - DNA, 101 bp, Homo sapiens
tgtgtttttttaaaattttttaaaaaccaatttcttaatactttacatttyattattaatatttatacttctaagtatttttcctaaggaaaaagtacttc

SEQ ID NO: 41 - DNA, 101 bp, Homo sapiens
tactgggagaaaaattatggagcatcaactgggtaaactataatacaaacraacgttaagcgtgaagggatagaaacatagggcagtgtttgtgtaaaaat

SEQ ID NO: 42 - DNA, 101 bp, Homo sapiens
taaataatttcatagaagttcagtatagtacgtaattcttgtaagagtatygtatgcaagctctctgtatgccacccactgttagttcaaattgagatttt

SEQ ID NO: 43 - DNA, 101 bp, Homo sapiens
tgtatgccacccactgttagttcaaattgagatttttgttttgttttgttyttaagaacataccaagaaaataccagtgaattgttggatatgaattccct

SEQ ID NO: 44 - DNA, 101 bp, Homo sapiens
aaattgagatttttgttttgttttgttcttaagaacataccaagaaaataycagtgaattgttggatatgaattccctgttagttgatttaaatcctttct

SEQ ID NO: 45 - DNA, 101 bp, Homo sapiens
gagcccagagttttgagacaggcctgggcaacatagtgagaccttgtctcyacaaaaagtaaaatcagccaggcatggtgtcacgcacctgtagccccagc

SEQ ID NO: 46 - DNA, 101 bp, Homo sapiens
ggagcaccttcacatggcctccatgtggcttaggtttctccaagtatggcygctgggtccctggcgggaactgttgaagagtgagtgtaccaagactcagg

SEQ ID NO: 47 - DNA, 101 bp, Homo sapiens
ttcaaaagtcagggaaaagaggtaaaaccagcaaaagagacaggtagcctrtgagatgggaagaagaaagtatcatgtcctggaaccaaactgaagttgtt

SEQ ID NO: 48 - DNA, 101 bp, Homo sapiens
ataagtgtgttcctttaaagcactttaaatagttgctggcacatattgggyactcaaatgatagcaatgatagcaattactagctatcactgcttcccaaa

SEQ ID NO: 49 - DNA, 101 bp, Homo sapiens
tgggaaaaagaaggataatataggtatgtttcaaaacaaatatattttagyctgagattaagcccttccaccttttaaaatgggttttctcctatgaaaag

SEQ ID NO: 50 - DNA, 101 bp, Homo sapiens
aacattagtagcatctggttggattttgggttttttttttccccctccctyaagtgttcttagctcgcagaatagaagaatggccatccctgtgttgggat

SEQ ID NO: 51 - DNA, 101 bp, Homo sapiens
agtttccagagccatgagaacatttctaaactgacgtgacattgattcctmaagcattcttcaagtcatttgcctgaatgaatgattttattcttatgtta

SEQ ID NO: 52 - DNA, 101 bp, Homo sapiens
tggcatggctacacggctaaatattatttaatggcataatcgagtgctagygccaggcttgtattttgtgtgtgtgtgtgattacagattgggagaattgg

SEQ ID NO: 53 - DNA, 101 bp, Homo sapiens
ccatctcggcctcccagagtgctgggattacaggcgtgagccactgtgccyggccgcaagcagtattttctgactgctgcagcttcttcccatatcttccc

SEQ ID NO: 54 - DNA, 101 bp, Homo sapiens
tatgtaaagacttgaaggtttgagaaagtccaaagcagtttttggtccaargctgatgtgactttgtacctctgagttatttgcgaccacagcctgggtat

SEQ ID NO: 55 - DNA, 101 bp, Homo sapiens
atgcttaaaatagtggcacttataagcaccaaataactatttaatcagtaytattcaatactgttataataaaaactaatttcaggccaggcatggaggct

SEQ ID NO: 56 - DNA, 101 bp, Homo sapiens
tagtggcacttataagcaccaaataactatttaatcagtactattcaataytgttataataaaaactaatttcaggccaggcatggaggctcacgcctgta

SEQ ID NO: 57 - DNA, 101 bp, Homo sapiens
ctgcaatctccgcttcataggctcaagtgattctcatgcctcagcctcccragtagctgccaccaggcccggctaacttttgtatttttagtagagacggg

SEQ ID NO: 58 - DNA, 101 bp, Homo sapiens
aacagatctttgttttagaaagaagaaaagacaatccagaaacaaccaggkggactctagacacccatggaggaggtgatcagagctcatgagagtaccag

SEQ ID NO: 59 - DNA, 101 bp, Homo sapiens
tagctctatagtaggcagaaatatcctttaagttcattaaaggttctcagrtcttatttaaagaaatgtaccccaatgctagcaatgattacaaccttgca

SEQ ID NO: 60 - DNA, 101 bp, Homo sapiens
cgttacacgtttttgttgctaagttattccatttccatggcgagaaatgtygatgaaaaacaacactagcttgaggctgatgcattaatctttcatacata

SEQ ID NO: 61 - DNA, 101 bp, Homo sapiens
acaatcccttttcttccacctagaatcatgcaatagaaataccagtctacrctcttcaaaacaaaacccccagacatcaaaagacagtatctatgcatatc

SEQ ID NO: 62 - DNA, 101 bp, Homo sapiens
ccagttgaaaaagggggaaaaaaatcaaattaccttcccccaacagattawaaaggaaataccaaacagccacaaagaagctcgaaaccttttctatcgcc

SEQ ID NO: 63 - DNA, 101 bp, Homo sapiens
ctctaaaagccaataccaatttcagcactgggtccactgaaattctatccraagatatcagaacttcagtgatttgtgtccctccaaacactatgcaaaac

SEQ ID NO: 64 - DNA, 101 bp, Homo sapiens
acatataaaaagaattatatacaatgcccaagtgggatttttttcaggaakgcaagattggtttaacatccaaaaaccaatgtaattactatatcatgtca

SEQ ID NO: 65 - DNA, 101 bp, Homo sapiens
taaaatggcagaagaaggatttgacaaaaatccaagctcctttcgtgatgwaaacactcaggaaaatgggaattgcactgaatttcctcaacctaataaat

SEQ ID NO: 66 - DNA, 101 bp, Homo sapiens
agaaattaaagaagatctactaagtgggaaacatctcatgttcatggattrgaagacttaatattgttaagatgttaatgtcccccaaattgatctacaga

SEQ ID NO: 67 - DNA, 101 bp, Homo sapiens
atacatgaaagaacctcaaaaatattatgctgagtgaaagaaattagtcayaaaagactacaaattatgtattctatttatataaaatatccatgagacaa

SEQ ID NO: 68 - DNA, 101 bp, Homo sapiens
ataaggggtgatagctaaaggctatggagtttattttgagacaatgacatrttctaaaattgaccgtgatgatgatggttgcatatatctgtcaatatgct

SEQ ID NO: 69 - DNA, 101 bp, Homo sapiens
tgtcaatatgctacaaaccactgaatcatcattttaaatgggtgtggtatrtaaattatatctcaataaaacttattttgaattaaaacagaggtttagtc

SEQ ID NO: 70 - DNA, 101 bp, Homo sapiens
aataagaagtgcctctttctaaaaaagagctctttgcctactacctgcctktcctcagaagaaagacattgctttttgttttgttttgttttgtttgtttt

SEQ ID NO: 71 - DNA, 101 bp, Homo sapiens
ccttgaagctattactgaccaatcagaatactatcactaaggatctctctygcctccttcctcccctcatccctcagtctctcttctccttctccctcttc